(19) United States
(12) Patent Application Publication
(10) Pub. No.: US 2025/0037794 A1
(43) Pub. Date: Jan. 30, 2025

(54) GROUP OF SNP MARKERS RELATED TO MYOPIA OR HIGH MYOPIA AND USE THEREOF

(71) Applicants: EYE HOSPITAL, WENZHOU MEDICAL UNIVERSITY, Wenzhou (CN); CENTER OF OPTOMETRY INTERNATIONAL INNOVATION OF WENZHOU, Wenzhou (CN)

(72) Inventors: Liangde Xu, Wenzhou (CN); Hong Wang, Wenzhou (CN); Jia Qu, Wenzhou (CN)

(73) Assignees: EYE HOSPITAL, WENZHOU MEDICAL UNIVERSITY, Wenzhou (CN); CENTER OF OPTOMETRY INTERNATIONAL INNOVATION OF WENZHOU, Wenzhou (CN)

(21) Appl. No.: 18/914,251

(22) Filed: Oct. 13, 2024

Related U.S. Application Data
(63) Continuation of application No. PCT/CN2022/100395, filed on Jun. 22, 2022.

(30) Foreign Application Priority Data
Apr. 13, 2022 (CN) CN202210386945.6
Apr. 13, 2022 (CN) CN202210386953.0
Apr. 13, 2022 (CN) CN2022103869668
Apr. 13, 2022 (CN) CN2022103869812
Apr. 13, 2022 (CN) CN2022103869846
Apr. 13, 2022 (CN) CN2022103884386
Apr. 13, 2022 (CN) CN2022103884649
Apr. 13, 2022 (CN) CN2022103884901
Apr. 13, 2022 (CN) CN2022103885016
May 13, 2022 (CN) CN2022105247490

(51) Int. Cl. G16B 20/20; G16B 40/20; G16H 50/20; G16H 50/30

(52) U.S. Cl. CPC G16B 20/20; G16B 40/20; G16H 50/20; G16H 50/30

(57) ABSTRACT

The present invention belongs to the technical fields of biomedicine and gene detection, and particularly relates to a group of SNP markers related to myopia or high myopia and use thereof. Specifically, the SNP sites include an SNP site combination related to myopia or high myopia, comprising: one or more of rs10484690, rs117984746, rs1196455, rs143477571, rs143580489, rs189212243, rs2296651, rs2303221, rs2547319, rs26173, rs3817585, rs41303763, rs42688, rs61746299, rs78587889, and rs80094829.

The present application claims priority to Chinese Patent Application No. 2022103884901, filed on Apr. 13, 2022, Chinese Patent Application No. 2022103869846, filed on Apr. 13, 2022, Chinese Patent Application No. 2022103869812, filed on Apr. 13, 2022, Chinese Patent Application No. 2022103869530, filed on Apr. 13, 2022, Chinese Patent Application No. 2022103869668, filed on Apr. 13, 2022, Chinese Patent Application No. 2022103884386, filed on Apr. 13, 2022, Chinese Patent Application No. 2022103885016, filed on Apr. 13, 2022, Chinese Patent Application No. 2022103884649, filed on Apr. 13, 2022, Chinese Patent Application No. 2022103869456, filed on Apr. 13, 2022, and Chinese Patent Application No. 2022105247490, filed on May 13, 2022, all of which are incorporated herein by reference in their entireties.

### TECHNICAL FIELD

The present invention belongs to the technical fields of biomedicine and gene detection, and particularly relates to a group of SNP markers related to myopia or high myopia and use thereof.

### BACKGROUND

Ophthalmic diseases refer to diseases occurring in the ocular region. Common ophthalmic diseases include myopia, astigmatism, cataract, glaucoma, central serous retinopathy, dry eye syndrome, sympathetic ophthalmia, nyctalopia, amblyopia, trachoma, diabetic retinopathy, conjunctivitis, presbyopia, color blindness, retinitis pigmentosa, central retinal artery occlusion, retinal detachment, hyperopia, hordeolum, snow blindness, chalazion, muscae volitantes, and the like. In the state of accommodating relaxation, the condition where parallel rays are focused in front of the retina after passing through the dioptric system of the eye is called myopia.

Diopter refers to the degree of power in the state of refractive error. When diopter change occurs, it usually represents the clinical manifestations such as myopia, astigmatism, hyperopia, or amblyopia. The results for myopia and hyperopia are usually represented by sphere (S), and the results for astigmatism are represented by cylinder (C). At present, the diopter is evaluated by using spherical equivalent refraction (SE), and SE=sphere+cylinder*½. More specifically, myopia is defined as the condition where the spherical equivalent refractive error of the eye is ≤−0.5D when accommodating relaxation. High myopia is defined as the condition where spherical equivalent refractive error of the eye is ≤−6.00D when accommodating relaxation.

Myopia has been receiving more and more attention in recent years at home and abroad as a global high-incidence disease. It is predicted that by 2050, myopia will affect nearly 50% of the population worldwide, and high myopia will affect nearly 10% of the population worldwide. It is of great clinical significance to find more effective methods for early detection, risk prediction, and early intervention of patients with high myopia and high-risk groups.

Single nucleotide polymorphism (snp) refers to a DNA sequence polymorphism caused by single base transition, transversion, or nucleotide variation due to base insertion and deletion on genome level, which is the most common among human heritable variations and accounts for more than 80% of all known polymorphisms, with the variation frequency in the population being higher than 1%, which is also an important factor different from point mutations. In genetic analysis, SNPs are characterized by high frequency, stability, easy analysis, and the like. With the development of technology, the cost of SNP detection becomes more and more economical, and SNP markers become a new generation of molecular markers.

### SUMMARY

In the present invention, a large population of subjects of 6-18 years old are included, and oral mucosa samples meeting the standard are collected through a standard operating procedure (SOP) to extract DNA. Then typing of single nucleotide polymorphisms (SNPs) is performed to screen for SNPs with high specificity and sensitivity that are highly related to a vision condition for the further manufacturing of a corresponding kit convenient for clinical application, which provides the data support for determining the vision condition of the subjects. In this way, interventions can be made in the early stage of the development of ophthalmic problems to reduce the possibility of ophthalmic disease deterioration and improve the life quality.

The vision condition of the present invention comprises: myopia or high myopia, spherical equivalent difference between the two eyes, spherical equivalent of the worst eye, spherical equivalent of the right eye, sphere of the right eye, cylinder of the right eye, spherical equivalent of the left eye, sphere of the left eye, and cylinder of the left eye.

As described herein, when the kit comprises a reagent for detecting the SNP site combination related to myopia or high myopia described herein, the kit is a kit for detecting myopia or high myopia; when the SNP site combination related to myopia or high myopia described herein is used for calculation in the system, the system is a system for detecting myopia or high myopia; when the model makes a determination according to a detection result for the SNP site combination related to myopia or high myopia described herein, the model is a model for determining myopia or high myopia in a subject; when the method makes a determination according to a detection result for the SNP site combination related to myopia or high myopia described herein, the method is a method for determining myopia or high myopia in a subject. The same applies to other SNP site combinations related to the following quantitative shapes: spherical equivalent difference between the two eyes, spherical equivalent of the worst eye, spherical equivalent of the right eye, sphere of the right eye, cylinder of the right eye, spherical equivalent of the left eye, sphere of the left eye, and cylinder of the left eye.

In the present invention, SNP (single nucleotide polymorphism) refers to a single base location in DNA, which may be homozygous or heterozygous in a subject. SNPs are dimorphic markers, which are caused by single base transitions or transversions, and may also be caused by base insertions or deletions. SNPs can be either within the gene sequence or on non-coding sequences outside of the gene. The SNP sites of the present invention are named in the “rs-” manner. The exact locations and nucleotide sequences of the SNP sites can be determined by those skilled in the art from a suitable database and related information systems, such as a database of single nucleotide polymorphisms (dbSNP), based on the above rs-nomenclature.

The detection of an SNP site described herein is to genotype the SNP site; more specifically, the detection of an SNP site is to determine whether the site is of a wild-type genotype, a risk-type genotype, or a heterozygous genotype (one allele is wild-type and the other is risk-type).

In order to realize the technical purpose of the present invention, the present invention provides the following technical solutions:

### SNP Site Combination

In a first aspect, the present invention provides an SNP site combination related to a vision condition, comprising any one of the following:
1) an SNP site combination related to myopia or high myopia, comprising: one or more of rs10484690, rs117984746, rs1196455, rs143477571, rs143580489, rs189212243, rs2296651, rs2303221, rs2547319, rs26173, rs3817585, rs41303763, rs42688, rs61746299, rs78587889, and rs80094829;
2) an SNP site combination related to spherical equivalent difference between the two eyes;
3) an SNP site combination related to spherical equivalent of the worst eye;
4) an SNP site combination related to spherical equivalent of the right eye;
5) an SNP site combination related to sphere of the right eye;
6) an SNP site combination related to cylinder of the right eye;
7) an SNP site combination related to spherical equivalent of the left eye;
8) an SNP site combination related to sphere of the left eye; and
9) an SNP site combination related to cylinder of the left eye.

In the present invention, the SNP site combination related to spherical equivalent difference between the two eyes comprises a first SNP site combination related to spherical equivalent difference between the two eyes, a second SNP site combination related to spherical equivalent difference between the two eyes, a third SNP site combination related to spherical equivalent difference between the two eyes, and a fourth SNP site combination related to spherical equivalent difference between the two eyes.

In the present invention, the SNP site combination related to spherical equivalent of the worst eye comprises a first SNP site combination related to spherical equivalent of the worst eye, a second SNP site combination related to spherical equivalent of the worst eye, a third SNP site combination related to spherical equivalent of the worst eye, and a fourth SNP site combination related to spherical equivalent of the worst eye.

In the present invention, the SNP site combination related to spherical equivalent of the right eye comprises a first SNP site combination related to spherical equivalent of the right eye, a second SNP site combination related to spherical equivalent of the right eye, and a third SNP site combination related to spherical equivalent of the right eye.

In the present invention, the SNP site combination related to sphere of the right eye comprises a first SNP site combination related to sphere of the right eye, a second SNP site combination related to sphere of the right eye, and a third SNP site combination related to sphere of the right eye.

In the present invention, the SNP site combination related to cylinder of the right eye comprises a first SNP site combination related to cylinder of the right eye, a second SNP site combination related to cylinder of the right eye, a third SNP site combination related to cylinder of the right eye, and a fourth SNP site combination related to cylinder of the right eye.

In the present invention, the SNP site combination related to spherical equivalent of the left eye comprises a first SNP site combination related to spherical equivalent of the left eye, a second SNP site combination related to spherical equivalent of the left eye, and a third SNP site combination related to spherical equivalent of the left eye.

In the present invention, the SNP site combination related to sphere of the left eye comprises a first SNP site combination related to sphere of the left eye, a second SNP site combination related to sphere of the left eye, and a third SNP site combination related to sphere of the left eye.

In the present invention, the SNP site combination related to cylinder of the left eye comprises a first SNP site combination related to cylinder of the left eye, a second SNP site combination related to cylinder of the left eye, a third SNP site combination related to cylinder of the left eye, and a fourth SNP site combination related to cylinder of the left eye.

The first SNP site combination related to spherical equivalent difference between the two eyes described herein comprises: rs80094829, rs1196455, rs61746299, rs3817585, and rs2296651.

The second SNP site combination related to spherical equivalent difference between the two eyes described herein comprises: one or more of rs138802237, rs80094829, rs1196455, rs61746299, rs17846715, rs77819192, rs74711761, rs186128635, rs3817585, rs166508, rs11745612, rs164074, rs164075, rs164076, rs140779761, rs17652080, rs17708515, rs17110944, rs140691873, rs1042454, rs1613776, rs57344881, rs2296651, rs2302230, rs2302231, rs4147910, rs74406853, rs199946960, rs35689937, rs34378744, rs71357953, rs71357954, rs201326575, and rs2075937.

The third SNP site combination related to spherical equivalent difference between the two eyes described herein comprises: one or more of rs183232121, rs138802237, rs7539625, rs80094829, rs1196455, rs61746299, rs150026164, rs12043009, rs17846715, rs4653415, rs77819192, rs117238208, rs34117315, rs4851287, rs74711761, rs13422424, rs186128635, rs150129946, rs35678, rs2289272, rs3817585, rs166508, rs73135528, rs73135529, rs73135530, rs73135535, rs73135545, rs36077176, rs73135584, rs137919012, rs3813832, rs11745612, rs56969869, rs397741115_rs56191507, rs16899974, rs164073, rs164074, rs164075, rs164076, rs140779761, rs17652080, rs17708515, rs17110944, rs6627, rs61748571, rs137899339, rs117192504, rs1109208, rs73689406, rs192085105, rs1830035, rs1830036, rs17138865, rs17545405, rs140691873, rs17553698, rs3810925, rs111748634, rs111841972, rs4073310, rs2271460, rs1042454, rs1044476, rs2274351, rs1613776, rs57344881, rs76274604, rs694066, rs7123133, rs7123272, rs28734900, rs12797894, rs17102543, rs1050395, rs181464388, rs2296651, rs3818188, rs1455773, rs13380006, rs199816463, rs429744, rs370627229, rs3743600, rs138793855, rs35896669, rs2302230, rs2302231, rs2907373, rs116067668, rs138221111, rs3752229, rs3764648, rs4147910, rs4147914, rs80054150, rs150907742, rs200222185, rs74406853, rs199946960, rs35689937, rs34378744, rs6511379, rs71357953, rs71357954, rs28678477, rs201326575, rs874100, rs1534879, rs2075937, and rs2239749.

The fourth SNP site combination related to spherical equivalent difference between the two eyes described herein comprises: one or more of rs12133253, rs1573753, rs7512414, rs34109355, rs34976233, rs35013594, rs7528572, rs1541185, rs146160422, rs41303641, rs41311274, rs183232121, rs75321043, rs79777494, rs138802237, rs7664, rs1884444, rs7539625, rs76475726, rs599839, rs12083584, rs374505350, rs80094829, rs1196455, rs61746299, rs200519781, rs150026164, rs12043009, rs1375211271_rs373796693, rs17846707, rs17846715, rs3747626, rs1301522897_rs397775888, rs17846717, rs17846718, rs41288127, rs12087593, rs17380127, rs17380141, rs77202104, rs4653415, rs77819192, rs117238208, rs28383065, rs3731572, rs4148186, rs151242484, rs34117315, rs370613331, rs4851287, rs144642601, rs200736867, rs74711761, rs13422424, rs185464655, rs148663631, rs186128635, rs79028939, rs150129946, rs236843, rs10173558, rs150979437, rs140446783, rs56847463, rs183596892, rs117354149, rs35678, rs2075354, rs2289272, rs3817585, rs166508, rs370554533, rs78946357, rs73135513, rs73135528, rs73135529, rs73135530, rs73135535, rs73135545, rs36077176, rs73135584, rs117279588, rs137919012, rs17552950, rs151025946, rs77422573, rs3813832, rs4752, rs77676024, rs7687613, rs74991481, rs77885682, rs13152799, rs2276979, rs11745612, rs113222960, rs56969869, rs12523335, rs397741115_rs56191507, rs16899974, rs2560306, rs186414752, rs351260, rs164073, rs164074, rs164075, rs164076, rs140779761, rs17652080, rs17708515, rs17110944, rs111995800, rs3839291_rs398000092, rs6627, rs17136239, rs61748571, rs2815822, rs35374726, rs77229316, rs3094225, rs2281831, rs113689068, rs137899339, rs17710008, rs7763726, rs117192504, rs186624179, rs2292498, rs3763384, rs146123998, rs17779352, rs192189247, rs1109208, rs73689406, rs192085105, rs1830035, rs1830036, rs17138865, rs17545405, rs41350445, rs34587586, rs78637678, rs4503083, rs188235566, rs140691873, rs79930604, rs41297187, rs41307479, rs17553698, rs3810925, rs111748634, rs111841972, rs7850697, rs2231406, rs4073310, rs2271460, rs80042909, rs11200920, rs11200933, rs1042454, rs1934951, rs1044476, rs11191295, rs7081110, rs7081467, rs2274351, rs1613776, rs3135802, rs6693, rs183813046, rs57344881, rs907610, rs80326119, rs76274604, rs77600363, rs486584, rs12804382, rs12805133, rs201373803, rs694066, rs189385634, rs149080490, rs138619667, rs7123133, rs7123272, rs28734900, rs12797894, rs7307854, rs76066543, rs139938131, rs7980396, rs3217157_rs35118302_rs537549359, rs149247686, rs75656873, rs2277373, rs59261129, rs181052973, rs183437347, rs17102543, rs1050395, rs3751129, rs77973991, rs2036771, rs4759544, rs181464388, rs112163489, rs9594738, rs3210286, rs191708663, rs149954510, rs440466, rs2331979, rs7159367, rs34598192, rs2277475, rs3729818, rs117109392, rs58154675, rs2295106, rs2295107, rs2235967, rs7143196, rs2296651, rs116897063, rs562694521, rs536155089, rs1884541, rs3818188, rs200138830, rs7177192, rs2412541, rs11858113, rs8041534, rs8040502, rs11070285, rs2412544, rs141891738, rs12232341, rs143563919, rs3743476, rs1455773, rs13380006, rs74642800, rs199816463, rs373684171, rs429744, rs370627229, rs78671991, rs529955941, rs41303763, rs11867123, rs7206481, rs3743600, rs11656203, rs138793855, rs115332545, rs3736238, rs147085136, rs11649834, rs140947135, rs8556, rs2303015, rs3826301, rs35896669, rs75743672, rs729782, rs59589773, rs184602743, rs2302230, rs2302231, rs2907373, rs140010427, rs3744032, rs2748428, rs73410309, rs11657217, rs148660676, rs1192271300_rs397858239, rs566655, rs116067668, rs138221111, rs189222917, rs375483921, rs3752229, rs3764648, rs4147910, rs4147914, rs1610096, rs2074442, rs117187726, rs2074454, rs80054150, rs150907742, rs4807578, rs143662386, rs28480458, rs139113505, rs200222185, rs74406853, rs199946960, rs35689937, rs34378744, rs6511379, rs71357953, rs71357954, rs1801015, rs80027112, rs4635, rs56276635, rs184733141, rs79863139, rs181599488, rs138399909, rs324125, rs8109631, rs80087697, rs200619037, rs28678477, rs2273423, rs6023533, rs5743513, rs817343, rs45566234, rs7281104, rs14194, rs201326575, rs874100, rs202195284, rs1894473, rs17670808, rs17738527, rs17738540, rs9606738, rs9608944, rs9608956, rs1534879, rs2075937, rs2239749, rs202112368, rs188655025, and rs11541025.

The first SNP site combination related to spherical equivalent of the worst eye described herein comprises: one or more of rs78587889, rs41303763, and rs2303221.

The second SNP site combination related to spherical equivalent of the worst eye described herein comprises: one or more of rs78587889, rs188480146, rs2292661, rs6894260, rs4957836, rs148474510, rs200506987, rs145293869, rs2248077, rs2811736, rs2297722, rs2297723, rs9987876, rs7157977, rs41303763, rs2303221, rs145438063, rs151309111, rs6076122, rs2183573, rs9981301, and rs5756223.

The third SNP site combination related to spherical equivalent of the worst eye described herein comprises: one or more of rs3103778, rs2746342, rs78587889, rs140141416, rs17024525, rs2288182, rs188480146, rs6734083, rs75054661, rs12492484, rs2292661, rs55947930, rs16837029, rs149213974, rs6894260, rs45625534, rs4957836, rs147518598, rs1042044, rs12671595, rs186526946, rs138414411, rs3802064, rs75456709, rs76427362, rs2073793, rs140518317, rs2214098, rs148474510, rs876087, rs200506987, rs8178175, rs145293869, rs200021348_rs552590290, rs16909677, rs2248077, rs2811736, rs2297722, rs2297723, rs9987876, rs34630110, rs141296329, rs117520659, rs3750996, rs3847553, rs142908193, rs1042669, rs12811832, rs78692679, rs11551723, rs7157977, rs3759779, rs3742770, rs530110551, rs3742464, rs3736911, rs11849022, rs77481241, rs3212112, rs9282879, rs55958706, rs3212056, rs114770841, rs3742368, rs563363438, rs2738893, rs1132358, rs41303763, rs2303221, rs145438063, rs151309111, rs139271842, rs533816037, rs305974, rs407731, rs406968, rs388706, rs2302948, rs2547319, rs7258847, rs4815467, rs41275624, rs6057110, rs6076122, rs4911402, rs2183573, rs9981301, rs4624474, rs5756223, and rs5845912.

The fourth SNP site combination related to spherical equivalent of the worst eye described herein comprises: one or more of rs3795289, rs17033266, rs61785839, rs77649572, rs202140107, rs2275365, rs77275487, rs6676052, rs3103778, rs3014246, rs3014240, rs35016075_rs59669671, rs28375469, rs116861599, rs2746342, rs2457829, rs10889315, rs148811681, rs41305862, rs1410871, rs61729628, rs17553619, rs78587889, rs140141416, rs17024525, rs149097658, rs822441, rs183812847, rs112946310, rs12568050, rs12564283, rs75401237, rs10749643, rs2288182, rs400917, rs201752014, rs75526749, rs17189743, rs188480146, rs528123098, rs61748134, rs2228184, rs143686122, rs1434087, rs13002576, rs192688643, rs6734083, rs68104327, rs35996697, rs6437368, rs1077827, rs76657333, rs75054661, rs547922447, rs143654927, rs2286786, rs115250058, rs117336796, rs12492484, rs2292661, rs2276740, rs3774765, rs2245278, rs55947930, rs75144949, rs3732755, rs16837029, rs11732898, rs7696197, rs6841898, rs114264770, rs75058604, rs149213974, rs17001890, rs61732380, rs17029087, rs76996680, rs186309103, rs2303710, rs3749692, rs181303223, rs150821721, rs76381196, rs139298086_rs17839482, rs555615570, rs6894260, rs13189822, rs45625534, rs4957836, rs147518598, rs3829986, rs4608967, rs564109, rs150157174, rs2735059, rs4959012, rs757262, rs757259, rs117333063, rs185990098, rs41542812, rs11966699, rs4711571, rs4711572, rs2235868, rs1042044, rs3804502, rs2100375, rs118003870, rs75817012, rs17181352, rs35517174, rs17132399, rs2292498, rs3763384, rs12671595, rs186526946, rs138414411, rs13438494, rs3802064, rs75456709, rs76427362, rs2073793, rs140518317, rs2214098, rs79413133, rs2230156, rs148474510, rs41465145, rs876087, rs200506987, rs537284787, rs8178175, rs55991828, rs12549018, rs34961221, rs78240711, rs139669465, rs11144089, rs117169590, rs145293869, rs200021348_rs552590290, rs16909677, rs117175949, rs10481674, rs41276795, rs141549766, rs147026086, rs188197626, rs7038042, rs11849, rs2275161, rs10781513, rs2248077, rs2811736, rs2297722, rs2297723, rs9987876, rs7079747, rs34630110, rs148582275, rs4745933, rs111835023, rs185249748, rs117768118, rs141296329, rs117520659, rs45498396, rs3750996, rs183484, rs9937, rs2304733, rs147852038, rs139692587, rs10792769, rs2298573, rs4753069, rs3847553, rs2962118, rs142908193, rs1042669, rs11047401, rs12811832, rs11168338, rs2277373, rs1150057, rs1799986, rs1050395, rs78692679, rs3835190, rs201814780, rs11551723, rs7992732, rs7157977, rs10143899, rs3759779, rs7142098, rs118004333, rs3742770, rs3742768, rs3742767, rs143477571, rs530110551, rs3742464, rs3736911, rs11849022, rs77481241, rs3212112, rs9282879, rs55958706, rs3212056, rs34100926, rs114770841, rs3742368, rs4390564, rs72486624, rs7172888, rs563363438, rs12904657, rs4904, rs72546313, rs2745136, rs2738893, rs1054645, rs1132358, rs79763179, rs3743807, rs55969568, rs9941128, rs41303763, rs2303221, rs11859725, rs113276760, rs145438063, rs11550470, rs145797873, rs11556634, rs56126318, rs16954357, rs397718340_rs5819492, rs9899785, rs375644567, rs185576254, rs183898893, rs4986765, rs146684394, rs181969864, rs2242273, rs2170297, rs11081410, rs7233770, rs7235093, rs7236574, rs11659762, rs116067668, rs11538683, rs55646937, rs148018996, rs1143698, rs10412834, rs11260004, rs11882197, rs2288415, rs150461309, rs3746295, rs151309111, rs3810209, rs4808643, rs139271842, rs200222185, rs189120571, rs148003617, rs533816037, rs189041504, rs305968, rs305974, rs407731, rs406968, rs388706, rs2302948, rs182509214, rs198977, rs2547319, rs2547318, rs7258847, rs4815467, rs41275624, rs6057110, rs6076122, rs4911402, rs114985890, rs80158178, rs3746488, rs76747058, rs143199162, rs77432560, rs116846998, rs2183573, rs1984748, rs9981301, rs4624474, rs2838379, rs4566449, rs915770, rs2870984, rs2073733, rs41281587, rs3747135, rs3747136, rs3747137, rs5756223, rs3747168, rs369859899, rs6006901, rs5845912, rs9628315, and rs8137790.

The first SNP site combination related to spherical equivalent of the right eye described herein comprises: one or more of rs10889315, rs75144949, rs149213974, rs754615, rs45583335, rs2529438, rs2072236, rs2240403, rs78155900, rs114097201, rs2811736, rs2297722, rs2297723, rs117519669, rs7157977, rs10143899, rs3759779, rs143477571, rs2303221, rs34693726, rs3803752, rs142921938, rs3746295, rs2547319, rs2547318, and rs4911402.

The second SNP site combination related to spherical equivalent of the right eye described herein comprises: one or more of rs4648600, rs1061624, rs2275365, rs35909785, rs3103778, rs2457829, rs10889315, rs78587889, rs12568050, rs12564283, rs528123098, rs139842833, rs74953908, rs1077827, rs12492484, rs75144949, rs3732755, rs3135115, rs16837029, rs149213974, rs17001890, rs56382032, rs12651031, rs2736100, rs7724759, rs754615, rs5369, rs150157174, rs130068, rs45583335, rs78342561, rs2529438, rs1049402, rs2072236, rs2527878, rs2240403, rs78155900, rs114097201, rs2680903, rs59612871, rs3818584, rs16909677, rs10983755, rs2248077, rs2811736, rs2297722, rs2297723, rs9987876, rs7904554, rs907609, rs117519669, rs3750996, rs3802799, rs2276296, rs139692587, rs904628, rs11551723, rs144796593, rs78648016, rs7157977, rs10143899, rs3759779, rs7142098, rs143477571, rs3736911, rs11849022, rs77481241, rs3212112, rs9282879, rs55958706, rs3212056, rs34100926, rs114770841, rs3742368, rs118146919, rs2289702, rs2303221, rs34693726, rs3744566, rs9907420, rs375644567, rs185576254, rs3803752, rs17822735, rs17222523, rs34818467, rs12946397, rs3809724, rs181969864, rs7236574, rs142921938, rs3746295, rs151309111, rs533816037, rs2302948, rs182509214, rs2547319, rs2547318, rs2281209, rs4911402, rs138894461, and rs5756223.

The third SNP site combination related to spherical equivalent of the right eye described herein comprises: one or more of rs16824585, rs4648600, rs17033266, rs1061624, rs202140107, rs2291804, rs2275365, rs139241751, rs35909785, rs6676052, rs3103778, rs116893711, rs116861599, rs2457829, rs10889315, rs17855078, rs78587889, rs140141416, rs4656197, rs45445497, rs143355122, rs12568050, rs12564283, rs10482, rs1929861, rs75401237, rs28517482, rs1264208208, rs2631840, rs188480146, rs528123098, rs61732269, rs367931009, rs139842833, rs79908358, rs79358798, rs1434087, rs4894048, rs13421990, rs13419085, rs143196218, rs139713392, rs6734083, rs150887565, rs4663795, rs74953908, rs143001518, rs6437368, rs147838565, rs1077827, rs144623840, rs78233496, rs9876921, rs6622, rs2279909, rs547922447, rs300977, rs12492484, rs75144949, rs3732755, rs3135115, rs16837029, rs74787220, rs527903313, rs149213974, rs17001890, rs79769348, rs10025654, rs76996680, rs2306369, rs79482316, rs56382032, rs12651031, rs2736100, rs35130836, rs3749684, rs7724759, rs754615, rs45625534, rs3805625, rs137898880, rs147518598, rs202007699, rs2270900, rs75164992, rs17133512, rs76720430, rs5369, rs150157174, rs7756481, rs117298661, rs117644703, rs3094672, rs4713420, rs130068, rs67196728, rs185990098, rs9349593, rs143580489, rs3757302, rs3218602, rs9495370, rs45583335, rs78342561, rs117358940, rs9689983, rs2529438, rs1049402, rs2072236, rs2527878, rs2240403, rs781980620, rs56083327, rs2245368, rs2430307, rs3748126, rs36028884, rs78155900, rs114097201, rs13438494, rs117211799, rs115734214, rs148474510, rs200506987, rs8190955, rs8178175, rs2680903, rs117676215, rs115507207, rs369806130, rs78240711, rs59612871, rs117169590, rs3739523, rs145293869, rs139212809, rs3818584, rs16909677, rs141549766, rs147026086, rs188197626, rs13321, rs10983755, rs2301576, rs7038042, rs11849, rs2275161, rs2248077, rs2811736, rs2297722, rs2297723, rs9987876, rs7904554, rs148582275, rs3740168, rs149647444, rs7100474, rs7079648, rs202166096, rs60420182, rs36104328, rs7098255, rs3814163, rs2297785, rs3736924, rs3736923, rs141296329, rs117520659, rs57285449, rs112687793, rs73401681, rs79948463, rs10902170, rs10902171, rs867839, rs907608, rs907609, rs117519669, rs3750996, rs214086, rs3802799, rs76215382, rs147852038, rs2276296, rs143930161, rs183113103, rs139692587, rs10792769, rs7102675, rs116878689, rs35466364, rs142908193, rs7966459, rs7135745, rs34181394, rs1921038, rs201814780, rs11068531, rs904628, rs11551723, rs7983667, rs144796593, rs140526815_rs77878407, rs78646508, rs34523940, rs9521906, rs78648016, rs7157977, rs10143899, rs3759779, rs7142098, rs41309252, rs535020964, rs3742770, rs3742768, rs116881452, rs143477571, rs61992274, rs142973720, rs530110551, rs3742464, rs3736911, rs11849022, rs77481241, rs3212112, rs9282879, rs55958706, rs3212056, rs34100926, rs114770841, rs3742368, rs144857261_rs373032548, rs189734192, rs188393827, rs118146919, rs562829514, rs1805025, rs8192352, rs2289702, rs150921132, rs61869621, rs1802752, rs113355794, rs7198064, rs2291407, rs9941128, rs9941210, rs9941215, rs9931527, rs12708649, rs13306653, rs11643517, rs5723, rs7196736, rs2303221, rs2304483, rs149645127, rs11550470, rs17232091, rs150889000, rs16954357, rs371756524_rs557159990, rs192103504, rs115314145, rs34693726, rs2277651, rs2277652, rs71360269, rs71360270, rs3744566, rs3744568, rs9907420, rs375644567, rs185576254, rs2158460, rs3803752, rs17822735, rs17222523, rs34818467, rs12946397, rs3809724, rs35476409_rs397978887, rs3744214, rs181969864, rs4789773, rs28561791, rs7236574, rs76597875, rs61749945, rs118033234, rs1052696, rs1052692, rs148018996, rs34488963, rs3745379, rs3745385, rs142921938, rs3746295, rs151309111, rs139271842, rs140566569, rs533816037, rs78237760, rs79621739, rs75990051, rs62107869, rs305974, rs1055099, rs41290128, rs76151738, rs16980091, rs2302948, rs182509214, rs2547319, rs2547318, rs16982743, rs2284385, rs2281209, rs4911402, rs80158178, rs7509972, rs150511237, rs2231391, rs2231390, rs762228, rs72564613, rs191574093, rs138894461, rs5999924, rs5750146, rs5756223, rs3747168, rs148345118, rs910796, rs910797, rs910798, rs738712, rs16445, rs3922872, rs56248708, rs9628315, and rs8137790.

The first SNP site combination related to sphere of the right eye described herein comprises: one or more of rs1929861, rs139842833, rs149213974, rs130068, rs2072236, rs2240403, rs59612871, rs139692587, rs143477571, rs2303221, rs34693726, rs3744566, rs3803752, rs17223038, rs34488963, rs142921938, and rs2547319.

The second SNP site combination related to sphere of the right eye described herein comprises: one or more of rs35909785, rs10889315, rs143355122, rs10482, rs1929861, rs191149396, rs528123098, rs139842833, rs12492484, rs75144949, rs3732755, rs3135115, rs16837029, rs149213974, rs79769348, rs55656741, rs7724759, rs754615, rs137898880, rs147518598, rs75164992, rs17133512, rs76720430, rs3094672, rs4713420, rs130068, rs286560, rs2529438, rs2072236, rs2527878, rs2240403, rs56083327, rs2245368, rs2430307, rs3748126, rs36028884, rs117676215, rs115507207, rs2403730, rs59612871, rs3739523, rs2811736, rs2297722, rs2297723, rs202166096, rs2297785, rs3736924, rs3736923, rs117519669, rs57477011, rs139692587, rs10792769, rs142908193, rs140526815 (same as rs77878407), rs78646508, rs34523940, rs9521906, rs78648016, rs143477571, rs3742464, rs3736911, rs11849022, rs77481241, rs3212112, rs9282879, rs55958706, rs3212056, rs34100926, rs114770841, rs3742368, rs189734192, rs9941210, rs9941215, rs12708649, rs13306653, rs11643517, rs5723, rs2303221, rs11550470, rs2115400, rs150889000, rs192103504, rs2024076, rs34693726, rs2277648, rs2286357, rs12950718, rs2277651, rs2277652, rs71360269, rs71360270, rs3744566, rs3744568, rs3803752, rs17822735, rs17222523, rs34818467, rs12946397, rs3809724, rs3744214, rs7236574, rs1064058, rs1064059, rs17223038, rs34488963, rs3745379, rs3745385, rs142921938, rs3746295, rs2302948, rs2547319, rs2547318, rs4911402, and rs191574093.

The third SNP site combination related to sphere of the right eye described herein comprises: one or more of rs16824585, rs4648600, rs10864435, rs1061624, rs76538441, rs35909785, rs587404, rs3103778, rs10889315, rs17855078, rs78587889, rs140141416, rs117378923, rs143355122, rs12568050, rs12564283, rs10482, rs1929861, rs2275307, rs1339907598, rs115095929, rs191149396, rs1264208208, rs574401502, rs11558179, rs2303607, rs528123098, rs61732269, rs367931009, rs3796110, rs139842833, rs79908358, rs74765462, rs57399379, rs143196218, rs139713392, rs28900695, rs74953908, rs147838565, rs125701, rs1801126, rs142876339, rs1969154, rs1985428, rs186408122, rs144623840, rs148545377, rs3749195, rs9876921, rs6622, rs2279909, rs724450, rs1138518, rs12492484, rs183217537, rs150448043, rs75144949, rs3732755, rs9857697, rs3135115, rs16837029, rs117253156, rs527903313, rs149213974, rs79769348, rs76996680, rs3828506, rs56382032, rs117171059, rs2736100, rs55656741, rs181303223, rs35130836, rs7724759, rs754615, rs137898880, rs147518598, rs202007699, rs13184586, rs2270900, rs2270898, rs75164992, rs17133512, rs139302155, rs76720430, rs5369, rs150157174, rs117298661, rs117644703, rs3094672, rs4713420, rs130068, rs9349593, rs2754779, rs75817012, rs45583335, rs78342561, rs117358940, rs78873069, rs286560, rs9689983, rs2529438, rs1049402, rs2072236, rs2527878, rs2240403, rs781980620, rs56083327, rs2245368, rs2430307, rs3748126, rs36028884, rs78155900, rs114097201, rs13438494, rs117211799, rs115734214, rs11556986, rs361330, rs2294127, rs2680903, rs117676215, rs115507207, rs114091176, rs11778661, rs2403730, rs7023474, rs78240711, rs149966849, rs139485366, rs59612871, rs117169590, rs3739523, rs200021348, rs2274689, rs28446339, rs41306498, rs972925, rs2248077, rs2811736, rs2297722, rs2297723, rs9987876, rs7904554, rs148582275, rs7900977, rs35963845, rs11239594, rs202166096, rs60420182, rs140715254, rs2297785, rs3736924, rs3736923, rs117520659, rs907608, rs907609, rs117519669, rs16911845, rs3802799, rs662702, rs667773, rs57477011, rs61744209, rs76215382, rs147852038, rs2736597, rs1800007, rs143930161, rs183113103, rs139692587, rs10792769, rs2298573, rs7102675, rs56252953, rs116878689, rs10893102, rs74550564, rs35466364, rs215976, rs142908193, rs7966459, rs1921038, rs117866676, rs11551723, rs140728821, rs7983667, rs144796593, rs140526815, rs78646508, rs34523940, rs9521906, rs3211770, rs78648016, rs7157977, rs10143899, rs3759779, rs7142098, rs116881452, rs143477571, rs192594531, rs530110551, rs3742464, rs3736911, rs11849022, rs77481241, rs3212112, rs9282879, rs55958706, rs3212056, rs34100926, rs114770841, rs3742368, rs189734192, rs188393827, rs185222838, rs780162072, rs28381099, rs147960756, rs62032959, rs62033860, rs1802752, rs113355794, rs74988150, rs76534191, rs79609879, rs7198064, rs2291407, rs79763179, rs9941128, rs9941210, rs9941215, rs9931527, rs12708649, rs13306653, rs11643517, rs5723, rs7196736, rs71220135, rs202213258, rs2303221, rs117161621, rs11540994, rs149645127, rs117127365, rs13339651, rs11550470, rs2162943, rs2115400, rs4247353, rs3803690, rs3751696, rs75670228, rs150889000, rs16954357, rs192103504, rs2024076, rs12936716, rs34693726, rs2277648, rs2277649, rs12949680, rs2286357, rs12950718, rs2277651, rs2277652, rs71360269, rs71360270, rs3744566, rs3744568, rs4792008, rs2285479, rs2285475, rs2285474, rs2285473, rs2285472, rs2285471, rs2285469, rs876660, rs9907420, rs1049807, rs17843022, rs183898893, rs145872131, rs139515721, rs2158460, rs3803752, rs17822735, rs17222523, rs34818467, rs12946397, rs3809724, rs35476409, rs3744214, rs3744807, rs181969864, rs4789773, rs28561791, rs28365945, rs28365946, rs2242273, rs2170297, rs11081410, rs7233770, rs7235093, rs4798691, rs7236574, rs1064058, rs1064059, rs3744878, rs1050919, rs17223038, rs117801197, rs118033234, rs76914699, rs2304191, rs148018996, rs34488963, rs3745379, rs3745385, rs141221917, rs4926171, rs142921938, rs3746295, rs151309111, rs139271842, rs115266843, rs143479651, rs140566569, rs533816037, rs75935970, rs741143, rs3746013, rs3855679, rs62107869, rs1055099, rs41290128, rs76151738, rs2302948, rs2547319, rs2547318, rs146619376, rs2284385, rs2281209, rs4911402, rs369156308, rs17333103, rs34885285, rs17424474, rs17333381, rs2233882, rs2233883, rs143199162, rs6027195, rs7509972, rs150511237, rs191574093, rs138894461, rs538007588, rs79018232, rs5999924, rs5750146, rs5756223, rs3747168, rs148345118, rs2076213, and rs6006901.

The first SNP site combination related to cylinder of the right eye described herein comprises: one or more of rs42688, rs143580489, rs10484690, and rs189212243.

The second SNP site combination related to cylinder of the right eye described herein comprises: one or more of rs12408385, rs2275365, rs374309842, rs77901289, rs147707514, rs143100530, rs41298993, rs26173, rs26098, rs1353251, rs5867289, rs1494555, rs42688, rs32210, rs143580489, rs1204114, rs10484690, rs118151455, rs201920957, rs189212243, rs117569740, rs2271001, rs185257411, rs698028, rs4923849, rs12904657, rs62037369, rs13337155, rs17223038, rs199667693, and rs148965154.

The third SNP site combination related to cylinder of the right eye described herein comprises: one or more of rs12408385, rs2275365, rs2275360, rs368345074, rs80002732, rs374309842, rs77901289, rs191506876, rs147707514, rs3087649, rs10177030, rs143100530, rs1434087, rs3748962, rs28900693, rs1077827, rs80341144, rs3213930, rs41298993, rs3749584, rs10517195, rs2271924, rs13147012, rs79482316, rs3827497, rs26173, rs26098, rs26107, rs26086, rs1353251, rs5867289, rs1494555, rs42688, rs32210, rs154483, rs7764126, rs13205210, rs143580489, rs1204114, rs10484690, rs118151455, rs200111157, rs113281844, rs201920957, rs79960636, rs112596877, rs117509972, rs189212243, rs74699763, rs2584, rs7023474, rs117569740, rs147367410, rs61748723, rs17849182, rs79735301, rs145978705, rs304494, rs115526095, rs7907476, rs4880361, rs2271001, rs111885243, rs2074233, rs622082, rs546382, rs185257411, rs145509540, rs144650014, rs17103265 (same as rs397844079), rs11570779, rs17793018, rs76846944, rs698028, rs4923849, rs12904657, rs72765429, rs111980103, rs61737565, rs4788099, rs7198606, rs62037369, rs62037371, rs10499, rs13337155, rs61742982, rs9303742, rs17223038, rs199667693, rs148965154, rs706764, rs56408444, rs117753855, rs78472358, rs6037509, rs150212573, rs147911723, and rs470101.

The fourth SNP site combination related to cylinder of the right eye described herein comprises: one or more of rs3795262, rs12409277, rs61735125, rs61746709, rs191051279, rs151112364, rs2291804, rs12408385, rs2275365, rs2275360, rs146696696, rs11542470, rs12386, rs74622002, rs368345074, rs142732858, rs80002732, rs374309842, rs144649977, rs78780269, rs2275863, rs77901289, rs191506876, rs117369152, rs147707514, rs6715296, rs3087649, rs7584256, rs10177030, rs11694145, rs4149520, rs143100530, rs1434087, rs77497147, rs2004888, rs11568351, rs141302000, rs3748962, rs11574750, rs16858808, rs16858811, rs141559295, rs3769838, rs6734083, rs28900693, rs4663795, rs34401965, rs76999397, rs11919775, rs1869852, rs1869851, rs1077827, rs80341144, rs3213930, rs2029618, rs75318799, rs11719458, rs33988592, rs79845192, rs41298993, rs2124541, rs66748594, rs28372853, rs572169, rs2246901, rs73189480, rs3733217, rs34611001, rs61746000, rs117486807, rs147434354, rs35762546, rs3749584, rs2271036, rs2271037, rs10517195, rs41498446, rs3733324, rs2271924, rs1801612, rs2280099, rs8582, rs13147012, rs79482316, rs3827497, rs26173, rs26098, rs26107, rs26086, rs1051489, rs2270915, rs3813474, rs1353252, rs1353251, rs1494561, rs1353250, rs5867289, rs1494558, rs1494555, rs987106, rs2233216, rs2233213, rs3217552 (same as rs397840383), rs16874505, rs3749684, rs3729668, rs28388868, rs144533176, rs42688, rs32210, rs13180243, rs154483, rs367805, rs2631368, rs117231680, rs10053292, rs7701410, rs11249552, rs142883877, rs150856424, rs3734391, rs9370867, rs77229316, rs17194565, rs117487649, rs1264319, rs35756329, rs17201248, rs190135313, rs7764126, rs11755393, rs13205210, rs3734264, rs1051115, rs74609186, rs6928630, rs143580489, rs1204114, rs10484690, rs118151455, rs143904280, rs9459355, rs9459356, rs113806172, rs10227750, rs200111157, rs539973087, rs182692351, rs13306387, rs79809732, rs113281844, rs201920957, rs236652, rs236655, rs236666, rs79960636, rs112596877, rs117509972, rs772373052, rs801841, rs1062394, rs200333762, rs4301376, rs775410128, rs3987896, rs146160185, rs17171113, rs2788473, rs189212243, rs74699763, rs2584, rs41488144, rs13306550, rs77061383, rs145349678, rs371193886, rs8178175, rs4925828, rs7023474, rs3802392, rs4879781, rs35066989, rs112858544, rs1164863576, rs16909677, rs117738198, rs117569740, rs147367410, rs61748723, rs16916100, rs2297530, rs77309503, rs77564189, rs371047734, rs17849182, rs79735301, rs145978705, rs118012211, rs76191089, rs142547455, rs2073878, rs78774780, rs140120230, rs150198392, rs10600924, rs7921838, rs3829125, rs183963660, rs17134592, rs569927230, rs41382649, rs11255267, rs144121793, rs12773592, rs139646798, rs7095891, rs11003124, rs202046718, rs7076156, rs79298145, rs3758464, rs16935560, rs304494, rs304439, rs116994405, rs11553577, rs61731067, rs7100644, rs115526095, rs7907476, rs2901895, rs7912552, rs11017735, rs4880361, rs3764896, rs1056819, rs729662, rs148238084, rs138312423, rs2271001, rs111885243, rs6485702, rs326222, rs2013867, rs901746, rs2167079, rs7120118, rs1050922, rs2074233, rs77749149, rs78038516, rs11231726, rs12146487, rs182061168, rs514076, rs145270731, rs3741172, rs1551886, rs17856219, rs1551888, rs622082, rs546382, rs488792, rs568862, rs187530720, rs2276278, rs2846412, rs143351376, rs117176605, rs10844777, rs1948516, rs2232566, rs7132546, rs12313730, rs186809868, rs35988455 (same as rs67266718), rs2178685, rs73218270, rs11547907, rs9658501, rs3914, rs555404, rs10773127, rs185257411, rs145509540, rs9315569, rs953958, rs953957, rs2183207, rs4578496, rs4614565, rs151310875, rs377238277, rs144650014, rs2274827, rs17103265 (same as rs397844079), rs11570779, rs12883882, rs17793018, rs76846944, rs2297298, rs698028, rs944050, rs2069590, rs780162072, rs73370219, rs4923849, rs12904657, rs3068, rs1046657, rs12324091, rs3743267, rs12438118, rs556233670, rs149146765, rs193114293, rs2227933, rs3214113 (same as rs397897626 or rs886038220), rs72765429, rs34855511, rs400037, rs387467, rs111980103, rs367960426, rs182015467, rs117824484, rs373109045, rs41303763, rs149271, rs180743, rs180744, rs151174, rs181206, rs61737565, rs4788099, rs7198606, rs62037369, rs62037371, rs9931989, rs10499, rs140411458 (same as rs35784618), rs71220135, rs13337155, rs141238615, rs1055708, rs114991387, rs145160385, rs4796407, rs77805790, rs756223, rs61742982, rs117908897, rs9890678, rs11080105, rs3744374, rs56135703, rs117696751, rs2164449, rs150548254, rs7216513, rs148008327, rs9303742, rs148172329, rs148241618, rs12954944, rs8092674, rs146099416, rs8095031, rs2298787, rs17069506, rs17223038, rs968688, rs199667693, rs148965154, rs8102258, rs8112094, rs79579962, rs706764, rs56408444, rs4142943, rs373547599, rs28399491, rs147818341 (same as rs368932391), rs16982743, rs60467951, rs117753855, rs78472358, rs9636153, rs6041749, rs6037509, rs6084431, rs7262903, rs2424943, rs2424944, rs73264111, rs41305787, rs45585744, rs17310782, rs2425007, rs3053708, rs10536288, rs61501849, rs2425014, rs117729108, rs11481073 (same as rs397757541), rs1998233, rs1000411, rs33969356, rs2233884, rs2072974, rs2273086, rs3746619, rs3827103, rs35295928 (same as rs886056870), rs150212573, rs147911723, rs9636833, rs138340460, rs5992854, rs201400555, rs4820658, rs11090413, and rs470101.

The first SNP site combination related to spherical equivalent of the left eye described herein comprises: one or more of rs146160422, rs75054661, rs4959012, rs757262, rs757259, rs145293869, rs34649612, rs7157977, rs563363438, rs2277533, rs7258847, rs78661674, rs5845912, and rs8137790.

The second SNP site combination related to spherical equivalent of the left eye described herein comprises: one or more of rs146160422, rs1051097, rs12129653, rs17553619, rs2275856, rs150943357, rs117712357, rs192688643, rs75054661, rs2292661, rs186309103, rs11556167, rs9790356, rs9790628, rs7717970, rs12519352, rs11133847, rs2304068, rs4959012, rs757262, rs757259, rs929156, rs236444, rs17138865, rs17545405, rs148474510, rs8178175, rs55991828, rs12549018, rs529268660, rs145293869, rs1627784, rs200536689, rs573212, rs4745933, rs3751031, rs12811832, rs11168338, rs2306436, rs3741990, rs34649612, rs7157977, rs17646457, rs563363438, rs2277533, rs2452524, rs4904, rs77707876, rs370627229, rs9929443, rs149149128, rs144944402, rs113276760, rs17682036, rs59176398, rs732109, rs11260004, rs11882197, rs13535, rs2241356, rs57747888, rs397764300_rs58960485, rs16015, rs151309111, rs77561511, rs78422130, rs7258847, rs78661674, rs2183573, rs9981301, rs4624474, rs369859899, rs5845912, rs9628315, rs8138121, and rs8137790.

The third SNP site combination related to spherical equivalent of the left eye described herein comprises: one or more of rs28566954, rs377223831, rs41274480, rs146160422, rs12049046, rs16830693, rs1051097, rs12129487, rs111686948, rs12129653, rs7527057, rs144990398, rs17553619, rs140141416, rs17024525, rs117354680, rs3747626, rs16861256, rs7542316, rs7531786, rs2231241, rs2231240, rs10911390, rs10911391, rs10911392, rs12045762, rs16861394, rs6661715, rs13374714, rs13375853, rs2275856, rs6676149, rs3754234, rs2273584, rs6696123, rs11583387, rs3768066, rs3820553, rs1063128, rs3738521, rs3890478, rs28383065, rs6720415, rs6546511, rs150943357, rs77408734, rs117712357, rs3821036, rs192688643, rs6734083, rs68104327, rs35996697, rs151056400, rs116828761, rs28538922, rs138467334, rs2305613, rs115586700, rs76657333, rs79162067, rs75054661, rs4387990, rs117336796, rs77786095, rs2292661, rs12630803, rs3774765, rs1800766, rs56106326, rs139051035, rs7696197, rs80050773, rs10019009, rs61732380, rs17029087, rs3817083, rs10026790, rs11944752, rs3816873, rs2718684, rs12648093, rs186309103, rs11556167, rs9790356, rs9790628, rs4956981, rs13171103, rs4956987, rs7717970, rs12519352, rs11133847, rs117918201, rs56349002, rs2277042, rs6875303, rs6860528, rs25782, rs117977823, rs11959659, rs2304068, rs192194, rs147518598, rs74403861, rs3734056, rs139635794, rs1611212, rs28400887, rs9261424, rs4959012, rs757262, rs757259, rs929156, rs763046, rs236444, rs73733155, rs2100375, rs1051429, rs17181352, rs8191890, rs61236605, rs142944427, rs3735384, rs17138865, rs17545405, rs140518317, rs2214098, rs148474510, rs555905346, rs6945709, rs10240503, rs2074690, rs374377569, rs200506987, rs6996950, rs145988500, rs6558166, rs149587418, rs8178175, rs55991828, rs117571873, rs77428666, rs12549018, rs529268660, rs1815549, rs139669465, rs145293869, rs1627784, rs139212809, rs200536689, rs77023914, rs573212, rs28369760, rs185006838, rs75361513, rs139337135, rs75911175, rs17148873, rs35535797, rs28434439, rs34630110, rs199902798, rs34296355, rs4745933, rs906220, rs71479686, rs7069048, rs7075840, rs115337638, rs188054898, rs146804890, rs36088476, rs4757987, rs41282940, rs2986407, rs7808, rs3751031, rs201491055, rs145854081, rs202199158, rs117855259, rs117414825, rs75079562, rs1042833, rs17487041, rs12228215, rs12811832, rs11168338, rs1799986, rs2306436, rs17015438, rs45502896, rs34623199, rs140205595, rs11082, rs2305901, rs61941459, rs3741990, rs34649612, rs145055159, rs117478978, rs11551723, rs200011804, rs150666338, rs147350400, rs199627749, rs7157977, rs118004333, rs79020288, rs17646457, rs77215854, rs76611205, rs183718030, rs12915554, rs563363438, rs2277533, rs2452524, rs16963739, rs72738980, rs78309240, rs4904, rs77707876, rs3748380, rs200336355, rs3730278, rs2745136, rs2738893, rs1054645, rs527843401, rs145737959, rs2447927, rs370627229, rs2301770, rs1059676, rs9929443, rs41303763, rs149149128, rs144944402, rs2271047, rs11859725, rs113276760, rs372478569_rs71380707, rs71395334, rs11556634, rs11556635, rs3027234, rs17682036, rs144911043, rs16971620, rs41283411, rs4986763, rs4986764, rs4986765, rs540183826, rs16973424, rs146684394, rs712833, rs73350377, rs6728, rs181969864, rs59176398, rs732108, rs732109, rs76994448, rs75667305, rs28424873, rs28473343, rs3744937, rs368494050, rs146083008, rs11260004, rs11882197, rs2288415, rs10411624, rs13535, rs2217652, rs3826783, rs2230752, rs2241356, rs1978713, rs4804105, rs57747888, rs397764300_rs58960485, rs139817982, rs16015, rs151309111, rs2286663, rs117237657, rs3745764, rs189041504, rs55811458, rs2233156, rs148509022, rs407731, rs406968, rs388706, rs12052105, rs8108435, rs76579307, rs3745403, rs77561511, rs35336528, rs78422130, rs1043673, rs117172918, rs7258847, rs138317120, rs116904482, rs4815467, rs2073194, rs41275624, rs6057110, rs7828, rs1051421, rs6076122, rs78661674, rs6059011, rs148646155, rs5743533, rs114985890, rs80158178, rs201196272, rs8127537, rs2225425, rs2410104, rs1041439, rs2183573, rs1984748, rs9981301, rs4624474, rs73231245, rs885987, rs756661, rs5748471, rs202112368, rs369859899, rs6001008, rs6001009, rs9607524, rs17848337, rs5996087, rs73165153, rs5845912, rs9628315, rs8138121, rs8137790, rs68178377, and rs2232885.

The first SNP site combination related to sphere of the left eye described herein comprises: one or more of rs12549018, rs529268660, rs3741990, rs34649612, rs2452524, rs57747888, rs77561511, rs78422130, rs2073194, rs9981301, rs4624474, rs5845912, and rs8137790.

The second SNP site combination related to sphere of the left eye described herein comprises: one or more of rs377223831, rs146160422, rs12129653, rs16861256, rs7542316, rs2231241, rs2231240, rs10911390, rs10911392, rs12045762, rs16861394, rs6661715, rs150943357, rs192688643, rs76657333, rs79162067, rs4387990, rs1981745, rs17466625, rs139051035, rs61732380, rs17029087, rs3817083, rs10026790, rs2718684, rs186309103, rs7717970, rs12519352, rs11133847, rs2304068, rs3734056, rs139635794, rs4959012, rs757262, rs757259, rs17181352, rs12549018, rs529268660, rs145293869, rs573212, rs117628421, rs139337135, rs522328, rs2228149, rs3751031, rs145854081, rs1042833, rs12228215, rs12811832, rs2305901, rs61941459, rs3741990, rs34649612, rs1060474, rs7157977, rs2277533, rs2452524, rs4904, rs2447927, rs1657074, rs1059676, rs9929443, rs113276760, rs732109, rs11260004, rs11882197, rs10411624, rs13535, rs2217652, rs2241356, rs74351326, rs4804105, rs57747888, rs397764300 (same as rs58960485), rs16015, rs2286663, rs34364867, rs117237657, rs189041504, rs77561511, rs78422130, rs7258847, rs862708, rs4815467, rs2073194, rs6076122, rs78661674, rs2183573, rs9981301, rs4624474, rs9607524, rs5845912, rs9628315, rs8137790, and rs76476219.

The third SNP site combination related to sphere of the left eye described herein comprises: one or more of rs28566954, rs377223831, rs41274480, rs146160422, rs16830693, rs1051097, rs12129487, rs12129653, rs12403630, rs58464768, rs148811681, rs41305862, rs17553619, rs71637410, rs16861256, rs7542316, rs7531786, rs2231241, rs2231240, rs10911390, rs10911391, rs10911392, rs12045762, rs16861394, rs6661715, rs138246957, rs182522436, rs13374714, rs13375853, rs2275856, rs6676149, rs3754234, rs2273584, rs6696123, rs11583387, rs3768066, rs3820553, rs1063128, rs3738521, rs76576389, rs150943357, rs34363861, rs117712357, rs71423514, rs76709099, rs192688643, rs151056400, rs116828761, rs28538922, rs138467334, rs115586700, rs76657333, rs143137431 (same as rs368878844), rs79162067, rs75054661, rs4387990, rs117336796, rs145158569, rs12630803, rs3774765, rs1800766, rs74737650, rs1981745, rs17466625, rs12330397, rs139051035, rs10019009, rs61732380, rs17029087, rs3817083, rs10026790, rs11944752, rs1800591, rs3816873, rs2718684, rs7677415, rs3796936, rs3828506, rs186309103, rs11556167, rs9790356, rs9790628, rs4956981, rs13171103, rs4956987, rs7717970, rs12519352, rs11133847, rs117918201, rs26675, rs1697144, rs2304068, rs147518598, rs57930479, rs140779761, rs74403861, rs3734056, rs139635794, rs10900967, rs1611212, rs28400887, rs9261424, rs4959012, rs757262, rs757259, rs929156, rs3888778, rs75541627, rs117340770, rs9468812, rs60399658, rs189316271, rs186815309, rs11966699, rs73733155, rs3800023, rs2273264, rs6933125, rs17181352, rs8191890, rs142944427, rs1802846, rs1131372, rs3807589, rs200730019, rs17138865, rs17545405, rs775410128, rs555905346, rs6945709, rs2074690, rs200506987, rs3739308, rs3735959, rs145988500, rs6558165, rs6558166, rs149587418, rs55991828, rs151304651, rs77428666, rs141149968, rs12549018, rs529268660, rs7003945, rs200070447, rs1815549, rs139669465, rs145293869, rs1627784, rs573212, rs28369760, rs185006838, rs117628421, rs75361513, rs41415046, rs139337135, rs522328, rs515182, rs482712, rs10760500, rs2228149, rs34630110, rs703075, rs9337951, rs906220, rs906221, rs145472189, rs146884162, rs188054898, rs541533723, rs552775933, rs146804890, rs2280134, rs36088476, rs4757987, rs2986407, rs7808, rs3751031, rs4645933, rs139692587, rs76958954, rs145854081, rs5744247, rs202199158, rs117855259, rs117414825, rs2291075, rs117665966, rs1042833, rs17487041, rs12228215, rs12811832, rs11168338, rs138189784, rs75577568, rs2306436, rs369302829, rs2305901, rs61941459, rs3741990, rs34649612, rs1248046, rs145055159, rs117478978, rs200011804, rs150666338, rs1060474, rs10139379, rs8008670, rs8009789, rs8022395, rs7155123, rs199627749, rs7157977, rs79020288, rs183718030, rs9972231, rs563363438, rs2277533, rs2452524, rs78309240, rs4904, rs77707876, rs1545119, rs3748380, rs200336355, rs2745136, rs60526088, rs2738893, rs1054645, rs2447927, rs1657074, rs370627229, rs2301770, rs1059676, rs9929443, rs55969568, rs71220135, rs16943483, rs11859725, rs113276760, rs5818479, rs71395334, rs78806449, rs1043592, rs17682036, rs144911043, rs16971620, rs540183826, rs16973424, rs146684394, rs80345815, rs732109, rs79712753, rs28424873, rs189184481, rs61735591, rs7251165, rs146083008, rs147472740, rs4134853, rs11260004, rs117622602, rs11882197, rs10411624, rs13535, rs2217652, rs3826783, rs2230752, rs2241356, rs74351326, rs1978713, rs4804105, rs57747888, rs397764300 (same as rs58960485), rs139817982, rs16015, rs151309111, rs2286663, rs34364867, rs117237657, rs189041504, rs1865094, rs2233156, rs2302987, rs148509022, rs188387562, rs407731, rs406968, rs388706, rs12052105, rs3745403, rs34604090, rs77561511, rs35336528, rs78422130, rs7258847, rs862708, rs2361119, rs4801478, rs4815467, rs2073194, rs6076122, rs78661674, rs11546155, rs114985890, rs2236001, rs3989369, rs8127537, rs2225425, rs2410104, rs1041439, rs2183573, rs1984748, rs9981301, rs4624474, rs1051367, rs2073733, rs885985, rs1803545, rs148650930, rs2072012, rs9607524, rs17523588, rs17848337, rs73165153, rs5845912, rs9628315, rs8137790, rs76476219, rs55861809, and rs68178377.

The first SNP site combination related to cylinder of the left eye described herein comprises: one or more of rs26173, rs26098, rs26107, rs26086, rs143580489, rs10484690, and rs117984746.

The second SNP site combination related to cylinder of the left eye described herein comprises: one or more of rs2296474, rs2765015, rs3766165, rs145378993, rs13306137, rs147707514, rs112218972, rs3796381, rs26173, rs26098, rs26107, rs26086, rs987106, rs75126858, rs61734326, rs2278226, rs6928630, rs143580489, rs1204114, rs10484690, rs74699763, rs11574345, rs4925828, rs304494, rs304439, rs304448, rs4763975, rs377238277, rs144650014, rs12438118, rs41303763, rs9797618, rs10420552, rs4802029, rs28512414, rs1975937, rs2075284, rs3095726, rs34785154, rs60704260, rs6084431, rs147911723, rs41279352, rs2427624, and rs117984746.

The third SNP site combination related to cylinder of the left eye described herein comprises: one or more of rs2296474, rs307377, rs2765015, rs3766165, rs145378993, rs61746709, rs114369626, rs4147830, rs540468026, rs80105432, rs45445497, rs13306137, rs191506876, rs147707514, rs4149520, rs4988956, rs4988957, rs10192036, rs10204137, rs4988958, rs10192157, rs10206753, rs3755276, rs112218972, rs3749022, rs76999397, rs35220123, rs80341144, rs3796381, rs117486807, rs147434354, rs76469954, rs26173, rs26098, rs26107, rs26086, rs987106, rs75126858, rs61734326, rs2278226, rs144289000, chr5-140568680, rs7701410, rs117600221, rs150856424, rs3188, rs6928630, rs143580489, rs1204114, rs10484690, rs775410128, rs13225910, rs61733329, rs189212243, rs74699763, rs11574345, rs8178175, rs10092164, rs11539113, rs68008113, rs7462883, rs66881636, rs61747644, rs2721167, rs2620644, rs2250891, rs2250657, rs2721176, rs4925828, rs117569740, rs142643091, rs140966742, rs3758464, rs16935560, rs144762118, rs7899453, rs3740252, rs304494, rs304439, rs304448, rs148238084, rs622082, rs546382, rs189385634, rs4763975, rs2232566, rs7132546, rs5742612, rs35767, rs2861550, rs377238277, rs144650014, rs35405829, rs11569599, rs10130914, rs140879339, rs12438118, rs182015467, rs41303763, rs142805492, rs8055740, rs75743672, rs75277269, rs76896732, rs2164449, rs2230601, rs9303742, rs79577494, rs9797618, rs10420552, rs4802029, rs28512414, rs1975937, rs958305, rs2075284, rs3095726, rs34785154, rs60704260, rs2034891, rs117753855, rs78472358, rs8099961, rs58634535, rs6084431, rs147911723, rs41279352, rs2427624, rs117984746, rs470101, and rs2267163.

The fourth SNP site combination related to cylinder of the left eye described herein comprises: one or more of rs6690013, rs609805, rs621668, rs2274264, rs2274263, rs2296474, rs10907179, rs307349, rs307355, rs35744813, rs307377, rs307371, rs2296471, rs2765015, rs34091023 (same as rs371194568), rs1171, rs3766165, rs145378993, rs115226069, rs61746709, rs2274329, rs2274330, rs3765967, rs148750290, rs114369626, rs35533335, rs74622002, rs145619910, rs141216171, rs146227933, rs145717494, rs4147830, rs3215952, rs2229783, rs17127203, rs1676486, rs1763347, rs368345074, rs7556386, rs540468026, rs374309842, rs942963, rs77181873, rs80105432, rs33941127, rs45445497, rs12026637, rs144649977, rs78780269, rs3813972, rs60555365, rs335524, rs4149230, rs2292559, rs2292561, rs41310561, rs147519768, rs13306137, rs1800672, rs2296801, rs77082151, rs76255469, rs12135078, rs112212885, rs62121538, rs191506876, rs147707514, rs74867758, rs4665385, rs3792253, rs1126497, rs4149520, rs3771175, rs4988956, rs4988957, rs10192036, rs10204137, rs4988958, rs10192157, rs10206753, rs3755276, rs112218972, rs116177854, rs2123465, rs2116664, rs1469651, rs1469650, rs1053394, rs77698806, rs1434087, rs3749022, rs145346406, rs6734083, rs11676792, rs53576, rs76999397, rs2075310, rs35993975, rs33917318, rs17032276, rs17032278, rs17032283, rs35220123, rs9879080, rs7649243, rs34608006, rs6805869, rs9811771, rs2272125, rs2272124, rs7626117, rs7647987, rs6796538, rs35229877, rs11919713, rs1969154, rs1985428, rs3732675, rs80341144, rs181735010, rs3796381, rs76407344, rs11719458, rs2292661, rs79845192, rs74682201, rs77220512, rs35070271, rs79144888, rs76371354, rs78233713, rs117486807, rs147434354, rs6815254, rs3756247, rs1457976435, rs191674075, rs3749584, rs10517195, rs78205702, rs76469954, rs26173, rs26098, rs26107, rs26086, rs987106, rs62387135, rs79420400, rs75126858, rs61734326, rs2278226, rs2228112, rs42688, rs32210, rs6886922, rs118179072, rs144289000, chr5-140568680, rs182889312, rs7701410, rs117600221, rs142883877, rs150856424, rs3734391, rs140750807, rs138075877, rs2274347, rs3188, rs62622418, rs74609186, rs9396251, rs6928630, rs143580489, rs1204114, rs10484690, rs370140172, rs1048886, rs78873069, rs73333875, rs200555224, rs80190153, rs201920957, rs150559408, rs775410128, rs189382707, rs79848686, rs17171113, rs7791608, rs17173702, rs17173703, rs13225910, rs61733329, rs189212243, rs74699763, rs11550509, rs10866818, rs11574345, rs8178175, rs10092164, rs11539113, rs34596274, rs68008113, rs7462883, rs66881636, rs7812496, rs3739287, rs61747644, rs7840439, rs2721167, rs2620644, rs2250891, rs2250657, rs2721176, rs4925828, rs2721190, rs2721189, rs2721172, rs2281747, rs10961672, rs140349388_rs3833713, rs7023474, rs80235406, rs4879781, rs112858544, rs1164863576, rs6560142, rs879369, rs117569740, rs4838184, rs371047734, rs522328, rs515182, rs482712, rs199901947, rs12684476, rs7043416, rs113809617, rs7079747, rs142643091, rs74704054, rs140966742, rs3758464, rs16935560, rs144762118, rs7899453, rs3740252, rs2762528, rs304494, rs304439, rs304448, rs2254619, rs11017735, rs2298192, rs193087745, rs756924, rs2074022, rs739502, rs12577654, rs148238084, rs111885243, rs57477011, rs2074233, rs2067477, rs77749149, rs145270731, rs622082, rs546382, rs189385634, rs2852365, rs1057992, rs642573, rs3018301, rs12272086, rs5103, rs5069, rs377232839, rs201872688, rs4763975, rs150028933, rs2232566, rs7132546, rs2304821, rs148804891, rs5742612, rs35767, rs10778292, rs11112201, rs10847164, rs117739120, rs185257411, rs145509540, rs2861550, rs2282407, rs377238277, rs144650014, rs35405829, rs145005726, rs45585336, rs186889719, rs11569599, rs11570779, rs76846944, rs698028, rs10130914, rs1808975, rs140879339, rs9919918, rs11853872, rs25397, rs3075142_rs397774483, rs12904657, rs12438118, rs7170185, rs17273206, rs3815003, rs2227933, rs74839044, rs34855511, rs577645079, rs76468382, rs117474153, rs367960426, rs182015467, rs41303763, rs187940556, rs13337155, rs138428580, rs1055708, rs62054627, rs1048149, rs62052673, rs142805492, rs8049480, rs758803114, rs8055740, rs138169571, rs7222126, rs11657498, rs200205820, rs4796407, rs2302763, rs138625739, rs201373267, rs2302160, rs34747500, rs56135703, rs28691051, rs200996148, rs75743672, rs729782, rs117696751, rs75277269, rs76896732, rs2164449, rs2230601, rs9303742, rs35114461, rs17223038, rs148965154, rs79577494, rs79579962, rs1054533, rs9797618, rs10420552, rs4802029, rs28512414, rs1975937, rs958305, rs2075284, rs3095726, rs34785154, rs60704260, rs112805435, rs188724242, rs2034891, rs117753855, rs112910446, rs8100225, rs1657535, rs78472358, rs8099961, rs142900000, rs8958, rs58634535, rs6084431, rs2280091, rs2280092, rs3746642, rs235768, rs235769, rs221984, rs2424943, rs2424944, rs73264111, rs41305787, rs45585744, rs2295096, rs2071971, rs117358971, rs112959405, rs1124178, rs3746619, rs3827103, rs35295928_rs886056870, rs150212573, rs147911723, rs41279352, rs2427624, rs139729932, rs117984746, rs2276254, rs141212516 (same as rs377432980 or rs386365935 or rs386394882), rs7283989, rs11701124, rs8131313, rs35796750, rs143527452, rs45604134, rs1052756, rs1052773, rs2240111, rs165815, rs2073747, rs5762430, rs470101, rs2267163, rs5997714, rs117291362, and rs11553143.

### Kit

In another aspect, the present invention further provides a kit for detecting a vision condition, comprising a reagent for detecting any one of the SNP site combinations described above.

Optionally, the reagent for detecting the SNP site combination includes, but is not limited to, reagents used in the following methods for detecting SNPs: a TaqMan probe method, a sequencing method, a chip method, time of flight mass spectrometer (MALDI-TOFMS) detection, a restriction fragment-length polymorphism (PCR-RFLP) method, a single strand conformation polymorphism (PCR-SSCP) method, allele-specific PCR (AS-PCR), a SNaPshot method, a SNPlex method, denaturing high performance liquid chromatography (DHPLC), and denaturing gradient gel electrophoresis (DGGE). Those skilled in the art can select any one or more of the methods for detecting SNP sites as long as the detection of the SNP sites can be achieved.

Optionally, the reagent for detecting the SNP site combination may further include a reagent for amplifying the SNP site combination described above.

Optionally, the kit may further comprise common reagents required for corresponding PCR techniques, such as dNTPs, MgCl₂, double distilled water, and fluorescent probes, which are well known to those skilled in the art, and may additionally comprise a standard and a control (such as a genotype standard and a blank control).

Optionally, the reagent includes, but is not limited to, a primer, a probe, a chip, and the like.

Optionally, the kit further comprises a reagent and/or an instrument for processing a sample.

The term “sample” as used herein refers to any sample containing nucleic acid (particularly DNA) from or derived from a human patient, for example, a sample extracted from oral mucosal cells, oral exfoliated cells, saliva, blood, dried blood spots, and the like of the patient, as can essentially any target tissue, cell, and body fluid containing the appropriate nucleic acid. In one embodiment, the sample is an oral swab.

### Detection System

In another aspect, the present invention provides a vision condition detection system, comprising a computing device for determining a vision condition of a subject using a detection result for the SNP site combination described above.

Optionally, the subject described herein is a human (adolescent) of 6-18 years old. More optionally, the subject described herein is a human (adolescent) of 6-18 years old in Asia.

Optionally, the diagnostic system may further include a detection device for the SNP sites, e.g., sequencer, etc.

Optionally, the detection system further includes a data collection unit for collecting data, including SNP data, from a subject.

Optionally, the detection system further includes a model creation unit for creating a myopia diagnostic model according to the construction method described above.

Optionally, the detection system further includes a model optimization unit for optimizing the myopia diagnostic model.

It should be understood that the term “system”, “device”, or “unit” as used herein is a method for distinguishing different components, elements, parts, portions, or assemblies at different levels. However, these words may be substituted by other expressions if they accomplish the same purpose.

### Method for Constructing Model

In another aspect, the present invention provides a method for constructing a vision condition detection model using the above SNP site combinations.

Optionally, the method is a logistic regression method. As used herein, “logistic regression” refers to a method for predicting the likelihood of an event occurring by fitting data to a logistic curve. Those skilled in the art will understand how to use this method in the context of the present invention.

Optionally, the present invention further comprises a step of performing 10-fold cross-validation. The “10-fold cross-validation” specifically includes selecting 10 random numbers from 0 to 100 as random seeds by using a sklearn.model_selection function, dividing samples into 10 groups of training and validation sets, performing a 10-fold cross-validation, and taking an average of 10 results as a model result.

### Model

In another aspect, the present invention further provides a model for detecting a vision condition, which determines a current vision condition of a subject according to a detection result for the SNP site combination described above.

Optionally, the model is constructed by the method for constructing the vision condition detection model described above.

Optionally, the model may be a formula, a nomogram, or other means of facilitating manipulation by the subject; according to the model, the current vision condition of the subject can be directly obtained by calculation or the possibility of developing a vision problem in the future can be predicted.

### Method

In another aspect, the present invention provides a method for detecting a vision condition or predicting the possibility of developing a vision problem, comprising a step of determining a vision condition of a subject according to a detection result for the SNP site combination according to the present invention.

Optionally, the method may comprise the following steps:
1) collecting a sample from a subject;
2) performing SNP detection on the sample; and
3) determining a vision condition of the subject or predicting the possibility of developing a vision problem according to a result for the detection of 2).

In another aspect, the present invention provides a method for preventing or treating a vision problem, comprising:
step 1, detecting biological samples from subjects to obtain genotypes of any one of the SNP site combinations described herein;
step 2, inputting results obtained by the detection in step 1 into a myopia model for processing, and classifying the subjects according to the genotypes of the SNP site combination, and
step 3, administering corrective lenses or administering a pharmaceutically effective amount of a therapeutic agent to those susceptible to a vision problem selected by the classification in step 2.

The corrective lenses of the present invention include orthokeratology lenses (Ortho-K) or blur-inducing lenses (e.g., as described in international patent application publication No. WO 2010/075319).

Optionally, the corrective lenses include spectacles and contact lenses.

Optionally, the myopia model is constructed by logistic regression.

Optionally, the myopia model is determined by the following steps: traversing all values of a threshold, separately establishing models and calculating AUCs, and taking a model with a maximum AUC as a final myopia model.

Optionally, the sample comprises a sample taken directly from the subject, or, a sample subjected to treatment (e.g., purification, extraction of DNA, or amplification of SNP sites); optionally, the sample is an oral swab (oral mucosa sample).

Optionally, the determination may be made manually, automatically, or in combination to perform or complete the selected task; the results may be calculated manually according to the detection results or automatically by inputting the detection results into the detection system or model.

In another aspect, the present invention provides a predictive analysis method for myopia, having a schematic flow diagram as shown in FIG. 1.

### Apparatus

In another aspect, the present invention provides a predictive analysis apparatus for myopia or use thereof in determining a vision condition of a subject, the apparatus comprising: a memory and a processor, wherein,
the memory is configured to store program instructions;
the processor is configured to invoke the program instructions, which, when executed, perform the predictive analysis method for myopia or the method for detecting a vision condition or predicting the possibility of developing a vision problem described above.

### Computer-Readable Storage Medium

In another aspect, the present invention provides a computer-readable storage medium or use thereof in determining a vision condition of a subject, the computer-readable storage medium having a computer program stored thereon, which, when executed by a processor, implements the predictive analysis method for myopia or the method for detecting a vision condition or predicting the possibility of developing a vision problem described above.

As will be understood by those skilled in the art, the present invention may be embodied as an apparatus, a method, or a computer program product. Accordingly, the present disclosure may be embodied in the following form: complete hardware, complete software (including firmware, resident software, micro-code, etc.), or a combination of hardware and software.

### Use

In another aspect, the present invention provides use of any one of the following in detecting myopia or high myopia or predicting the possibility of developing myopia or high myopia:
1) the SNP site combination related to myopia or high myopia described above, 2) a reagent for detecting 1), 3) a kit consisting of the reagent for detecting 1), 4) a detection system comprising a computing device for determining a vision condition of a subject according to a detection result for 1), and 5) a model for detecting myopia or high myopia constructed by using 1).

In another aspect, the present invention provides use of any one of the following in detecting spherical equivalent difference between the two eyes or predicting the possibility of developing myopia or high myopia:
1) the SNP site combination related to spherical equivalent difference between the two eyes, 2) a reagent for detecting 1), 3) a kit consisting of the reagent for detecting 1), 4) a detection system comprising a computing device for determining a vision condition of a subject according to a detection result for 1), and 5) a model for detecting spherical equivalent difference between the two eyes constructed by using 1).

In another aspect, the present invention provides use of any one of the following in detecting spherical equivalent of the worst eye or predicting the possibility of developing myopia or high myopia:
1) the SNP site combination related to spherical equivalent of the worst eye described above, 2) a reagent for detecting 1), 3) a kit consisting of the reagent for detecting 1), 4) a detection system comprising a computing device for determining a vision condition of a subject according to a detection result for 1), and 5) a model for detecting spherical equivalent of the worst eye constructed by using 1).

In another aspect, the present invention provides use of any one of the following in detecting spherical equivalent of the right eye or predicting the possibility of developing myopia or high myopia:
1) rs2547319 or the SNP site combination related to spherical equivalent of the right eye described above, 2) a reagent for detecting 1), 3) a kit consisting of the reagent for detecting 1), 4) a detection system comprising a computing device for determining a vision condition of a subject according to a detection result for 1), and 5) a model for detecting spherical equivalent of the right eye constructed by using 1).

In another aspect, the present invention provides use of any one of the following in detecting sphere of the right eye or predicting the possibility of developing myopia or high myopia:
1) rs143477571 or the SNP site combination related to sphere of the right eye described above, 2) a reagent for detecting 1), 3) a kit consisting of the reagent for detecting 1), 4) a detection system comprising a computing device for determining a vision condition of a subject according to a detection result for 1), and 5) a model for detecting sphere of the right eye constructed by using 1).

In another aspect, the present invention provides use of any one of the following in detecting cylinder of the right eye or predicting the possibility of developing myopia or high myopia:
1) the SNP site combination related to cylinder of the right eye described above, 2) a reagent for detecting 1), 3) a kit consisting of the reagent for detecting 1), 4) a detection system comprising a computing device for determining a vision condition of a subject according to a detection result for 1), and 5) a model for detecting cylinder of the right eye constructed by using 1).

In another aspect, the present invention provides use of any one of the following in detecting spherical equivalent of the left eye or predicting the possibility of developing myopia or high myopia:
1) the SNP site combination related to spherical equivalent of the left eye described above, 2) a reagent for detecting 1), 3) a kit consisting of the reagent for detecting 1), 4) a detection system comprising a computing device for determining a vision condition of a subject according to a detection result for 1), and 5) a model for detecting spherical equivalent of the left eye constructed by using 1).

In another aspect, the present invention provides use of any one of the following in detecting sphere of the left eye or predicting the possibility of developing myopia or high myopia:
1) the SNP site combination related to sphere of the left eye described above, 2) a reagent for detecting 1), 3) a kit consisting of the reagent for detecting 1), 4) a detection system comprising a computing device for determining a vision condition of a subject according to a detection result for 1), and 5) a model for detecting sphere of the left eye constructed by using 1).

In another aspect, the present invention provides use of any one of the following in detecting cylinder of the left eye or predicting the possibility of developing myopia or high myopia:
1) the SNP site combination related to cylinder of the left eye described above, 2) a reagent for detecting 1), 3) a kit consisting of the reagent for detecting 1), 4) a detection system comprising a computing device for determining a vision condition of a subject according to a detection result for 1), and 5) a model for detecting cylinder of the left eye constructed by using 1).

### BRIEF DESCRIPTION OF THE DRAWINGS

FIG. 1 is a schematic flow diagram of a predictive analysis method for myopia according to an example of the present invention.

FIG. 2 is a ROC curve of the SNP site combinations related to myopia or high myopia according to the present invention.

FIG. 3 shows change curves of AUC values of the SNP combinations selected by screening of the present invention when determining the vision condition at different thresholds, wherein A: spherical equivalent difference between the two eyes (diff se), B: spherical equivalent of the worst eye (worst se), C: spherical equivalent of the right eye (od_se), D: spherical equivalent of the left eye (os_se), E: cylinder of the right eye (rcyl), F: cylinder of the left eye (lcyl), G: sphere of the right eye (rsph), and H: sphere of the left eye (lsph).

### DETAILED DESCRIPTION

The present invention is further described with reference to the following examples. The following description is only for the purpose of illustrating preferred examples of the present invention, and not to be limiting of the present invention in any way. Any person skilled in the art may take advantage of the technical content disclosed above and change it to equivalent examples with equivalent variations. Any simple modifications or equivalent changes made to the following examples according to the spirit of the present invention, without departing from the content of the solutions of the present invention, fall within the scope of the present invention.

### General Method: Sample Collection and Sequencing

### Inclusion Criteria for Samples:

(1) The spherical equivalent of the worst eye of less than or equal to −6.00D;
(2) age: 6-18 years old;
(3) no other ocular diseases, congenital genetic diseases, other systemic diseases, and physical abnormalities;
(4) no history of ocular surgery;
(5) Chinese people; and
(6) voluntary participation and signed informed consent.

In the present invention, sample information of the myopia screening and intervention project of primary and secondary school students in Wenzhou city, which was carried out by the Eye Hospital of Wenzhou Medical University in cooperation with the Wenzhou Municipal Bureau of Government Services in 2019-2020, was collated, 10,348 (9852-8961) samples meeting the inclusion criteria were gathered for whole exome sequencing, the sphere (Sph) and the cylinder (Cyl) of the two eyes of the samples were recorded, and the spherical equivalent (SE=Sph+0.5 Cyl) of the two eyes of the samples was calculated. Meanwhile, the 10,348 cases of patients with high myopia were subjected to whole exome sequencing to obtain SNPs of the whole exome, and SNPs related to traits were obtained through exome-wide association analysis.

### Specific Experimental Procedures:

In the examples, oral mucosa samples meeting the standard were collected by a standard operating procedure (SOP), patients meeting the inclusion criteria were systematically collected and followed up, whole exons were sequenced using an Illumina Novaseq6000 sequencing system to detect SNPs in the whole exon range, SNP markers and combinations thereof related to the spherical equivalent (SE) of a high myopia population were explored, and a model for predicting the progress risk of spherical equivalent was constructed.
1. Oral mucosa swab collection: Food and water intake were stopped half an hour before sample collection to ensure that the samples were not contaminated. The collected oral mucosa swabs were each put into a sterilized 1.5 mL EP tube, and after filling in the delivery registration form, the samples were transported to the company at normal temperature and handed to a professional for extracting the genomic DNA from the oral mucosa swabs.
2. DNA extraction: Genomic DNA was extracted from the oral mucosal swabs using the TIANamp swab DNA kit, specifically as follows:
(1) The oral mucosa swabs were each transferred into a 2 mL centrifuge tube, the swab portion was cut from the swab rod with scissors, and 400 μL of buffer GA was added.
(2) 20 μL of proteinase K solution was added, and the tube was vortexed for 10 s for uniform mixing and left to stand at 56° C. for 60 min, during which vortex was performed every 15 min for several times.
(3) 400 μL of buffer GB was added, and the tube was turned upside down for uniform mixing and left to stand at 70° C. for 10 min. At this point, the solution should become clear. The tube was centrifuged briefly to remove the liquid droplets on the inner wall of the tube cap, and then squeezed to remove the swab, and as much lysate as possible was transferred to a new centrifuge tube.
(4) 200 μL of absolute ethanol was added, and the tube was turned upside down for uniform mixing, and centrifuged briefly to remove liquid droplets on the inner wall of the tube cap.
(5) The solution and flocculent precipitate obtained in the previous step were added to an adsorption column CR2 (the adsorption column CR2 was placed into a collection tube), the tube was centrifuged at 12,000 rpm (about 13,400×g) for 30 s, the waste liquid in the collection tube was discarded, and the adsorption column CR2 was placed back into the collection tube.
(6) 500 μL of buffer GB (please confirm that absolute ethanol has been added before use) was added to the adsorption column CR2, the tube was centrifuged at 12,000 rpm (about 13,400×g) for 30 s, the waste liquid in the collection tube was discarded, and the adsorption column CR2 was placed back into the collection tube.
(7) 700 μL of rinsing solution PW (please confirm that absolute ethanol has been added before use) was added to the adsorption column CR2, the tube was centrifuged at 12,000 rpm (about 13,400×g) for 30 s, the waste liquid in the collection tube was discarded, and the adsorption column CR2 was placed back into the collection tube.
(8) Step 7 was repeated.
(9) The tube was centrifuged at 12,000 rpm (13,400×g) for 2 min, the waste liquid was discarded, and the adsorption column CR2 was left to stand at room temperature for several minutes to completely dry the adsorption material to remove residual rinsing solution therein.
(10) The adsorption column CR2 was transferred into a clean centrifuge tube, 20-50 μL of elution buffer TB was added dropwise from above to the middle of the adsorption film, and the tube was left to stand at room temperature for 2-5 min and centrifuged at 12,000 rpm (13,400×g) for 2 min.
(11) The concentration and purity of DNA fragments were determined by agarose gel electrophoresis and UV spectrophotometer. DNA had a significant absorption peak at OD260, and the OD260 value of 1 was equivalent to 50 μg/mL double-stranded DNA or 40 μg/mL single-stranded DNA. The purity (OD 260/280) was in the range of 1.7-1.9. 0.5-3.5 μg of DNA can generally be obtained from a single sample.
3. Pre-library construction: 50 ng of genomic DNA was broken into small fragments of about 200 bp by an enzymatic method, then terminal repair and 3′ terminus A-tailing operations were performed, the DNA fragments were ligated with sequencing adapters containing barcode sequences, the fragments of about 320 bp were selected and recovered, and a pre-library was obtained after PCR amplification.
4. Liquid-phase hybrid capture operation: The pre-library was subjected to a liquid-phase hybrid capture operation with reference to the standard procedure of xGen Exome Research Panel V1.0 (Integrated DNA Technologies, San Diego, USA) of IDT.
5. Exon library acquisition: The captured products, after being eluted and recovered, were subjected to PCR amplification and purification to obtain an exon library. The library was quantified by qPCR and the band size was measured using Agilent 2100.
6. Illumina Novaseq6000 sequencing: 150PE sequencing was performed on the exon library by using an Illumina Novaseq6000 sequencing system, and base calling was performed on the original image by using CASAVA v1.82 software to produce original sequencing data.
7. Alignment to human reference genome: The sequenced fragments were aligned to the human reference genome (UCSC hg19) using the Burrows-Wheeler Aligner (BWA) tool and PCR repeats were removed using Picard v1.57. Variant detection was performed by adopting software GATK, and the sequencing depth, the coverage depth, the homogeneity, and the like were statistically analyzed.
8. Data quality control: For the samples: the samples with insufficient oral swab sampling amount, with a detection rate of less than 90%, with mean coverage of less than 10, with mean probability of genotypic presence of less than 65%, with mean genotypic heterozygosity deviation of ±4 standard deviations (SD), with chromosomal anomalies, with sex anomalies, with consanguineous relationships, and of the non-East Asian population were deleted. For SNPs: The detection results for whole exome that did not pass VQSR quality control would be SNPs with a genotype detection rate of less than 90%, a Hardy Weinberg P-value of less than 1e-06, and a variant allele count (AC) of 0.
9. Statistical analysis method: A study on quantitative traits of the whole exome of high myopia was performed through Bayesian tests in SNPtest, and potential SNPs and genes significantly related to the clinical state of high myopia were identified. The Bayesian tests of quantitative traits were performed by using a conjugate prior formula of a linear model of the expected genotype (-method expected). For an additive model, the formula is:

$${y}_{i} = β {G}_{i} + {e}_{i} , {e}_{i} ∼ N ( 0 , {σ}^{2} ) .$$

yᵢ denotes a residual phenotype of the ith individual. The residual phenotype is calculated by subtracting one baseline term and estimating any specified covariates. Gi denotes an additive code for the expected genotype of the ith individual, wherein wild homozygous genotype=“0”, heterozygous genotype=“1”, and mutant homozygous genotype=“2”.

σ2 denotes the error variance of the model. σ²˜IG(a,b), β˜N(m_{β},V_{β}σ²), and by default, a=3, b=2, m_{β}=0,V_{β}=0.02. Age and sex are used as covariates for correction.

### Example 1: Schematic Flow of Predictive Analysis Method for Myopia

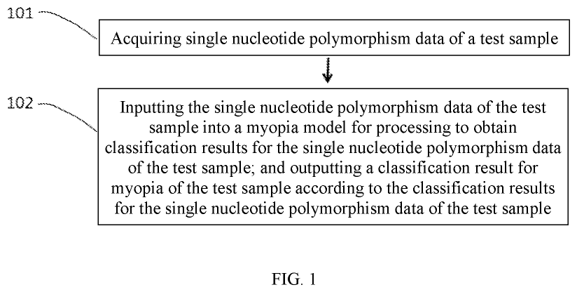

FIG. 1 is a schematic flow diagram of a predictive analysis method for myopia according to an example of the present invention. Specifically, the method comprises the following steps:

101: acquiring single nucleotide polymorphism data of a test sample.

In one example, the single nucleotide polymorphism data is single nucleotide polymorphism data subjected to data quality control. The quality control process comprises:
acquiring original sequencing data, aligning the original sequencing data to a human reference genome, and reserving unique non-repetitive matching data with the human reference genome;
deleting single nucleotide polymorphism data which do not meet the processing standard in the unique non-repetitive matching data to obtain single nucleotide polymorphism data subjected to data quality control; and
selecting target single nucleotide polymorphism data by screening from the single nucleotide polymorphism data subjected to data quality control.

Optionally, for the selected related SNPs by screening, the detection was performed in additional samples to verify the repeatability and stability of the application in clinical diagnosis.

102: inputting the single nucleotide polymorphism data of the test sample into a myopia model for processing to obtain classification results for the single nucleotide polymorphism data of the test sample; and outputting a classification result for myopia of the test sample according to the classification results for the single nucleotide polymorphism data of the test sample.

The myopia model is determined in a mode comprising:
acquiring single nucleotide polymorphism data of a sample from a patient with myopia;
selecting target single nucleotide polymorphism data by screening from the single nucleotide polymorphism data as characteristic data, wherein the target single nucleotide polymorphism data are single nucleotide polymorphism data of genome-wide association with a quantitative trait of myopia; and
performing characteristic selection on the characteristic data by adopting a machine learning method to obtain selected characteristics, and constructing a myopia model by using the selected characteristics to obtain a constructed myopia model.

In one example, the method or step of selecting target single nucleotide polymorphism data by screening from the single nucleotide polymorphism data as characteristic data comprises the following three steps:

The single nucleotide polymorphism data is screened by using an SNPTEST method to obtain initial target single nucleotide polymorphism data.

In one example, the genome-wide association of a total of 89,095 common variants in 8961 high myopia samples with myopia or high myopia was obtained by an SNPTEST method, and then snps were screened by a threshold of −6.0D for modeling (myopia or high myopia model): log 10(BF)>3, resulting in a total of 16 snps, with an AUC of 0.739.

In one example, the genome-wide association of a total of 89,095 common variants in 8961 high myopia samples with a quantitative trait: spherical equivalent difference between the two eyes was obtained by an SNPTEST method, and then snps were screened by 4 thresholds for modeling: log 10(BF)>3; log 10(BF)>2; log 10(BF)>1.5; and log 10(BF)>1, resulting in a total of 341 snps. The submodel of spherical equivalent difference between the two eyes includes one or more of the following submodels: submodel I, submodel II, submodel III, and submodel IV.

Submodel I (screening criteria: log 10(BF)>3, a first SNP site combination related to spherical equivalent difference between the two eyes) has a first threshold of 5.625, an AUC of 0.66, and a number of SNPs of 5.

Submodel II (screening criteria: log 10(BF)>2, a second SNP site combination related to spherical equivalent difference between the two eyes) has a first threshold of 5.375, an AUC of 0.74, and a number of SNPs of 34.

Submodel III (screening criteria: log 10(BF)>1.5, a third SNP site combination related to spherical equivalent difference between the two eyes) has a first threshold of 5.5, an AUC of 0.80, and a number of SNPs of 110.

Submodel IV (screening criteria: log 10(BF)>1, a fourth SNP site combination related to spherical equivalent difference between the two eyes) has a first threshold of 5.625, an AUC of 0.88, and a number of SNPs of 341.

That is, when 5 SNP sites obtained by screening with log bf>3 as criteria are used as markers, whether the spherical equivalent difference between the two eyes of a subject is above 5.625 can be effectively determined; when 34 SNP sites obtained by screening with log bf>2 as criteria are used as markers, whether the spherical equivalent difference between the two eyes of a subject is above 5.375 can be effectively determined; when 110 SNP sites obtained by screening with log bf>1.5 as criteria are used as markers, whether the spherical equivalent difference between the two eyes of a subject is above 5.5 can be effectively determined; when 341 SNP sites obtained by screening with log bf>1 as criteria are used as markers, whether the spherical equivalent difference between the two eyes of a subject is above 5.625 can be effectively determined.

In one example, the genome-wide association of a total of 89,095 common variants in 8961 high myopia samples with a quantitative trait: spherical equivalent of the worst eye was obtained by an SNPTEST method, and then snps were screened by 4 thresholds for modeling: log 10(BF)>3; log 10(BF)>2; log 10(BF)>1.5; and log 10(BF)>1, resulting in a total of 317 snps. The submodel of spherical equivalent of the right eye includes one or more of the following submodels: submodel I, submodel II, submodel III, and submodel IV.

Submodel I (screening criteria: log 10(BF)>3, a first SNP site combination related to spherical equivalent of the worst eye) has a first threshold of −11.375, an AUC of 0.59, and a number of SNPs of 3.

Submodel II (screening criteria: log 10(BF)>2, a second SNP site combination related to spherical equivalent of the worst eye) has a first threshold of −11.875, an AUC of 0.66, and a number of SNPs of 22.

Submodel III (screening criteria: log 10(BF)>1.5, a third SNP site combination related to spherical equivalent of the worst eye) has a first threshold of −11.5, an AUC of 0.77, and a number of SNPs of 90.

Submodel IV (screening criteria: log 10(BF)>1, a fourth SNP site combination related to spherical equivalent of the worst eye) has a first threshold of −11.5, an AUC of 0.79, and a number of SNPs of 317.

In one example, the genome-wide association of a total of 89,095 common variants in 8961 high myopia samples with a quantitative trait: spherical equivalent of the right eye was obtained by an SNPTEST method, and then snps were screened by 4 thresholds for modeling: log 10(BF)>3; log 10(BF)>2; log 10(BF)>1.5; and log 10(BF)>1, resulting in a total of 342 snps. The submodel of spherical equivalent of the right eye includes one or more of the following submodels: submodel I, submodel II, submodel III, and submodel IV.

Submodel I (screening criteria: log 10(BF)>3, rs2547319) has a first threshold of −11.125, an AUC of 0.59, and a number of SNPs of 1.

Submodel II (screening criteria: log 10(BF)>2, a first SNP site combination related to spherical equivalent of the right eye) has a first threshold of −11.125, an AUC of 0.67, and a number of SNPs of 26.

Submodel III (screening criteria: log 10(BF)>1.5, a second SNP site combination related to spherical equivalent of the right eye) has a first threshold of −11.25, an AUC of 0.77, and a number of SNPs of 102.

Submodel IV (screening criteria: log 10(BF)>1, a third SNP site combination related to spherical equivalent of the right eye) has a first threshold of −11.125, an AUC of 0.80, and a number of SNPs of 342.

In one example, the genome-wide association of a total of 89,095 common variants in 8961 high myopia samples with a quantitative trait: sphere of the right eye was obtained by an SNPTEST method, and then snps were screened by 4 thresholds for modeling: log 10(BF)>3; log 10(BF)>2; log 10(BF)>1.5; and log 10(BF)>1, resulting in a total of 367 snps. The submodel of sphere of the right eye includes one or more of the following submodels: submodel I, submodel II, submodel III, and submodel IV.

Submodel I (screening criteria: log 10(BF)>3, rs143477571) has a first threshold of −8.75, an AUC of 0.54, and a number of SNPs of 1.

Submodel II (screening criteria: log 10(BF)>2, a first SNP site combination related to sphere of the right eye) has a first threshold of −10, an AUC of 0.68, and a number of SNPs of 17.

Submodel III (screening criteria: log 10(BF)>1.5, a second SNP site combination related to sphere of the right eye) has a first threshold of −9.75, an AUC of 0.72, and a number of SNPs of 114.

Submodel IV (screening criteria: log 10(BF)>1, a third SNP site combination related to sphere of the right eye) has a first threshold of −9.75, an AUC of 0.77, and a number of SNPs of 367.

In one example, the genome-wide association of a total of 89,095 common variants in 8961 high myopia samples with a quantitative trait: cylinder of the right eye was obtained by an SNPTEST method, and then snps were screened by 4 thresholds for modeling: log 10(BF)>3; log 10(BF)>2; log 10(BF)>1.5; and log 10(BF)>1, resulting in a total of 423 snps. The submodel of sphere of the right eye includes one or more of the following submodels: submodel I, submodel II, submodel III, and submodel IV.

Submodel I (screening criteria: log 10(BF)>3, a first SNP site combination related to cylinder of the right eye) has a first threshold of −2.5, an AUC of 0.56, and a number of SNPs of 4.

Submodel II (screening criteria: log 10(BF)>2, a second SNP site combination related to cylinder of the right eye) has a first threshold of −3.75, an AUC of 0.68, and a number of SNPs of 31.

Submodel III (screening criteria: log 10(BF)>1.5, a third SNP site combination related to cylinder of the right eye) has a first threshold of −3.75, an AUC of 0.73, and a number of SNPs of 98.

Submodel IV (screening criteria: log 10(BF)>1, a fourth SNP site combination related to cylinder of the right eye) has a first threshold of −4.25, an AUC of 0.77, and a number of SNPs of 423.

In one example, the genome-wide association of a total of 89,095 common variants in 8961 high myopia samples with a quantitative trait: spherical equivalent of the left eye was obtained by an SNPTEST method, and then snps were screened by 3 thresholds for modeling: log 10(BF)>2; log 10(BF)>1.5; and log 10(BF)>1, resulting in a total of 347 snps. The submodel of spherical equivalent of the left eye includes one or more of the following submodels: submodel I, submodel II, and submodel III.

Submodel I (screening criteria: log 10(BF)>2, a first SNP site combination related to spherical equivalent of the left eye) has a first threshold of −11, an AUC of 0.63, and a number of SNPs of 14.

Submodel II (screening criteria: log 10(BF)>1.5, a second SNP site combination related to spherical equivalent of the left eye) has a first threshold of −10.75, an AUC of 0.68, and a number of SNPs of 76.

Submodel III (screening criteria: log 10(BF)>1, a third SNP site combination related to spherical equivalent of the left eye) has a first threshold of −10.875, an AUC of 0.76, and a number of SNPs of 347.

In one example, the genome-wide association of a total of 89,095 common variants in 8961 high myopia samples with a quantitative trait: sphere of the left eye was obtained by an SNPTEST method, and then snps were screened by 3 thresholds for modeling: log 10(BF)>2; log 10(BF)>1.5; and log 10(BF)>1, resulting in a total of 347 snps. The submodel of sphere of the left eye includes one or more of the following submodels: submodel I, submodel II, and submodel III.

Submodel I (screening criteria: log 10(BF)>2, a first SNP site combination related to sphere of the left eye) has a first threshold of −1.25, an AUC of 0.51, and a number of SNPs of 13.

Submodel II (screening criteria: log 10(BF)>1.5, a second SNP site combination related to sphere of the left eye) has a first threshold of −3.75, an AUC of 0.55, and a number of SNPs of 95.

Submodel III (screening criteria: log 10(BF)>1, a third SNP site combination related to sphere of the left eye) has a first threshold of −3.25, an AUC of 0.51, and a number of SNPs of 333.

In one example, the genome-wide association of a total of 89,095 common variants in 8961 high myopia samples with a quantitative trait: cylinder of the left eye was obtained by an SNPTEST method, and then snps were screened by 4 thresholds for modeling: log 10(BF)>3; log 10(BF)>2; log 10(BF)>1.5; and log 10(BF)>1, resulting in a total of 429 snps. The submodel of cylinder of the left eye includes one or more of the following submodels: submodel I, submodel II, submodel III, and submodel IV.

Submodel I (screening criteria: log 10(BF)>3, a first SNP site combination related to cylinder of the left eye) has a first threshold of −2.5, an AUC of 0.56, and a number of SNPs of 7.

Submodel II (screening criteria: log 10(BF)>2, a second SNP site combination related to cylinder of the left eye) has a first threshold of −3.75, an AUC of 0.68, and a number of SNPs of 45.

Submodel III (screening criteria: log 10(BF)>1.5, a third SNP site combination related to cylinder of the left eye) has a first threshold of −3.75, an AUC of 0.73, and a number of SNPs of 130.

Submodel IV (screening criteria: log 10(BF)>1, a fourth SNP site combination related to cylinder of the left eye) has a first threshold of −4.25, an AUC of 0.77, and a number of SNPs of 429.

Then, the initial target single nucleotide polymorphism data are extracted using Python to obtain extracted initial target single nucleotide polymorphism data, wherein the initial target single nucleotide polymorphism data is a vcf file.

Finally, the extracted initial target single nucleotide polymorphism data is input and an additive model is selected, thereby obtaining an original genotype profile for the initial target single nucleotide polymorphism data.

Optionally, the method or step of selecting target single nucleotide polymorphism data by screening from the single nucleotide polymorphism data as characteristic data further comprises a method or step after obtaining an original genotype profile for the initial target single nucleotide polymorphism data: inputting the original genotype profile for the initial target single nucleotide polymorphism data, calculating a number of missing data of the single nucleotide polymorphism data in a sample, and performing removing or imputation on original initial target single nucleotide polymorphism data corresponding to the original genotype profile for the initial target single nucleotide polymorphism data according to the number of missing data to obtain the target single nucleotide polymorphism data.

Optionally, the SNPTEST method includes, but is not limited to, the following methods: Bayesian tests.

Optionally, in the additive model, no mutation is marked as 0, mutation with one allele is marked as 1, and mutation with two alleles is marked as 2; for the additive model, the formula is:

$${y}_{i} = β {G}_{i} + {e}_{i} , {e}_{i} ∼ N ( 0 , {σ}^{2} )$$

yi denotes a residual phenotype of the ith individual. The residual phenotype is calculated by subtracting one baseline term and estimating any specified covariates. Gi denotes an additive code for the expected genotype of the ith individual, wherein wild homozygous genotype=“0”, heterozygous genotype=“1”, and mutant homozygous genotype=“2”.

σ2 denotes the error variance of the model. σ²˜IG(a, b), β˜N(m_{β}, V_{β}σ²), and by default, a=3, b=2, m_{β}=0,V_{β}=0.02. Age and sex are used as covariates for correction.

Optionally, the processing standard for performing removing or imputation on initial target single nucleotide polymorphism data is 5%, and 5% is the result of N_missing/N_allsamples; among single samples, for samples with a number of missing data of single nucleotide polymorphism data of >5% in a single sample, a sample removing step is run; for samples with a number of missing data of single nucleotide polymorphism data of <5%, a imputation step is run; the artificial neural network has three layers including an input layer, a hidden layer, and an output layer: the number of nodes in the input layer is len(label_data), that is, there is no missing snp at all; the hidden layer is provided with 20 nodes; the output layer is provided with 3 nodes; the learning rate is set to 0.003 and the number of training iterations is set to 20.

Optionally, the target single nucleotide polymorphism data are highly specific and sensitive SNPs significantly related to myopia.

In one example, the myopia model comprises one or more of the following 9 myopia models: a myopia or high myopia model, a model of spherical equivalent difference between the two eyes, a model of spherical equivalent of the worst eye, a model of spherical equivalent of the right eye, a model of sphere of the right eye, a model of cylinder of the right eye, a model of spherical equivalent of the left eye, a model of sphere of the left eye, and a model of cylinder of the left eye,

In one example, the myopia model obtains a classification result for the myopia risk and/or myopia degree of a test sample through characteristic data of one or more combinations in the target single nucleotide polymorphism data.

In one example, the method or step of inputting the single nucleotide polymorphism data of the test sample into a myopia model for processing to obtain classification results for the single nucleotide polymorphism data of the test sample; and outputting a classification result for myopia of the test sample according to the classification results for the single nucleotide polymorphism data of the test sample comprises:
inputting the single nucleotide polymorphism data of the test sample into one or more of 9 myopia models to obtain classification results for the single nucleotide polymorphism data of the test sample in each of the myopia models; and
weighting classification results of the 9 myopia models according to the classification results for the single nucleotide polymorphism data of the test sample in each of the myopia models to obtain a classification result for myopia of the test sample.

Optionally, the single nucleotide polymorphism data of the test sample is input into a myopia or high myopia model, and classification results are output.

Optionally, the single nucleotide polymorphism data of the test sample is input into a model of spherical equivalent difference between the two eyes, and classification results in submodel I are output firstly; otherwise, the program is run to submodel II, and classification results in submodel II are output; otherwise, the program is run to submodel III, and classification results in submodel III are output; otherwise, the program is run to submodel IV, and classification results in submodel IV are output.

Optionally, the single nucleotide polymorphism data of the test sample is input into a model of spherical equivalent of the worst eye, and classification results in submodel I are output firstly; otherwise, the program is run to submodel II, and classification results in submodel II are output; otherwise, the program is run to submodel III, and classification results in submodel III are output; otherwise, the program is run to submodel IV, and classification results in submodel IV are output.

Optionally, the single nucleotide polymorphism data of the test sample is input into a model of spherical equivalent of the right eye, and classification results in submodel I are output firstly; otherwise, the program is run to submodel II, and classification results in submodel II are output; otherwise, the program is run to submodel III, and classification results in submodel III are output; otherwise, the program is run to submodel IV, and classification results in submodel IV are output.

Optionally, the single nucleotide polymorphism data of the test sample is input into a model of sphere of the right eye, and classification results in submodel I are output firstly; otherwise, the program is run to submodel II, and classification results in submodel II are output; otherwise, the program is run to submodel III, and classification results in submodel III are output; otherwise, the program is run to submodel IV, and classification results of submodel IV are output.

Optionally, the single nucleotide polymorphism data of the test sample is input into a model of cylinder of the right eye, and classification results in submodel I are output firstly; otherwise, the program is run to submodel II, and classification results in submodel II are output; otherwise, the program is run to submodel III, and classification results in submodel III are output; otherwise, the program is run to submodel IV, and classification results of submodel IV are output.

Optionally, the single nucleotide polymorphism data of the test sample is input into a model of spherical equivalent of the left eye, and classification results in submodel I are output firstly; otherwise, the program is run to submodel II, and classification results in submodel II are output; otherwise, the program is run to submodel III, and classification results in submodel III are output.

Optionally, the single nucleotide polymorphism data of the test sample is input into a model of sphere of the left eye, and classification results in submodel I are output firstly; otherwise, the program is run to submodel II, and classification results in submodel II are output; otherwise, the program is run to submodel III, and classification results in submodel III are output.

Optionally, the single nucleotide polymorphism data of the test sample is input into a model of cylinder of the left eye, and classification results in submodel I are output firstly; otherwise, the program is run to submodel II, and classification results in submodel II are output; otherwise, the program is run to submodel III, and classification results in submodel III are output; otherwise, the program is run to submodel IV, and classification results in submodel IV are output.

Optionally, a classification linear model is used in the processing process, and the classification process may be a binary classification process or a multi-class classification process.

The classification process comprises the following steps:
1) according to the quantitative trait of spherical equivalent difference between the two eyes or any one of other 7 quantitative traits, taking different thresholds for modeling, investigating AUC values, and taking a threshold of a model with a maximum AUC as a threshold of a final classification model, wherein the rules are as follows: 0 all values of spherical equivalent difference between the two eyes are transversed and are each used as a threshold to divide samples into two groups; @ the number of samples in each group should not be less than 100; and
2) classifying the samples using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed (the samples were extracted 10 times, with N samples extracted each time, and trained to obtain a model, which was a stable model in the case that the result could be met—sampling), and an average of 10 results was taken as a model result to ensure the objectivity of the model.

In one example, the characteristic selection was performed in parallel by using a plurality of different machine learning methods to select characteristics in the model which result in the maximum accuracy. optionally, the machine learning method includes one or more of recursive feature elimination, CART, random forest, linear regression, naive Bayes, and a customized training model; optionally, the myopia model further comprises a regularization term.

### Example 2: SNP Sites Related to Myopia or High Myopia

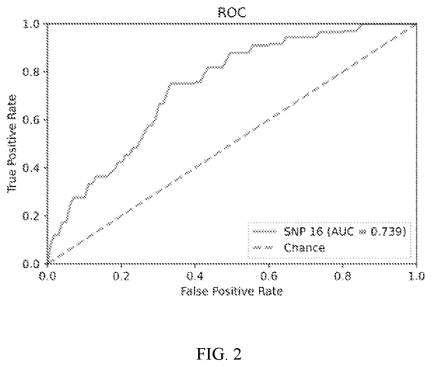

In one example, the genome-wide association of a total of 89,095 common variants in 8961 high myopia samples with myopia or high myopia was obtained by an SNPTEST method, and then snps were screened by a threshold of −6.01D for modeling: log 10(BF)>3, resulting in a total of 16 snps (detailed information is shown in Table 1), with an AUC of 0.739. The ROC curve is shown in FIG. 2.

TABLE 1
Information on 16 SNP sites related to myopia or high myopia

| RS ID | Chromosome | Position | Risk genotype | Other genotype | log10(BF) | Name of Gene | Position |
|---|---|---|---|---|---|---|---|
| rs78587889 | 1 | 110719412 | A | G | 3.01335 | SLC6A17 | intronic |
| rs41303763 | 16 | 24229699 | T | C | 3.67114 | PRKCB | UTR3 |
| rs2303221 | 16 | 31120772 | A | G | 3.07547 | BCKDK | intronic |
| rs80094829 | 1 | 151671355 | C | T | 4.63297 | SNX27 | UTR3 |
| rs1196455 | 1 | 151735545 | G | A | 3.65744 | OAZ3 | exonic |
| rs61746299 | 1 | 151773851 | A | T | 3.17396 | LINGO4 | exonic |
| rs3817585 | 3 | 11060430 | C | G | 3.07726 | SLC6A1 | ncRNA_intronic |
| rs2296651 | 14 | 70245193 | A | G | 3.07531 | SLC10A1 | exonic |
| rs2547319 | 19 | 51883149 | T | G | 3.33643 | LIM2 | downstream |
| rs143477571 | 14 | 76905712 | G | A | 3.05443 | ESRRB | |
| rs42688 | 5 | 127623170 | A | G | 3.1297 | FBN2 | intronic |
| rs189212243 | 7 | 158719791 | C | A | 5.00053 | WDR60 | intronic |
| rs143580489 | 6 | 62284356 | C | CA | 3.52953 | MTRNR2L9 | |
| (rs6915000) | | | | | | | |
| rs10484690 | 6 | 62442604 | G | A | 3.6402 | KHDRBS2 | exonic |
| rs26173 | 5 | 14479938 | G | A | 6.42994 | TRIO | intronic |
| rs117984746 | 21 | 46900356 | C | G | 3.24106 | COL18A1 | intronic |

### Example 3: SNP Sites Related to Spherical Equivalent Difference Between the Two Eyes

The data were analyzed according to the general method:

1. Selection of snps:

The genome-wide association of a total of 89,095 common variants in 8961 high myopia samples with a quantitative trait of spherical equivalent difference between the two eyes was obtained by using SNPTEST.

snps were screened by 4 thresholds for modeling: log 10(BF)>3; log 10(BF)>2; log 10(BF)>1.5; and log 10(BF)>1, resulting in a total of 341 snps.

2. Acquisition of Original Genotype Profile for snps:

The corresponding required snps (a total of 341 snps) in vcf were extracted by using python, an additive model (with no mutation marked as 0, a mutation with one allele marked as 1, and mutation with two alleles marked as 2) was selected, and an original genotype profile for snps was obtained.

3. Imputation of Genotype Profile for snps:

The number of missing data of snps in each of the samples in the original genotype profile for snps was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing snp at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of spherical equivalent difference between the two eyes, different thresholds were taken for modeling, AUC values were investigated, and a threshold of a model with a maximum AUC was taken as a threshold of a final classification model.

The rules are as follows:
\textcircled{1} all values of spherical equivalent difference between the two eyes are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} \frac{1}{2} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a skleamn.model_selection function, samples were divided into 10 groups of training and verification sets, a 10-fold cross-validation was performed, and an average of 10 results was taken as a model result to ensure the objectivity of the model.

The cutoff values and AUC values for the models are as follows:

TABLE 2
Changes in AUC at different cutoff values for each model

| | Spherical equivalent | Spherical equivalent | Spherical equivalent | Spherical equivalent |
|---|---|---|---|---|
| | difference between | difference between | difference between | difference between |
| | the two eyes | the two eyes | the two eyes | the two eyes |
| Threshold | (logbf > 3) | (logbf > 2) | (logbf > 1.5) | (logbf > 1) |
| 0 | 0.516741 | 0.546439 | 0.555604 | 0.542092 |
| 0.125 | 0.511118 | 0.527726 | 0.560627 | 0.564904 |
| 0.25 | 0.508225 | 0.526064 | 0.542424 | 0.565831 |
| 0.375 | 0.507513 | 0.532831 | 0.554618 | 0.58191 |
| 0.5 | 0.5017 | 0.530184 | 0.554205 | 0.582062 |
| 0.625 | 0.512903 | 0.53839 | 0.567589 | 0.595834 |
| 0.75 | 0.516728 | 0.544279 | 0.575803 | 0.608243 |
| 0.875 | 0.52321 | 0.542974 | 0.586271 | 0.617503 |
| 1 | 0.523663 | 0.546611 | 0.587548 | 0.628438 |
| 1.125 | 0.525407 | 0.551908 | 0.593181 | 0.640446 |
| 1.25 | 0.535277 | 0.559252 | 0.602594 | 0.656735 |
| 1.375 | 0.535771 | 0.565782 | 0.611966 | 0.662145 |
| 1.5 | 0.539699 | 0.573434 | 0.617433 | 0.666574 |
| 1.625 | 0.54236 | 0.573077 | 0.622267 | 0.674835 |
| 1.75 | 0.545405 | 0.580374 | 0.62699 | 0.681866 |
| 1.875 | 0.549351 | 0.589868 | 0.632628 | 0.681078 |
| 2 | 0.557354 | 0.598462 | 0.641929 | 0.687175 |
| 2.125 | 0.558264 | 0.601324 | 0.646573 | 0.691216 |
| 2.25 | 0.566933 | 0.612139 | 0.657544 | 0.702332 |
| 2.375 | 0.576226 | 0.618891 | 0.67228 | 0.722974 |
| 2.5 | 0.583319 | 0.629165 | 0.673219 | 0.723715 |
| 2.625 | 0.590088 | 0.633212 | 0.681649 | 0.730597 |
| 2.75 | 0.588349 | 0.630086 | 0.678357 | 0.739652 |
| 2.875 | 0.590382 | 0.641936 | 0.681936 | 0.751198 |
| 3 | 0.597796 | 0.647057 | 0.685131 | 0.751337 |
| 3.125 | 0.603008 | 0.651367 | 0.706319 | 0.765423 |
| 3.25 | 0.60088 | 0.655985 | 0.712332 | 0.768467 |
| 3.375 | 0.598911 | 0.665609 | 0.715675 | 0.783088 |
| 3.5 | 0.599908 | 0.674504 | 0.719677 | 0.787252 |
| 3.625 | 0.598101 | 0.677183 | 0.72111 | 0.797811 |
| 3.75 | 0.60233 | 0.681306 | 0.727545 | 0.815382 |
| 3.875 | 0.599182 | 0.684556 | 0.732102 | 0.819396 |
| 4 | 0.610049 | 0.694005 | 0.736019 | 0.806548 |
| 4.125 | 0.614171 | 0.70286 | 0.747145 | 0.811535 |
| 4.25 | 0.608196 | 0.699282 | 0.741584 | 0.809398 |
| 4.375 | 0.619153 | 0.714663 | 0.751073 | 0.810732 |
| 4.5 | 0.620481 | 0.717035 | 0.753018 | 0.810197 |
| 4.625 | 0.626125 | 0.722242 | 0.758685 | 0.809838 |
| 4.75 | 0.619938 | 0.721663 | 0.754338 | 0.814257 |
| 4.875 | 0.628387 | 0.712963 | 0.741965 | 0.801946 |
| 5 | 0.642494 | 0.718385 | 0.73834 | 0.796262 |
| 5.125 | 0.643409 | 0.728701 | 0.774276 | 0.827877 |
| 5.25 | 0.647591 | 0.738394 | 0.782254 | 0.851713 |
| 5.375 | 0.659725 | 0.740486 | 0.788708 | 0.863553 |
| 5.5 | 0.660996 | 0.732854 | 0.782205 | 0.86925 |
| 5.625 | 0.677987 | 0.729189 | 0.772439 | 0.883713 |

The following 5 SNPs were obtained by screening with log bf>3 as criteria:

rs80094829, rs1196455, rs61746299, rs3817585, and rs2296651.

The following 34 SNPs were obtained by screening with log bf>2 as criteria:

rs138802237, rs80094829, rs1196455, rs61746299, rs17846715, rs77819192, rs74711761, rs186128635, rs3817585, rs166508, rs11745612, rs164074, rs164075, rs164076, rs140779761, rs17652080, rs17708515, rs17110944, rs140691873, rs1042454, rs1613776, rs57344881, rs2296651, rs2302230, rs2302231, rs4147910, rs74406853, rs199946960, rs35689937, rs34378744, rs71357953, rs71357954, rs201326575, and rs2075937.

The following 110 SNPs were obtained by screening with log bf>1.5 as criteria:

rs183232121, rs138802237, rs7539625, rs80094829, rs1196455, rs61746299, rs150026164, rs12043009, rs17846715, rs4653415, rs77819192, rs117238208, rs34117315, rs4851287, rs74711761, rs13422424, rs186128635, rs150129946, rs35678, rs2289272, rs3817585, rs166508, rs73135528, rs73135529, rs73135530, rs73135535, rs73135545, rs36077176, rs73135584, rs137919012, rs3813832, rs11745612, rs56969869, rs397741115_rs56191507, rs16899974, rs164073, rs164074, rs164075, rs164076, rs140779761, rs17652080, rs17708515, rs17110944, rs6627, rs61748571, rs137899339, rs117192504, rs1109208, rs73689406, rs192085105, rs1830035, rs1830036, rs17138865, rs17545405, rs140691873, rs17553698, rs3810925, rs111748634, rs111841972, rs4073310, rs2271460, rs1042454, rs1044476, rs2274351, rs1613776, rs57344881, rs76274604, rs694066, rs7123133, rs7123272, rs28734900, rs12797894, rs17102543, rs1050395, rs181464388, rs2296651, rs3818188, rs1455773, rs13380006, rs199816463, rs429744, rs370627229, rs3743600, rs138793855, rs35896669, rs2302230, rs2302231, rs2907373, rs116067668, rs138221111, rs3752229, rs3764648, rs4147910, rs4147914, rs80054150, rs150907742, rs200222185, rs74406853, rs199946960, rs35689937, rs34378744, rs6511379, rs71357953, rs71357954, rs28678477, rs201326575, rs874100, rs1534879, rs2075937, and rs2239749.

The following 341 SNPs were obtained by screening with log bf>1 as criteria:

rs12133253, rs1573753, rs7512414, rs34109355, rs34976233, rs35013594, rs7528572, rs1541185, rs146160422, rs41303641, rs41311274, rs183232121, rs75321043, rs79777494, rs138802237, rs7664, rs1884444, rs7539625, rs76475726, rs599839, rs12083584, rs374505350, rs80094829, rs1196455, rs61746299, rs200519781, rs150026164, rs12043009, rs1375211271_rs373796693, rs17846707, rs17846715, rs3747626, rs1301522897_rs397775888, rs17846717, rs17846718, rs41288127, rs12087593, rs17380127, rs17380141, rs77202104, rs4653415, rs77819192, rs117238208, rs28383065, rs3731572, rs4148186, rs151242484, rs34117315, rs370613331, rs4851287, rs144642601, rs200736867, rs74711761, rs13422424, rs185464655, rs148663631, rs186128635, rs79028939, rs150129946, rs236843, rs10173558, rs150979437, rs140446783, rs56847463, rs183596892, rs117354149, rs35678, rs2075354, rs2289272, rs3817585, rs166508, rs370554533, rs78946357, rs73135513, rs73135528, rs73135529, rs73135530, rs73135535, rs73135545, rs36077176, rs73135584, rs117279588, rs137919012, rs17552950, rs151025946, rs77422573, rs3813832, rs4752, rs77676024, rs7687613, rs74991481, rs77885682, rs13152799, rs2276979, rs11745612, rs113222960, rs56969869, rs12523335, rs397741115_rs56191507, rs16899974, rs2560306, rs186414752, rs351260, rs164073, rs164074, rs164075, rs164076, rs140779761, rs17652080, rs17708515, rs17110944, rs111995800, rs3839291_rs398000092, rs6627, rs17136239, rs61748571, rs2815822, rs35374726, rs77229316, rs3094225, rs2281831, rs113689068, rs137899339, rs17710008, rs7763726, rs117192504, rs186624179, rs2292498, rs3763384, rs146123998, rs17779352, rs192189247, rs1109208, rs73689406, rs192085105, rs1830035, rs1830036, rs17138865, rs17545405, rs41350445, rs34587586, rs78637678, rs4503083, rs188235566, rs140691873, rs79930604, rs41297187, rs41307479, rs17553698, rs3810925, rs111748634, rs111841972, rs7850697, rs2231406, rs4073310, rs2271460, rs80042909, rs11200920, rs11200933, rs1042454, rs1934951, rs1044476, rs11191295, rs7081110, rs7081467, rs2274351, rs1613776, rs3135802, rs6693, rs183813046, rs57344881, rs907610, rs80326119, rs76274604, rs77600363, rs486584, rs12804382, rs12805133, rs201373803, rs694066, rs189385634, rs149080490, rs138619667, rs7123133, rs7123272, rs28734900, rs12797894, rs7307854, rs76066543, rs139938131, rs7980396, rs3217157 rs35118302 rs537549359, rs149247686, rs75656873, rs2277373, rs59261129, rs181052973, rs183437347, rs17102543, rs1050395, rs3751129, rs77973991, rs2036771, rs4759544, rs181464388, rs112163489, rs9594738, rs3210286, rs191708663, rs149954510, rs440466, rs2331979, rs7159367, rs34598192, rs2277475, rs3729818, rs117109392, rs58154675, rs2295106, rs2295107, rs2235967, rs7143196, rs2296651, rs116897063, rs562694521, rs536155089, rs1884541, rs3818188, rs200138830, rs7177192, rs2412541, rs11858113, rs8041534, rs8040502, rs11070285, rs2412544, rs141891738, rs12232341, rs143563919, rs3743476, rs1455773, rs13380006, rs74642800, rs199816463, rs373684171, rs429744, rs370627229, rs78671991, rs529955941, rs41303763, rs11867123, rs7206481, rs3743600, rs11656203, rs138793855, rs115332545, rs3736238, rs147085136, rs11649834, rs140947135, rs8556, rs2303015, rs3826301, rs35896669, rs75743672, rs729782, rs59589773, rs184602743, rs2302230, rs2302231, rs2907373, rs140010427, rs3744032, rs2748428, rs73410309, rs11657217, rs148660676, rs1192271300_rs397858239, rs566655, rs116067668, rs138221111, rs189222917, rs375483921, rs3752229, rs3764648, rs4147910, rs4147914, rs1610096, rs2074442, rs117187726, rs2074454, rs80054150, rs150907742, rs4807578, rs143662386, rs28480458, rs139113505, rs200222185, rs74406853, rs199946960, rs35689937, rs34378744, rs6511379, rs71357953, rs71357954, rs1801015, rs80027112, rs4635, rs56276635, rs184733141, rs79863139, rs181599488, rs138399909, rs324125, rs8109631, rs80087697, rs200619037, rs28678477, rs2273423, rs6023533, rs5743513, rs817343, rs45566234, rs7281104, rs14194, rs201326575, rs874100, rs202195284, rs1894473, rs17670808, rs17738527, rs17738540, rs9606738, rs9608944, rs9608956, rs1534879, rs2075937, rs2239749, rs202112368, rs188655025, and rs11541025.

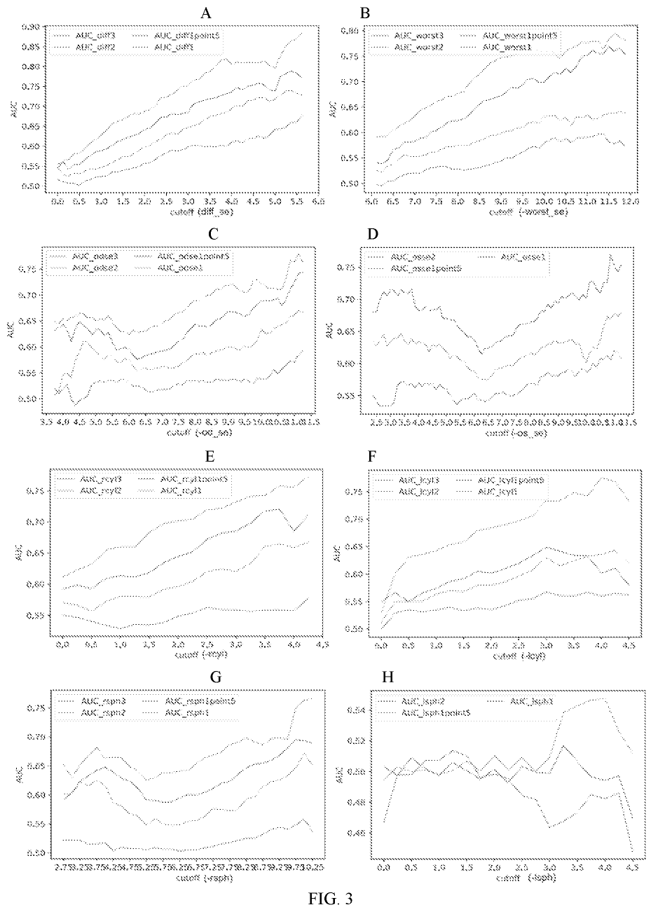

Then, the above models were optimized, the optimal thresholds (cutoff) were obtained by screening, and the ROC curves were plotted (as shown in Table 3 and FIG. 3).

TABLE 3
Optimal thresholds and AUC values for the models

| Model | Threshold | AUC | Number of SNPs |
|---|---|---|---|
| Model 1 | 5.625 | 0.66 | 5 SNPs obtained by screening |
| | | | with logbf > 3 as criteria |
| Model 2 | 5.375 | 0.74 | 34 SNPs obtained by screening |
| | | | with logbf > 2 as criteria |
| Model 3 | 5.5 | 0.80 | 110 SNPs obtained by screening |
| | | | with logbf > 1.5 as criteria |
| Model 4 | 5.625 | 0.88 | 341 SNPs obtained by screening |
| | | | with logbf > 1 as criteria |

That is, when 5 SNP sites obtained by screening with log bf>3 as criteria are used as markers, whether the spherical equivalent difference between the two eyes of a subject is above 5.625 can be effectively determined;
when 34 SNP sites obtained by screening with log bf>2 as criteria are used as markers, whether the spherical equivalent difference between the two eyes of a subject is above 5.375 can be effectively determined;
when 110 SNP sites obtained by screening with log bf>1.5 as criteria are used as markers, whether the spherical equivalent difference between the two eyes of a subject is above 5.5 can be effectively determined;
when 341 SNP sites obtained by screening with log bf>1 as criteria are used as markers, whether the spherical equivalent difference between the two eyes of a subject is above 5.625 can be effectively determined.

### Example 4: SNP Sites Related to Spherical Equivalent Difference of the Worst Eye

The data were analyzed according to the general method:

1. Selection of snps:

The genome-wide association of a total of 89,095 common variants in 8961 high myopia samples with a quantitative trait of spherical equivalent of the worst eye was obtained by using SNPTEST.

snps were screened by 4 thresholds for modeling: log 10(BF)>3; log 10(BF)>2; log 10(BF)>1.5; and log 10(BF)>1, resulting in a total of 317 snps.

2. Acquisition of Original Genotype Profile for snps:

The corresponding required snps (a total of 317 snps) in vcf were extracted by using python, an additive model (with no mutation marked as 0, a mutation with one allele marked as 1, and mutation with two alleles marked as 2) was selected, and an original genotype profile for snps was obtained.

3. Imputation of Genotype Profile for snps:

The number of missing data of snps in each of the samples in the original genotype profile for snps was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing snp at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of spherical equivalent of the worst eye, different thresholds were taken for modeling, AUC values were investigated, and a threshold of a model with a maximum AUC was taken as a threshold of a final classification model.

The rules are as follows:

\textcircled{1} all values of spherical equivalent of the worst eyes are transversed and are each used as a threshold to divide samples into two groups;

\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a skleamn.model_selection function, samples were divided into 10 groups of training and verification sets, a 10-fold cross-validation was performed, and an average of 10 results was taken as a model result to ensure the objectivity of the model.

The cutoff values and AUC values for the models are as follows:

TABLE 4
Changes in AUC at different cutoff values for each model

| | Spherical | Spherical | Spherical | Spherical |
|---|---|---|---|---|
| | equivalent of | equivalent of | equivalent of | equivalent of |
| | the worst eye | the worst eye | the worst eye | the worst eye |
| Threshold | (logbf > 3) | (logbf > 2) | (logbf > 1.5) | (logbf > 1) |
| −6.125 | 0.497211 | 0.524907 | 0.54046 | 0.591533 |
| −6.25 | 0.496065 | 0.521714 | 0.539 | 0.592703 |
| −6.375 | 0.504663 | 0.534239 | 0.545948 | 0.593134 |
| −6.5 | 0.50496 | 0.537959 | 0.566366 | 0.60457 |
| −6.625 | 0.512311 | 0.547535 | 0.568985 | 0.61141 |
| −6.75 | 0.516498 | 0.555652 | 0.581269 | 0.61753 |
| −6.875 | 0.520107 | 0.553379 | 0.581109 | 0.622664 |
| −7 | 0.520967 | 0.552928 | 0.582596 | 0.632339 |
| −7.125 | 0.520502 | 0.553775 | 0.588822 | 0.642984 |
| −7.25 | 0.526353 | 0.555615 | 0.590118 | 0.64744 |
| −7.375 | 0.530597 | 0.56152 | 0.601359 | 0.65355 |
| −7.5 | 0.531985 | 0.561777 | 0.603867 | 0.661118 |
| −7.625 | 0.534261 | 0.566881 | 0.610939 | 0.664252 |
| −7.75 | 0.532303 | 0.571527 | 0.618916 | 0.671051 |
| −7.875 | 0.527464 | 0.572126 | 0.622681 | 0.671539 |
| −8 | 0.529037 | 0.574539 | 0.62327 | 0.676973 |
| −8.125 | 0.526131 | 0.573735 | 0.626372 | 0.680026 |
| −8.25 | 0.52772 | 0.575317 | 0.633482 | 0.694997 |
| −8.375 | 0.529405 | 0.581416 | 0.646251 | 0.707682 |
| 8.5 | 0.530404 | 0.583871 | 0.655435 | 0.713457 |
| −8.625 | 0.53436 | 0.588307 | 0.663356 | 0.72488 |
| −8.75 | 0.536766 | 0.594079 | 0.6648 | 0.736766 |
| −8.875 | 0.541315 | 0.595215 | 0.669359 | 0.741671 |
| −9 | 0.543856 | 0.598977 | 0.672569 | 0.747479 |
| −9.125 | 0.550408 | 0.602793 | 0.679379 | 0.748454 |
| −9.25 | 0.549112 | 0.603761 | 0.688915 | 0.751246 |
| −9.375 | 0.556205 | 0.609007 | 0.686904 | 0.75483 |
| −9.5 | 0.561429 | 0.610161 | 0.693103 | 0.766592 |
| −9.625 | 0.567739 | 0.618588 | 0.704649 | 0.770221 |
| −9.75 | 0.572592 | 0.620922 | 0.706056 | 0.766666 |
| −9.875 | 0.569059 | 0.61913 | 0.698711 | 0.760439 |
| −10 | 0.58035 | 0.631184 | 0.699236 | 0.758523 |
| −10.125 | 0.585234 | 0.631982 | 0.710099 | 0.762354 |
| −10.25 | 0.59056 | 0.633462 | 0.717911 | 0.756112 |
| −10.375 | 0.582078 | 0.624653 | 0.714671 | 0.745155 |
| −10.5 | 0.588107 | 0.629651 | 0.725359 | 0.760965 |
| −10.625 | 0.580207 | 0.6137 | 0.717255 | 0.766021 |
| −10.75 | 0.589361 | 0.630912 | 0.730752 | 0.769987 |
| −10.875 | 0.590099 | 0.628717 | 0.749897 | 0.777671 |
| −11 | 0.590772 | 0.634033 | 0.751976 | 0.776977 |
| −11.125 | 0.592231 | 0.62579 | 0.761167 | 0.779061 |
| −11.25 | 0.597111 | 0.627361 | 0.755198 | 0.778935 |
| −11.375 | 0.596759 | 0.63466 | 0.755385 | 0.764125 |
| −11.5 | 0.581125 | 0.637427 | 0.769031 | 0.783733 |
| −11.625 | 0.578051 | 0.637176 | 0.759283 | 0.795135 |
| −11.75 | 0.583959 | 0.640691 | 0.767039 | 0.787831 |
| −11.875 | 0.574038 | 0.638385 | 0.75378 | 0.78086 |

The following 3 SNPs were obtained by screening with log bf>3 as criteria:

rs78587889, rs41303763, and rs2303221.

The following 22 SNPs were obtained by screening with log bf>2 as criteria:

rs78587889, rs188480146, rs2292661, rs6894260, rs4957836, rs148474510, rs200506987, rs145293869, rs2248077, rs2811736, rs2297722, rs2297723, rs9987876, rs7157977, rs41303763, rs2303221, rs145438063, rs151309111, rs6076122, rs2183573, rs9981301, and rs5756223.

The following 90 SNPs were obtained by screening with log bf>1.5 as criteria:

rs3103778, rs2746342, rs78587889, rs140141416, rs17024525, rs2288182, rs188480146, rs6734083, rs75054661, rs12492484, rs2292661, rs55947930, rs16837029, rs149213974, rs6894260, rs45625534, rs4957836, rs147518598, rs1042044, rs12671595, rs186526946, rs138414411, rs3802064, rs75456709, rs76427362, rs2073793, rs140518317, rs2214098, rs148474510, rs876087, rs200506987, rs8178175, rs145293869, rs200021348_rs552590290, rs16909677, rs2248077, rs2811736, rs2297722, rs2297723, rs9987876, rs34630110, rs141296329, rs117520659, rs3750996, rs3847553, rs142908193, rs1042669, rs12811832, rs78692679, rs11551723, rs7157977, rs3759779, rs3742770, rs530110551, rs3742464, rs3736911, rs11849022, rs77481241, rs3212112, rs9282879, rs55958706, rs3212056, rs114770841, rs3742368, rs563363438, rs2738893, rs1132358, rs41303763, rs2303221, rs145438063, rs151309111, rs139271842, rs533816037, rs305974, rs407731, rs406968, rs388706, rs2302948, rs2547319, rs7258847, rs4815467, rs41275624, rs6057110, rs6076122, rs4911402, rs2183573, rs9981301, rs4624474, rs5756223, and rs5845912.

The following 317 SNPs were obtained by screening with log bf>1 as criteria:

rs3795289, rs17033266, rs61785839, rs77649572, rs202140107, rs2275365, rs77275487, rs6676052, rs3103778, rs3014246, rs3014240, rs35016075_rs59669671, rs28375469, rs116861599, rs2746342, rs2457829, rs10889315, rs148811681, rs41305862, rs1410871, rs61729628, rs17553619, rs78587889, rs140141416, rs17024525, rs149097658, rs822441, rs183812847, rs112946310, rs12568050, rs12564283, rs75401237, rs10749643, rs2288182, rs400917, rs201752014, rs75526749, rs17189743, rs188480146, rs528123098, rs61748134, rs2228184, rs143686122, rs1434087, rs13002576, rs192688643, rs6734083, rs68104327, rs35996697, rs6437368, rs1077827, rs76657333, rs75054661, rs547922447, rs143654927, rs2286786, rs115250058, rs117336796, rs12492484, rs2292661, rs2276740, rs3774765, rs2245278, rs55947930, rs75144949, rs3732755, rs16837029, rs11732898, rs7696197, rs6841898, rs114264770, rs75058604, rs149213974, rs17001890, rs61732380, rs17029087, rs76996680, rs186309103, rs2303710, rs3749692, rs181303223, rs150821721, rs76381196, rs139298086_rs17839482, rs555615570, rs6894260, rs13189822, rs45625534, rs4957836, rs147518598, rs3829986, rs4608967, rs564109, rs150157174, rs2735059, rs4959012, rs757262, rs757259, rs117333063, rs185990098, rs41542812, rs11966699, rs4711571, rs4711572, rs2235868, rs1042044, rs3804502, rs2100375, rs118003870, rs75817012, rs17181352, rs35517174, rs17132399, rs2292498, rs3763384, rs12671595, rs186526946, rs138414411, rs13438494, rs3802064, rs75456709, rs76427362, rs2073793, rs140518317, rs2214098, rs79413133, rs2230156, rs148474510, rs41465145, rs876087, rs200506987, rs537284787, rs8178175, rs55991828, rs12549018, rs34961221, rs78240711, rs139669465, rs11144089, rs117169590, rs145293869, rs200021348_rs552590290, rs16909677, rs117175949, rs10481674, rs41276795, rs141549766, rs147026086, rs188197626, rs7038042, rs11849, rs2275161, rs10781513, rs2248077, rs2811736, rs2297722, rs2297723, rs9987876, rs7079747, rs34630110, rs148582275, rs4745933, rs111835023, rs185249748, rs117768118, rs141296329, rs117520659, rs45498396, rs3750996, rs183484, rs9937, rs2304733, rs147852038, rs139692587, rs10792769, rs2298573, rs4753069, rs3847553, rs2962118, rs142908193, rs1042669, rs11047401, rs12811832, rs11168338, rs2277373, rs1150057, rs1799986, rs1050395, rs78692679, rs3835190, rs201814780, rs11551723, rs7992732, rs7157977, rs10143899, rs3759779, rs7142098, rs118004333, rs3742770, rs3742768, rs3742767, rs143477571, rs530110551, rs3742464, rs3736911, rs11849022, rs77481241, rs3212112, rs9282879, rs55958706, rs3212056, rs34100926, rs114770841, rs3742368, rs4390564, rs72486624, rs7172888, rs563363438, rs12904657, rs4904, rs72546313, rs2745136, rs2738893, rs1054645, rs1132358, rs79763179, rs3743807, rs55969568, rs9941128, rs41303763, rs2303221, rs11859725, rs113276760, rs145438063, rs11550470, rs145797873, rs11556634, rs56126318, rs16954357, rs397718340_rs5819492, rs9899785, rs375644567, rs185576254, rs183898893, rs4986765, rs146684394, rs181969864, rs2242273, rs2170297, rs11081410, rs7233770, rs7235093, rs7236574, rs11659762, rs116067668, rs11538683, rs55646937, rs148018996, rs1143698, rs10412834, rs11260004, rs11882197, rs2288415, rs150461309, rs3746295, rs151309111, rs3810209, rs4808643, rs139271842, rs200222185, rs189120571, rs148003617, rs533816037, rs189041504, rs305968, rs305974, rs407731, rs406968, rs388706, rs2302948, rs182509214, rs198977, rs2547319, rs2547318, rs7258847, rs4815467, rs41275624, rs6057110, rs6076122, rs4911402, rs114985890, rs80158178, rs3746488, rs76747058, rs143199162, rs77432560, rs116846998, rs2183573, rs1984748, rs9981301, rs4624474, rs2838379, rs4566449, rs915770, rs2870984, rs2073733, rs41281587, rs3747135, rs3747136, rs3747137, rs5756223, rs3747168, rs369859899, rs6006901, rs5845912, rs9628315, and rs8137790.

Then, the above models were optimized, the optimal thresholds (cutoff) were obtained by screening, and the ROC curves were plotted (as shown in Table 5 and FIG. 3).

TABLE 5
Optimal thresholds and AUC values for the models

| Model | Threshold | AUC | Number of SNPs |
|---|---|---|---|
| Model 1 | −11.375 | 0.59 | 3 SNPs obtained by screening |
| | | | with logbf > 3 as criteria |
| Model 2 | −11.875 | 0.66 | 22 SNPs obtained by screening |
| | | | with logbf > 2 as criteria |
| Model 3 | −11.5 | 0.77 | 90 SNPs obtained by screening |
| | | | with logbf > 1.5 as criteria |
| Model 4 | −11.5 | 0.79 | 317 SNPs obtained by screening |
| | | | with logbf > 1 as criteria |

That is, when 3 SNP sites obtained by screening with log bf>3 as criteria are used as markers, whether the spherical equivalent of the worst eye of a subject is above −11.375 can be effectively determined;
when 22 SNP sites obtained by screening with log bf>2 as criteria are used as markers, whether the spherical equivalent of the worst eye of a subject is above −11.875 can be effectively determined;
when 90 SNP sites obtained by screening with log bf>1.5 as criteria are used as markers, whether the spherical equivalent of the worst eye of a subject is above −11.5 can be effectively determined;
when 317 SNP sites obtained by screening with log bf>1 as criteria are used as markers, whether the spherical equivalent of the worst eye of a subject is above −11.5 can be effectively determined.

### Example 5: SNP Sites Related to Spherical Equivalent of the Right Eye

The data were analyzed according to the general method:

1. Selection of snps:

The genome-wide association of a total of 89,095 common variants in 8961 high myopia samples with a quantitative trait of spherical equivalent of the right eye was obtained by using SNPTEST.

snps were screened by 4 thresholds for modeling: log 10(BF)>3; log 10(BF)>2; log 10(BF)>1.5; and log 10(BF)>1, resulting in a total of 342 snps.

2. Acquisition of Original Genotype Profile for snps:

The corresponding required snps (a total of 342 snps) in vcf were extracted by using python, an additive model (with no mutation marked as 0, a mutation with one allele marked as 1, and mutation with two alleles marked as 2) was selected, and an original genotype profile for snps was obtained.

3. Imputation of Genotype Profile for snps:

The number of missing data of snps in each of the samples in the original genotype profile for snps was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing snp at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of spherical equivalent of the right eye, different thresholds were taken for modeling, AUC values were investigated, and a threshold of a model with a maximum AUC was taken as a threshold of a final classification model.

The rules are as follows:
\textcircled{1} all values of spherical equivalent of the right eye are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed, and an average of 10 results was taken as a model result to ensure the objectivity of the model.

The cutoff values and AUC values for the models are as follows:

TABLE 6
Changes in AUC at different cutoff values for each model

| | Spherical | Spherical | Spherical | Spherical |
|---|---|---|---|---|
| | equivalent of | equivalent of | equivalent of | equivalent of |
| | the right eye | the right eye | the right eye | the right eye |
| Threshold | (logbf > 3) | (logbf > 2) | (logbf > 1.5) | (logbf > 1) |
| −3.75 | 0.521431 | 0.505949 | 0.630293 | 0.650236 |
| −3.875 | 0.509681 | 0.515924 | 0.637742 | 0.644106 |
| −4 | 0.520917 | 0.545801 | 0.643532 | 0.656167 |
| −4.125 | 0.527746 | 0.549083 | 0.63226 | 0.650466 |
| −4.25 | 0.500345 | 0.54132 | 0.609938 | 0.657367 |
| −4.375 | 0.486406 | 0.573806 | 0.635636 | 0.658421 |
| −4.5 | 0.497035 | 0.59223 | 0.648319 | 0.665831 |
| −4.625 | 0.500696 | 0.611198 | 0.640544 | 0.663547 |
| −4.75 | 0.50587 | 0.609545 | 0.636398 | 0.656012 |
| −4.875 | 0.528561 | 0.59756 | 0.623065 | 0.656895 |
| −5 | 0.531347 | 0.58918 | 0.634238 | 0.650926 |
| −5.125 | 0.53514 | 0.58592 | 0.63375 | 0.654799 |
| −5.25 | 0.531862 | 0.576562 | 0.622661 | 0.652496 |
| −5.375 | 0.536288 | 0.586506 | 0.628354 | 0.661075 |
| 5.5 | 0.53555 | 0.577203 | 0.60726 | 0.647814 |
| −5.625 | 0.5328 | 0.572325 | 0.594573 | 0.636699 |
| −5.75 | 0.53704 | 0.568139 | 0.596268 | 0.629827 |
| −5.875 | 0.538374 | 0.5776 | 0.599264 | 0.629713 |
| −6 | 0.529288 | 0.573504 | 0.595397 | 0.623924 |
| −6.125 | 0.524245 | 0.56356 | 0.584131 | 0.633269 |
| −6.25 | 0.525485 | 0.551084 | 0.576041 | 0.627738 |
| −6.375 | 0.522551 | 0.558837 | 0.578868 | 0.629655 |
| −6.5 | 0.517159 | 0.559314 | 0.583425 | 0.63121 |
| −6.625 | 0.51251 | 0.55576 | 0.585996 | 0.635581 |
| −6.75 | 0.513965 | 0.559044 | 0.587937 | 0.640652 |
| −6.875 | 0.517481 | 0.559633 | 0.590901 | 0.639459 |
| −7 | 0.517594 | 0.562632 | 0.592223 | 0.64715 |
| −7.125 | 0.517986 | 0.561172 | 0.593932 | 0.651207 |
| −7.25 | 0.519665 | 0.558604 | 0.596909 | 0.65342 |
| −7.375 | 0.525655 | 0.560604 | 0.602941 | 0.662486 |
| −7.5 | 0.529516 | 0.562723 | 0.61089 | 0.666023 |
| −7.625 | 0.526698 | 0.569655 | 0.615592 | 0.667244 |
| −7.75 | 0.534583 | 0.574055 | 0.627292 | 0.674054 |
| −7.875 | 0.534289 | 0.581913 | 0.635634 | 0.684886 |
| −8 | 0.53472 | 0.586373 | 0.643915 | 0.690117 |
| −8.125 | 0.533334 | 0.587538 | 0.639563 | 0.687011 |
| −8.25 | 0.535205 | 0.589137 | 0.640228 | 0.694714 |
| −8.375 | 0.532041 | 0.583502 | 0.638627 | 0.700446 |
| −8.5 | 0.532142 | 0.589124 | 0.642829 | 0.700879 |
| −8.625 | 0.537951 | 0.592047 | 0.652621 | 0.712315 |
| −8.75 | 0.5366 | 0.594135 | 0.654019 | 0.710355 |
| −8.875 | 0.536316 | 0.596299 | 0.660133 | 0.71769 |
| −9 | 0.535571 | 0.600578 | 0.668381 | 0.719305 |
| −9.125 | 0.537703 | 0.59926 | 0.667716 | 0.720865 |
| −9.25 | 0.536766 | 0.595956 | 0.666066 | 0.714258 |
| −9.375 | 0.533345 | 0.593922 | 0.662964 | 0.708973 |
| −9.5 | 0.538152 | 0.601707 | 0.659112 | 0.705124 |
| −9.625 | 0.533589 | 0.5989 | 0.662764 | 0.708327 |
| −9.75 | 0.53001 | 0.603847 | 0.674067 | 0.72662 |
| −9.875 | 0.539507 | 0.613207 | 0.681242 | 0.730787 |
| −10 | 0.538678 | 0.633463 | 0.688274 | 0.722545 |
| −10.125 | 0.538406 | 0.626011 | 0.689786 | 0.716073 |
| −10.25 | 0.54361 | 0.626541 | 0.688445 | 0.712788 |
| −10.375 | 0.556031 | 0.635796 | 0.691066 | 0.71406 |
| −10.5 | 0.548403 | 0.639289 | 0.693331 | 0.710988 |
| −10.625 | 0.557394 | 0.643381 | 0.678205 | 0.711394 |
| −10.75 | 0.565975 | 0.647486 | 0.692902 | 0.73368 |
| −10.875 | 0.572851 | 0.657828 | 0.711756 | 0.767697 |
| −11 | 0.570325 | 0.661601 | 0.724684 | 0.763846 |
| −11.125 | 0.586631 | 0.670405 | 0.741538 | 0.779396 |
| −11.25 | 0.593742 | 0.666119 | 0.744187 | 0.763031 |

rs2547319 was obtained by screening with log bf>3 as criteria.

The following 26 SNPs were obtained by screening with log bf>2 as criteria:

rs10889315, rs75144949, rs149213974, rs754615, rs45583335, rs2529438, rs2072236, rs2240403, rs78155900, rs114097201, rs2811736, rs2297722, rs2297723, rs117519669, rs7157977, rs10143899, rs3759779, rs143477571, rs2303221, rs34693726, rs3803752, rs142921938, rs3746295, rs2547319, rs2547318, and rs4911402.

The following 102 SNPs were obtained by screening with log bf>1.5 as criteria:

rs4648600, rs1061624, rs2275365, rs35909785, rs3103778, rs2457829, rs10889315, rs78587889, rs12568050, rs12564283, rs528123098, rs139842833, rs74953908, rs1077827, rs12492484, rs75144949, rs3732755, rs3135115, rs16837029, rs149213974, rs17001890, rs56382032, rs12651031, rs2736100, rs7724759, rs754615, rs5369, rs150157174, rs130068, rs45583335, rs78342561, rs2529438, rs1049402, rs2072236, rs2527878, rs2240403, rs78155900, rs114097201, rs2680903, rs59612871, rs3818584, rs16909677, rs10983755, rs2248077, rs2811736, rs2297722, rs2297723, rs9987876, rs7904554, rs907609, rs117519669, rs3750996, rs3802799, rs2276296, rs139692587, rs904628, rs11551723, rs144796593, rs78648016, rs7157977, rs10143899, rs3759779, rs7142098, rs143477571, rs3736911, rs11849022, rs77481241, rs3212112, rs9282879, rs55958706, rs3212056, rs34100926, rs114770841, rs3742368, rs118146919, rs2289702, rs2303221, rs34693726, rs3744566, rs9907420, rs375644567, rs185576254, rs3803752, rs17822735, rs17222523, rs34818467, rs12946397, rs3809724, rs181969864, rs7236574, rs142921938, rs3746295, rs151309111, rs533816037, rs2302948, rs182509214, rs2547319, rs2547318, rs2281209, rs4911402, rs138894461, and rs5756223.

The following 342 SNPs were obtained by screening with log bf>1 as criteria:

rs16824585, rs4648600, rs17033266, rs1061624, rs202140107, rs2291804, rs2275365, rs139241751, rs35909785, rs6676052, rs3103778, rs116893711, rs116861599, rs2457829, rs10889315, rs17855078, rs78587889, rs140141416, rs4656197, rs45445497, rs143355122, rs12568050, rs12564283, rs10482, rs1929861, rs75401237, rs28517482, rs1264208208, rs2631840, rs188480146, rs528123098, rs61732269, rs367931009, rs139842833, rs79908358, rs79358798, rs1434087, rs4894048, rs13421990, rs13419085, rs143196218, rs139713392, rs6734083, rs150887565, rs4663795, rs74953908, rs143001518, rs6437368, rs147838565, rs1077827, rs144623840, rs78233496, rs9876921, rs6622, rs2279909, rs547922447, rs300977, rs12492484, rs75144949, rs3732755, rs3135115, rs16837029, rs74787220, rs527903313, rs149213974, rs17001890, rs79769348, rs10025654, rs76996680, rs2306369, rs79482316, rs56382032, rs12651031, rs2736100, rs35130836, rs3749684, rs7724759, rs754615, rs45625534, rs3805625, rs137898880, rs147518598, rs202007699, rs2270900, rs75164992, rs17133512, rs76720430, rs5369, rs150157174, rs7756481, rs117298661, rs117644703, rs3094672, rs4713420, rs130068, rs67196728, rs185990098, rs9349593, rs143580489, rs3757302, rs3218602, rs9495370, rs45583335, rs78342561, rs117358940, rs9689983, rs2529438, rs1049402, rs2072236, rs2527878, rs2240403, rs781980620, rs56083327, rs2245368, rs2430307, rs3748126, rs36028884, rs78155900, rs114097201, rs13438494, rs117211799, rs115734214, rs148474510, rs200506987, rs8190955, rs8178175, rs2680903, rs117676215, rs115507207, rs369806130, rs78240711, rs59612871, rs117169590, rs3739523, rs145293869, rs139212809, rs3818584, rs16909677, rs141549766, rs147026086, rs188197626, rs13321, rs10983755, rs2301576, rs7038042, rs11849, rs2275161, rs2248077, rs2811736, rs2297722, rs2297723, rs9987876, rs7904554, rs148582275, rs3740168, rs149647444, rs7100474, rs7079648, rs202166096, rs60420182, rs36104328, rs7098255, rs3814163, rs2297785, rs3736924, rs3736923, rs141296329, rs117520659, rs57285449, rs112687793, rs73401681, rs79948463, rs10902170, rs10902171, rs867839, rs907608, rs907609, rs117519669, rs3750996, rs214086, rs3802799, rs76215382, rs147852038, rs2276296, rs143930161, rs183113103, rs139692587, rs10792769, rs7102675, rs116878689, rs35466364, rs142908193, rs7966459, rs7135745, rs34181394, rs1921038, rs201814780, rs11068531, rs904628, rs11551723, rs7983667, rs144796593, rs140526815_rs77878407, rs78646508, rs34523940, rs9521906, rs78648016, rs7157977, rs10143899, rs3759779, rs7142098, rs41309252, rs535020964, rs3742770, rs3742768, rs116881452, rs143477571, rs61992274, rs142973720, rs530110551, rs3742464, rs3736911, rs11849022, rs77481241, rs3212112, rs9282879, rs55958706, rs3212056, rs34100926, rs114770841, rs3742368, rs144857261_rs373032548, rs189734192, rs188393827, rs118146919, rs562829514, rs1805025, rs8192352, rs2289702, rs150921132, rs61869621, rs1802752, rs113355794, rs7198064, rs2291407, rs9941128, rs9941210, rs9941215, rs9931527, rs12708649, rs13306653, rs11643517, rs5723, rs7196736, rs2303221, rs2304483, rs149645127, rs11550470, rs17232091, rs150889000, rs16954357, rs371756524_rs557159990, rs192103504, rs115314145, rs34693726, rs2277651, rs2277652, rs71360269, rs71360270, rs3744566, rs3744568, rs9907420, rs375644567, rs185576254, rs2158460, rs3803752, rs17822735, rs17222523, rs34818467, rs12946397, rs3809724, rs35476409_rs397978887, rs3744214, rs181969864, rs4789773, rs28561791, rs7236574, rs76597875, rs61749945, rs118033234, rs1052696, rs1052692, rs148018996, rs34488963, rs3745379, rs3745385, rs142921938, rs3746295, rs151309111, rs139271842, rs140566569, rs533816037, rs78237760, rs79621739, rs75990051, rs62107869, rs305974, rs1055099, rs41290128, rs76151738, rs16980091, rs2302948, rs182509214, rs2547319, rs2547318, rs16982743, rs2284385, rs2281209, rs4911402, rs80158178, rs7509972, rs150511237, rs2231391, rs2231390, rs762228, rs72564613, rs191574093, rs138894461, rs5999924, rs5750146, rs5756223, rs3747168, rs148345118, rs910796, rs910797, rs910798, rs738712, rs16445, rs3922872, rs56248708, rs9628315, and rs8137790.

Then, the above models were optimized, the optimal thresholds (cutoff) were obtained by screening, and the ROC curves were plotted (as shown in Table 7 and FIG. 3).

TABLE 7
Optimal thresholds and AUC values for the models

| Model | Threshold | AUC | Number of SNPs |
|---|---|---|---|
| Model 1 | −11.125 | 0.59 | 1 SNP obtained by screening |
| | | | with logbf > 3 as criteria |
| Model 2 | −11.125 | 0.67 | 26 SNPs obtained by screening |
| | | | with logbf > 2 as criteria |
| Model 3 | −11.25 | 0.77 | 102 SNPs obtained by screening |
| | | | with logbf > 1.5 as criteria |
| Model 4 | −11.125 | 0.80 | 342 SNPs obtained by screening |
| | | | with logbf > 1 as criteria |

That is, when 1 SNP site obtained by screening with log bf>3 as criteria is used as a marker, whether the spherical equivalent of the right eye of a subject is above −11.125 can be effectively determined;
when 26 SNP sites obtained by screening with log bf>2 as criteria are used as markers, whether the spherical equivalent of the right eye of a subject is above −11.125 can be effectively determined;
when 102 SNP sites obtained by screening with log bf>1.5 as criteria are used as markers, whether the spherical equivalent of the right eye of a subject is above −11.25 can be effectively determined;
when 342 SNP sites obtained by screening with log bf>1 as criteria are used as markers, whether the spherical equivalent of the right eye of a subject is above −11.125 can be effectively determined.

### Example 6: SNP Sites Related to Sphere of the Right Eye

The data were analyzed according to the general method:

### 1. Selection of SNPs:

The genome-wide association of a total of 89,095 common variants in 8961 high myopia samples with a quantitative trait of sphere of the right eye (rsph) was obtained by using SNPTEST.

SNPs were screened by 4 thresholds for modeling: log 10(BF)>3; log 10(BF)>2; log 10(BF)>1.5; and log 10(BF)>1.

### 2. Acquisition of Original Genotype Profile for SNPs:

The corresponding required SNPs in vcf were extracted by using python, an additive model (with no mutation marked as 0, a mutation with one allele marked as 1, and mutation with two alleles marked as 2) was selected, and an original genotype profile for SNPs was obtained.

### 3. Imputation of Genotype Profile for SNPs:

The number of missing data of SNPs in each of the samples in the original genotype profile for SNPs was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing SNP at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of sphere of the right eye (rsph), different thresholds were taken for modeling, AUC values were investigated, and a threshold of a model with a maximum AUC was taken as a threshold of a final classification model.

The rules are as follows:
\textcircled{1} all values of sphere of the right eye (rsph) are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed, and an average of 10 results was taken as a model result to ensure the objectivity of the model. The cutoff values and AUC values for the models are shown in Table 8.

TABLE 8
Cutoff values and AUC values for the models

| | Sphere of | Sphere of | Sphere of | Sphere of |
|---|---|---|---|---|
| | the right eye | the right eye | the right eye | the right eye |
| Threshold | (logbf > 3) | (logbf > 2) | (logbf > 1.5) | (logbf > 1) |
| −2.75 | 0.522416 | 0.601938 | 0.592647 | 0.652381 |
| −3 | 0.522368 | 0.602168 | 0.602881 | 0.62994 |
| −3.25 | 0.52215 | 0.623317 | 0.623736 | 0.651096 |
| −3.5 | 0.515219 | 0.615702 | 0.632912 | 0.669079 |
| −3.75 | 0.515964 | 0.625421 | 0.642421 | 0.681663 |
| −4 | 0.51716 | 0.617071 | 0.647941 | 0.663395 |
| −4.25 | 0.504326 | 0.586861 | 0.639638 | 0.665148 |
| −4.5 | 0.509633 | 0.580454 | 0.628541 | 0.664149 |
| −4.75 | 0.507866 | 0.567495 | 0.623534 | 0.648517 |
| −5 | 0.507563 | 0.563467 | 0.610864 | 0.642016 |
| −5.25 | 0.505843 | 0.548552 | 0.591561 | 0.625978 |
| −5.5 | 0.509325 | 0.559629 | 0.590653 | 0.629678 |
| −5.75 | 0.506869 | 0.550582 | 0.587606 | 0.639073 |
| −6 | 0.50674 | 0.547669 | 0.588093 | 0.638663 |
| −6.25 | 0.503208 | 0.548459 | 0.595199 | 0.641294 |
| −6.5 | 0.505571 | 0.554848 | 0.600547 | 0.642472 |
| −6.75 | 0.50569 | 0.556065 | 0.600078 | 0.65394 |
| −7 | 0.508966 | 0.562909 | 0.604636 | 0.664175 |
| −7.25 | 0.511555 | 0.576717 | 0.610876 | 0.66907 |
| −7.5 | 0.515542 | 0.575327 | 0.620258 | 0.674049 |
| −7.75 | 0.517946 | 0.573049 | 0.62341 | 0.686211 |
| −8 | 0.521512 | 0.57453 | 0.63566 | 0.690887 |
| −8.25 | 0.525445 | 0.593259 | 0.646315 | 0.698659 |
| −8.5 | 0.525686 | 0.596924 | 0.653054 | 0.691898 |
| −8.75 | 0.531944 | 0.613661 | 0.654837 | 0.686029 |
| −9 | 0.54039 | 0.623151 | 0.659551 | 0.697228 |
| −9.25 | 0.544272 | 0.625727 | 0.668177 | 0.698317 |
| −9.5 | 0.541091 | 0.633376 | 0.680406 | 0.696354 |
| −9.75 | 0.54854 | 0.646945 | 0.69527 | 0.751356 |
| −10 | 0.558401 | 0.671499 | 0.69358 | 0.763488 |
| −10.25 | 0.53779 | 0.651282 | 0.689216 | 0.766073 |

In the above table, the SNP corresponding to Model 1 (log bf>3) is: rs143477571.

In the above table, the SNP combination corresponding to Model 2 (log bf>2) is:

rs1929861, rs139842833, rs149213974, rs130068, rs2072236, rs2240403, rs59612871, rs139692587, rs143477571, rs2303221, rs34693726, rs3744566, rs3803752, rs17223038, rs34488963, rs142921938, and rs2547319.

In the above table, the SNP combination corresponding to Model 3 (log bf>1.5) is:

rs35909785, rs10889315, rs143355122, rs10482, rs1929861, rs191149396, rs528123098, rs139842833, rs12492484, rs75144949, rs3732755, rs3135115, rs16837029, rs149213974, rs79769348, rs55656741, rs7724759, rs754615, rs137898880, rs147518598, rs75164992, rs17133512, rs76720430, rs3094672, rs4713420, rs130068, rs286560, rs2529438, rs2072236, rs2527878, rs2240403, rs56083327, rs2245368, rs2430307, rs3748126, rs36028884, rs117676215, rs115507207, rs2403730, rs59612871, rs3739523, rs2811736, rs2297722, rs2297723, rs202166096, rs2297785, rs3736924, rs3736923, rs117519669, rs57477011, rs139692587, rs10792769, rs142908193, rs140526815 (same as rs77878407), rs78646508, rs34523940, rs9521906, rs78648016, rs143477571, rs3742464, rs3736911, rs11849022, rs77481241, rs3212112, rs9282879, rs55958706, rs3212056, rs34100926, rs114770841, rs3742368, rs189734192, rs9941210, rs9941215, rs12708649, rs13306653, rs11643517, rs5723, rs2303221, rs11550470, rs2115400, rs150889000, rs192103504, rs2024076, rs34693726, rs2277648, rs2286357, rs12950718, rs2277651, rs2277652, rs71360269, rs71360270, rs3744566, rs3744568, rs3803752, rs17822735, rs17222523, rs34818467, rs12946397, rs3809724, rs3744214, rs7236574, rs1064058, rs1064059, rs17223038, rs34488963, rs3745379, rs3745385, rs142921938, rs3746295, rs2302948, rs2547319, rs2547318, rs4911402, and rs191574093.

In the above table, the SNP combination corresponding to Model 4 (log bf>1) is:

rs16824585, rs4648600, rs10864435, rs1061624, rs76538441, rs35909785, rs587404, rs3103778, rs10889315, rs17855078, rs78587889, rs140141416, rs117378923, rs143355122, rs12568050, rs12564283, rs10482, rs1929861, rs2275307, rs1339907598 (same as rs398050192), rs115095929, rs191149396, rs1264208208, rs574401502, rs11558179, rs2303607, rs528123098, rs61732269, rs367931009, rs3796110, rs139842833, rs79908358, rs74765462, rs57399379, rs143196218, rs139713392, rs28900695, rs74953908, rs147838565, rs125701, rs1801126, rs142876339, rs1969154, rs1985428, rs186408122, rs144623840, rs148545377, rs3749195, rs9876921, rs6622, rs2279909, rs724450, rs1138518, rs12492484, rs183217537, rs150448043, rs75144949, rs3732755, rs9857697, rs3135115, rs16837029, rs117253156, rs527903313, rs149213974, rs79769348, rs76996680, rs3828506, rs56382032, rs117171059, rs2736100, rs55656741, rs181303223, rs35130836, rs7724759, rs754615, rs137898880, rs147518598, rs202007699, rs13184586, rs2270900, rs2270898, rs75164992, rs17133512, rs139302155, rs76720430, rs5369, rs150157174, rs117298661, rs117644703, rs3094672, rs4713420, rs130068, rs9349593, rs2754779, rs75817012, rs45583335, rs78342561, rs117358940, rs78873069, rs286560, rs9689983, rs2529438, rs1049402, rs2072236, rs2527878, rs2240403, rs781980620, rs56083327, rs2245368, rs2430307, rs3748126, rs36028884, rs78155900, rs114097201, rs13438494, rs117211799, rs115734214, rs11556986, rs361330, rs2294127, rs2680903, rs117676215, rs115507207, rs114091176, rs11778661, rs2403730, rs7023474, rs78240711, rs149966849, rs139485366, rs59612871, rs117169590, rs3739523, rs200021348 (same as rs552590290), rs2274689, rs28446339, rs41306498, rs972925, rs2248077, rs2811736, rs2297722, rs2297723, rs9987876, rs7904554, rs148582275, rs7900977, rs35963845, rs11239594, rs202166096, rs60420182, rs140715254, rs2297785, rs3736924, rs3736923, rs117520659, rs907608, rs907609, rs117519669, rs16911845, rs3802799, rs662702, rs667773, rs57477011, rs61744209, rs76215382, rs147852038, rs2736597, rs1800007, rs143930161, rs183113103, rs139692587, rs10792769, rs2298573, rs7102675, rs56252953, rs116878689, rs10893102, rs74550564, rs35466364, rs215976, rs142908193, rs7966459, rs1921038, rs117866676, rs11551723, rs140728821 (same as rs74333174), rs7983667, rs144796593, rs140526815 (same as rs77878407), rs78646508, rs34523940, rs9521906, rs3211770, rs78648016, rs7157977, rs10143899, rs3759779, rs7142098, rs116881452, rs143477571, rs192594531, rs530110551, rs3742464, rs3736911, rs11849022, rs77481241, rs3212112, rs9282879, rs55958706, rs3212056, rs34100926, rs114770841, rs3742368, rs189734192, rs188393827, rs185222838, rs780162072, rs28381099, rs147960756, rs62032959, rs62033860, rs1802752, rs113355794, rs74988150, rs76534191, rs79609879, rs7198064, rs2291407, rs79763179, rs9941128, rs9941210, rs9941215, rs9931527, rs12708649, rs13306653, rs11643517, rs5723, rs7196736, rs71220135, rs202213258, rs2303221, rs117161621, rs11540994, rs149645127, rs117127365, rs13339651, rs11550470, rs2162943, rs2115400, rs4247353, rs3803690, rs3751696, rs75670228, rs150889000, rs16954357, rs192103504, rs2024076, rs12936716, rs34693726, rs2277648, rs2277649, rs12949680, rs2286357, rs12950718, rs2277651, rs2277652, rs71360269, rs71360270, rs3744566, rs3744568, rs4792008, rs2285479, rs2285475, rs2285474, rs2285473, rs2285472, rs2285471, rs2285469, rs876660, rs9907420, rs1049807, rs17843022, rs183898893, rs145872131, rs139515721, rs2158460, rs3803752, rs17822735, rs17222523, rs34818467, rs12946397, rs3809724, rs35476409 (same as rs397978887), rs3744214, rs3744807, rs181969864, rs4789773, rs28561791, rs28365945, rs28365946, rs2242273, rs2170297, rs11081410, rs7233770, rs7235093, rs4798691, rs7236574, rs1064058, rs1064059, rs3744878, rs1050919, rs17223038, rs117801197, rs118033234, rs76914699, rs2304191, rs148018996, rs34488963, rs3745379, rs3745385, rs141221917, rs4926171, rs142921938, rs3746295, rs151309111, rs139271842, rs115266843, rs143479651, rs140566569, rs533816037, rs75935970, rs741143, rs3746013, rs3855679, rs62107869, rs1055099, rs41290128, rs76151738, rs2302948, rs2547319, rs2547318, rs146619376, rs2284385, rs2281209, rs4911402, rs369156308, rs17333103, rs34885285, rs17424474, rs17333381, rs2233882, rs2233883, rs143199162, rs6027195, rs7509972, rs150511237, rs191574093, rs138894461, rs538007588, rs79018232, rs5999924, rs5750146, rs5756223, rs3747168, rs148345118, rs2076213, and rs6006901.

Then, the above models were optimized, the optimal thresholds (cutoff) were obtained by screening, and the ROC curves were plotted (as shown in Table 9 and FIG. 3).

TABLE 9
Optimal thresholds and AUC values for the models

| Model | Cutoff | AUC | Number of SNPs |
|---|---|---|---|
| Model 1 | −8.75 | 0.54 | 1 |
| Model 2 | −10 | 0.68 | 17 |
| Model 3 | −9.75 | 0.72 | 114 |
| Model 4 | −9.75 | 0.77 | 367 |

### Example 7: SNP Sites Related to Cylinder of the Right Eye

The data were analyzed according to the general method:

### 1. Selection of SNPs:

The genome-wide association of a total of 89,095 common variants in 8961 high myopia samples with a quantitative trait of cylinder of the right eye (rcyl) was obtained by using SNPTEST.

SNPs were screened by 4 thresholds for modeling: log 10(BF)>3; log 10(BF)>2; log 10(BF)>1.5; and log 10(BF)>1.

### 2. Acquisition of Original Genotype Profile for SNPs:

The corresponding required SNPs in vcf were extracted by using python, an additive model (with no mutation marked as 0, a mutation with one allele marked as 1, and mutation with two alleles marked as 2) was selected, and an original genotype profile for SNPs was obtained.

### 3. Imputation of Genotype Profile for SNPs:

The number of missing data of SNPs in each of the samples in the original genotype profile for SNPs was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing SNP at all; the hidden layer is provided with 20 nodes; the output layer is provided with 3 nodes; the learning rate is set to 0.003 and the number of training iterations is set to 20.

4. Classification model: Logistic Regression
1) According to the quantitative trait of cylinder of the right eye (rcyl), different thresholds were taken for modeling, AUC values were investigated, and a threshold of a model with a maximum AUC was taken as a threshold of a final classification model.

The rules are as follows:
\textcircled{1} all values of cylinder of the right eye (rcyl) are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed, and an average of 10 results was taken as a model result to ensure the objectivity of the model. The cutoff values and AUC values for the models are shown in Table 10.

TABLE 10
Cutoff values and AUC values for the models

| | Cylinder of | Cylinder of | Cylinder of | Cylinder of |
|---|---|---|---|---|
| | the right eye | the right eye | the right eye | the right eye |
| Threshold | (logbf > 3) | (logbf > 2) | (logbf > 1.5) | (logbf > 1) |
| 0 | 0.550608 | 0.570974 | 0.59237 | 0.61193 |
| −0.25 | 0.546957 | 0.567034 | 0.59853 | 0.623281 |
| −0.5 | 0.540542 | 0.557169 | 0.59293 | 0.63232 |
| −0.75 | 0.534297 | 0.572647 | 0.609598 | 0.655009 |
| −1 | 0.529161 | 0.581036 | 0.614062 | 0.659986 |
| −1.25 | 0.535496 | 0.580745 | 0.612061 | 0.659981 |
| −1.5 | 0.534695 | 0.579996 | 0.618991 | 0.68356 |
| −1.75 | 0.538655 | 0.592181 | 0.634849 | 0.697418 |
| −2 | 0.548714 | 0.596445 | 0.644839 | 0.701967 |
| −2.25 | 0.553755 | 0.604701 | 0.651634 | 0.704348 |
| −2.5 | 0.563094 | 0.620675 | 0.67254 | 0.719838 |
| −2.75 | 0.5597 | 0.624849 | 0.681968 | 0.722154 |
| −3 | 0.559078 | 0.620989 | 0.68545 | 0.732237 |
| −3.25 | 0.556586 | 0.633812 | 0.697841 | 0.741197 |
| −3.5 | 0.558745 | 0.660339 | 0.717539 | 0.742354 |
| −3.75 | 0.55906 | 0.665314 | 0.720921 | 0.757608 |
| −4 | 0.557727 | 0.658464 | 0.684801 | 0.755813 |
| −4.25 | 0.577369 | 0.668201 | 0.711378 | 0.772557 |

In the above table, the SNP combination corresponding to Model 1 (log bf>3) is:

rs42688, rs143580489, rs10484690, and rs189212243;

In the above table, the SNP combination corresponding to Model 2 (log bf>2) is:

rs12408385, rs2275365, rs374309842, rs77901289, rs147707514, rs143100530, rs41298993, rs26173, rs26098, rs1353251, rs5867289, rs1494555, rs42688, rs32210,

rs143580489, rs1204114, rs10484690, rs118151455, rs201920957, rs189212243, rs117569740, rs2271001, rs185257411, rs698028, rs4923849, rs12904657, rs62037369, rs13337155, rs17223038, rs199667693, and rs148965154.

In the above table, the SNP combination corresponding to Model 3 (log bf>1.5) is:

rs12408385, rs2275365, rs2275360, rs368345074, rs80002732, rs374309842, rs77901289, rs191506876, rs147707514, rs3087649, rs10177030, rs143100530, rs1434087, rs3748962, rs28900693, rs1077827, rs80341144, rs3213930, rs41298993, rs3749584, rs10517195, rs2271924, rs13147012, rs79482316, rs3827497, rs26173, rs26098, rs26107, rs26086, rs1353251, rs5867289, rs1494555, rs42688, rs32210, rs154483, rs7764126, rs13205210, rs143580489, rs1204114, rs10484690, rs118151455, rs200111157, rs113281844, rs201920957, rs79960636, rs112596877, rs117509972, rs189212243, rs74699763, rs2584, rs7023474, rs117569740, rs147367410, rs61748723, rs17849182, rs79735301, rs145978705, rs304494, rs115526095, rs7907476, rs4880361, rs2271001, rs111885243, rs2074233, rs622082, rs546382, rs185257411, rs145509540, rs144650014, rs17103265 (same as rs397844079), rs11570779, rs17793018, rs76846944, rs698028, rs4923849, rs12904657, rs72765429, rs111980103, rs61737565, rs4788099, rs7198606, rs62037369, rs62037371, rs10499, rs13337155, rs61742982, rs9303742, rs17223038, rs199667693, rs148965154, rs706764, rs56408444, rs117753855, rs78472358, rs6037509, rs150212573, rs147911723, and rs470101.

In the above table, the SNP combination corresponding to Model 4 (log bf>1) is: rs3795262, rs12409277, rs61735125, rs61746709, rs191051279, rs151112364, rs2291804, rs12408385, rs2275365, rs2275360, rs146696696, rs11542470, rs12386, rs74622002, rs368345074, rs142732858, rs80002732, rs374309842, rs144649977, rs78780269, rs2275863, rs77901289, rs191506876, rs117369152, rs147707514, rs6715296, rs3087649, rs7584256, rs10177030, rs11694145, rs4149520, rs143100530, rs1434087, rs77497147, rs2004888, rs11568351, rs141302000, rs3748962, rs11574750, rs16858808, rs16858811, rs141559295, rs3769838, rs6734083, rs28900693, rs4663795, rs34401965, rs76999397, rs11919775, rs1869852, rs1869851, rs1077827, rs80341144, rs3213930, rs2029618, rs75318799, rs11719458, rs33988592, rs79845192, rs41298993, rs2124541, rs66748594, rs28372853, rs572169, rs2246901, rs73189480, rs3733217, rs34611001, rs61746000, rs117486807, rs147434354, rs35762546, rs3749584, rs2271036, rs2271037, rs10517195, rs41498446, rs3733324, rs2271924, rs1801612, rs2280099, rs8582, rs13147012, rs79482316, rs3827497, rs26173, rs26098, rs26107, rs26086, rs1051489, rs2270915, rs3813474, rs1353252, rs1353251, rs1494561, rs1353250, rs5867289, rs1494558, rs1494555, rs987106, rs2233216, rs2233213, rs3217552 (same as rs397840383), rs16874505, rs3749684, rs3729668, rs28388868, rs144533176, rs42688, rs32210, rs13180243, rs154483, rs367805, rs2631368, rs117231680, rs10053292, rs7701410, rs11249552, rs142883877, rs150856424, rs3734391, rs9370867, rs77229316, rs17194565, rs117487649, rs1264319, rs35756329, rs17201248, rs190135313, rs7764126, rs11755393, rs13205210, rs3734264, rs1051115, rs74609186, rs6928630, rs143580489, rs1204114, rs10484690, rs118151455, rs143904280, rs9459355, rs9459356, rs113806172, rs10227750, rs200111157, rs539973087, rs182692351, rs13306387, rs79809732, rs113281844, rs201920957, rs236652, rs236655, rs236666, rs79960636, rs112596877, rs117509972, rs772373052, rs801841, rs1062394, rs200333762, rs4301376, rs775410128, rs3987896, rs146160185, rs17171113, rs2788473, rs189212243, rs74699763, rs2584, rs41488144, rs13306550, rs77061383, rs145349678, rs371193886, rs8178175, rs4925828, rs7023474, rs3802392, rs4879781, rs35066989, rs112858544, rs1164863576, rs16909677, rs117738198, rs117569740, rs147367410, rs61748723, rs16916100, rs2297530, rs77309503, rs77564189, rs371047734, rs17849182, rs79735301, rs145978705, rs118012211, rs76191089, rs142547455, rs2073878, rs78774780, rs140120230, rs150198392, rs10600924, rs7921838, rs3829125, rs183963660, rs17134592, rs569927230, rs41382649, rs11255267, rs144121793, rs12773592, rs139646798, rs7095891, rs11003124, rs202046718, rs7076156, rs79298145, rs3758464, rs16935560, rs304494, rs304439, rs116994405, rs11553577, rs61731067, rs7100644, rs115526095, rs7907476, rs2901895, rs7912552, rs11017735, rs4880361, rs3764896, rs1056819, rs729662, rs148238084, rs138312423, rs2271001, rs111885243, rs6485702, rs326222, rs2013867, rs901746, rs2167079, rs7120118, rs1050922, rs2074233, rs77749149, rs78038516, rs11231726, rs12146487, rs182061168, rs514076, rs145270731, rs3741172, rs1551886, rs17856219, rs1551888, rs622082, rs546382, rs488792, rs568862, rs187530720, rs2276278, rs2846412, rs143351376, rs117176605, rs10844777, rs1948516, rs2232566, rs7132546, rs12313730, rs186809868, rs35988455 (same as rs67266718), rs2178685, rs73218270, rs11547907, rs9658501, rs3914, rs555404, rs10773127, rs185257411, rs145509540, rs9315569, rs953958, rs953957, rs2183207, rs4578496, rs4614565, rs151310875, rs377238277, rs144650014, rs2274827, rs17103265 (same as rs397844079), rs11570779, rs12883882, rs17793018, rs76846944, rs2297298, rs698028, rs944050, rs2069590, rs780162072, rs73370219, rs4923849, rs12904657, rs3068, rs1046657, rs12324091, rs3743267, rs12438118, rs556233670, rs149146765, rs193114293, rs2227933, rs3214113 (same as rs397897626 or rs886038220), rs72765429, rs34855511, rs400037, rs387467, rs111980103, rs367960426, rs182015467, rs117824484, rs373109045, rs41303763, rs149271, rs180743, rs180744, rs151174, rs181206, rs61737565, rs4788099, rs7198606, rs62037369, rs62037371, rs9931989, rs10499, rs140411458 (same as rs35784618), rs71220135, rs13337155, rs141238615, rs1055708, rs114991387, rs145160385, rs4796407, rs77805790, rs756223, rs61742982, rs117908897, rs9890678, rs11080105, rs3744374, rs56135703, rs117696751, rs2164449, rs150548254, rs7216513, rs148008327, rs9303742, rs148172329, rs148241618, rs12954944, rs8092674, rs146099416, rs8095031, rs2298787, rs17069506, rs17223038, rs968688, rs199667693, rs148965154, rs8102258, rs8112094, rs79579962, rs706764, rs56408444, rs4142943, rs373547599, rs28399491, rs147818341 (same as rs368932391), rs16982743, rs60467951, rs117753855, rs78472358, rs9636153, rs6041749, rs6037509, rs6084431, rs7262903, rs2424943, rs2424944, rs73264111, rs41305787, rs45585744, rs17310782, rs2425007, rs3053708, rs10536288, rs61501849, rs2425014, rs117729108, rs11481073 (same as rs397757541), rs1998233, rs1000411, rs33969356, rs2233884, rs2072974, rs2273086, rs3746619, rs3827103, rs35295928 (same as rs886056870), rs150212573, rs147911723, rs9636833, rs138340460, rs5992854, rs201400555, rs4820658, rs11090413, and rs470101.

Then, the above models were optimized, the optimal thresholds (cutoff) were obtained by screening, and the ROC curves were plotted (as shown in Table 11 and FIG. 3).

TABLE 11
Optimal thresholds and AUC values for the models

| Model | Cutoff | AUC | Number of SNPs |
|---|---|---|---|
| Model 1 | −2.5 | 0.56 | 4 |
| Model 2 | −3.75 | 0.68 | 31 |
| Model 3 | −3.75 | 0.73 | 98 |
| Model 4 | −4.25 | 0.77 | 423 |

### Example 8: SNP Sites Related to Spherical Equivalent of the Left Eye

The data were analyzed according to the general method:

1. Selection of snps:

The genome-wide association of a total of 89,095 common variants in 8961 high myopia samples with a quantitative trait of spherical equivalent of the left eye was obtained by using SNPTEST.

snps were screened by 4 thresholds for modeling: log 10(BF)>3; log 10(BF)>2; log 10(BF)>1.5; and log 10(BF)>1, resulting in a total of 347 snps.

2. Acquisition of Original Genotype Profile for snps:

The corresponding required snps (a total of 347 snps) in vcf were extracted by using python, an additive model (with no mutation marked as 0, a mutation with one allele marked as 1, and mutation with two alleles marked as 2) was selected, and an original genotype profile for snps was obtained.

3. Imputation of Genotype Profile for snps:

The number of missing data of snps in each of the samples in the original genotype profile for snps was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing snp at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of spherical equivalent of the left eye, different thresholds were taken for modeling, AUC values were investigated, and a threshold of a model with a maximum AUC was taken as a threshold of a final classification model.

The rules are as follows:
\textcircled{1} all values of spherical equivalent of the left eye are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed, and an average of 10 results was taken as a model result to ensure the objectivity of the model.

The cutoff values and AUC values for the models are as follows:

TABLE 12
Changes in AUC at different cutoff values for each model

| | Spherical | Spherical | Spherical |
|---|---|---|---|
| | equivalent of | equivalent of | equivalent of |
| | the left eye | the left eye | the left eye |
| Threshold | (logbf > 2) | (logbf > 1.5) | (logbf > 1) |
| −2.375 | 0.550799 | 0.635073 | 0.679473 |
| −2.5 | 0.540878 | 0.627113 | 0.680991 |
| −2.625 | 0.533092 | 0.636691 | 0.701359 |
| −2.75 | 0.534028 | 0.6453 | 0.711576 |
| −2.875 | 0.533802 | 0.626549 | 0.705027 |
| −3 | 0.53394 | 0.634961 | 0.715558 |
| −3.125 | 0.539641 | 0.639814 | 0.71306 |
| −3.25 | 0.566078 | 0.640215 | 0.705086 |
| −3.375 | 0.571144 | 0.647311 | 0.714838 |
| −3.5 | 0.571909 | 0.634824 | 0.710553 |
| −3.625 | 0.566075 | 0.627383 | 0.709534 |
| −3.75 | 0.569126 | 0.639187 | 0.716408 |
| −3.875 | 0.562557 | 0.629693 | 0.689856 |
| −4 | 0.570569 | 0.63447 | 0.682237 |
| −4.125 | 0.563442 | 0.631632 | 0.683969 |
| −4.25 | 0.56921 | 0.630002 | 0.688508 |
| −4.375 | 0.567715 | 0.61881 | 0.681997 |
| −4.5 | 0.568677 | 0.628723 | 0.683786 |
| −4.625 | 0.558554 | 0.626761 | 0.683871 |
| −4.75 | 0.567522 | 0.630648 | 0.689031 |
| −4.875 | 0.561738 | 0.624201 | 0.669779 |
| −5 | 0.560125 | 0.617929 | 0.670616 |
| −5.125 | 0.549603 | 0.607757 | 0.667836 |
| −5.25 | 0.543713 | 0.612261 | 0.66813 |
| −5.375 | 0.535628 | 0.610658 | 0.660458 |
| −5.5 | 0.542911 | 0.605633 | 0.652285 |
| −5.625 | 0.544546 | 0.602471 | 0.649491 |
| −5.75 | 0.545059 | 0.59458 | 0.641174 |
| −5.875 | 0.548064 | 0.589135 | 0.642297 |
| −6 | 0.552898 | 0.587032 | 0.637445 |
| −6.125 | 0.544316 | 0.5788 | 0.623939 |
| −6.25 | 0.54324 | 0.57536 | 0.614805 |
| −6.375 | 0.540418 | 0.574967 | 0.623579 |
| −6.5 | 0.543445 | 0.57457 | 0.624312 |
| −6.625 | 0.547941 | 0.580564 | 0.626422 |
| −6.75 | 0.553386 | 0.590988 | 0.63309 |
| −6.875 | 0.552989 | 0.592758 | 0.633935 |
| −7 | 0.555648 | 0.594002 | 0.63634 |
| −7.125 | 0.552308 | 0.595995 | 0.642131 |
| −7.25 | 0.549533 | 0.597771 | 0.643079 |
| −7.375 | 0.549977 | 0.595904 | 0.643188 |
| −7.5 | 0.552348 | 0.603178 | 0.65511 |
| −7.625 | 0.555864 | 0.608289 | 0.660843 |
| −7.75 | 0.559451 | 0.610258 | 0.661631 |
| −7.875 | 0.567022 | 0.612222 | 0.66161 |
| −8 | 0.568724 | 0.617058 | 0.664973 |
| −8.125 | 0.568313 | 0.613454 | 0.664969 |
| −8.25 | 0.567342 | 0.616226 | 0.670643 |
| −8.375 | 0.57882 | 0.625066 | 0.682561 |
| −8.5 | 0.581449 | 0.625378 | 0.680737 |
| −8.625 | 0.585664 | 0.6309 | 0.692029 |
| −8.75 | 0.579653 | 0.63035 | 0.686665 |
| −8.875 | 0.581242 | 0.639301 | 0.69675 |
| −9 | 0.580508 | 0.631352 | 0.694678 |
| −9.125 | 0.585256 | 0.631297 | 0.704088 |
| −9.25 | 0.587037 | 0.633754 | 0.70209 |
| −9.375 | 0.591185 | 0.635868 | 0.705503 |
| −9.5 | 0.586098 | 0.637139 | 0.710033 |
| −9.625 | 0.578966 | 0.629522 | 0.702503 |
| −9.75 | 0.589658 | 0.631411 | 0.69907 |
| −9.875 | 0.591619 | 0.612279 | 0.707647 |
| −10 | 0.586262 | 0.601809 | 0.702544 |
| −10.125 | 0.590357 | 0.616894 | 0.727856 |
| −10.25 | 0.600563 | 0.625307 | 0.728541 |
| −10.375 | 0.594014 | 0.623387 | 0.715277 |
| −10.5 | 0.607769 | 0.637644 | 0.709326 |
| −10.625 | 0.60549 | 0.6592 | 0.725803 |
| −10.75 | 0.611865 | 0.673383 | 0.726189 |
| −10.875 | 0.604902 | 0.669434 | 0.770029 |
| −11 | 0.620724 | 0.678176 | 0.752251 |
| −11.125 | 0.615813 | 0.673593 | 0.742882 |
| −11.25 | 0.608709 | 0.680475 | 0.75357 |

The following 14 SNPs were obtained by screening with log bf>2 as criteria:

rs146160422, rs75054661, rs4959012, rs757262, rs757259, rs145293869, rs34649612, rs7157977, rs563363438, rs2277533, rs7258847, rs78661674, rs5845912, and rs8137790;

The following 76 SNPs were obtained by screening with log bf>1.5 as criteria:

rs146160422, rs1051097, rs12129653, rs17553619, rs2275856, rs150943357, rs117712357, rs192688643, rs75054661, rs2292661, rs186309103, rs11556167, rs9790356, rs9790628, rs7717970, rs12519352, rs11133847, rs2304068, rs4959012, rs757262, rs757259, rs929156, rs236444, rs17138865, rs17545405, rs148474510, rs8178175, rs55991828, rs12549018, rs529268660, rs145293869, rs1627784, rs200536689, rs573212, rs4745933, rs3751031, rs12811832, rs11168338, rs2306436, rs3741990, rs34649612, rs7157977, rs17646457, rs563363438, rs2277533, rs2452524, rs4904, rs77707876, rs370627229, rs9929443, rs149149128, rs144944402, rs113276760, rs17682036, rs59176398, rs732109, rs11260004, rs11882197, rs13535, rs2241356, rs57747888, rs397764300_rs58960485, rs16015, rs151309111, rs77561511, rs78422130, rs7258847, rs78661674, rs2183573, rs9981301, rs4624474, rs369859899, rs5845912, rs9628315, rs8138121, and rs8137790;

The following 347 SNPs were obtained by screening with log bf>1 as criteria:

rs28566954, rs377223831, rs41274480, rs146160422, rs12049046, rs16830693, rs1051097, rs12129487, rs111686948, rs12129653, rs7527057, rs144990398, rs17553619, rs140141416, rs17024525, rs117354680, rs3747626, rs16861256, rs7542316, rs7531786, rs2231241, rs2231240, rs10911390, rs10911391, rs10911392, rs12045762, rs16861394, rs6661715, rs13374714, rs13375853, rs2275856, rs6676149, rs3754234, rs2273584, rs6696123, rs11583387, rs3768066, rs3820553, rs1063128, rs3738521, rs3890478, rs28383065, rs6720415, rs6546511, rs150943357, rs77408734, rs117712357, rs3821036, rs192688643, rs6734083, rs68104327, rs35996697, rs151056400, rs116828761, rs28538922, rs138467334, rs2305613, rs115586700, rs76657333, rs79162067, rs75054661, rs4387990, rs117336796, rs77786095, rs2292661, rs12630803, rs3774765, rs1800766, rs56106326, rs139051035, rs7696197, rs80050773, rs10019009, rs61732380, rs17029087, rs3817083, rs10026790, rs11944752, rs3816873, rs2718684, rs12648093, rs186309103, rs11556167, rs9790356, rs9790628, rs4956981, rs13171103, rs4956987, rs7717970, rs12519352, rs11133847, rs117918201, rs56349002, rs2277042, rs6875303, rs6860528, rs25782, rs117977823, rs11959659, rs2304068, rs192194, rs147518598, rs74403861, rs3734056, rs139635794, rs1611212, rs28400887, rs9261424, rs4959012, rs757262, rs757259, rs929156, rs763046, rs236444, rs73733155, rs2100375, rs1051429, rs17181352, rs8191890, rs61236605, rs142944427, rs3735384, rs17138865, rs17545405, rs140518317, rs2214098, rs148474510, rs555905346, rs6945709, rs10240503, rs2074690, rs374377569, rs200506987, rs6996950, rs145988500, rs6558166, rs149587418, rs8178175, rs55991828, rs117571873, rs77428666, rs12549018, rs529268660, rs1815549, rs139669465, rs145293869, rs1627784, rs139212809, rs200536689, rs77023914, rs573212, rs28369760, rs185006838, rs75361513, rs139337135, rs75911175, rs17148873, rs35535797, rs28434439, rs34630110, rs199902798, rs34296355, rs4745933, rs906220, rs71479686, rs7069048, rs7075840, rs115337638, rs188054898, rs146804890, rs36088476, rs4757987, rs41282940, rs2986407, rs7808, rs3751031, rs201491055, rs145854081, rs202199158, rs117855259, rs117414825, rs75079562, rs1042833, rs17487041, rs12228215, rs12811832, rs11168338, rs1799986, rs2306436, rs17015438, rs45502896, rs34623199, rs140205595, rs11082, rs2305901, rs61941459, rs3741990, rs34649612, rs145055159, rs117478978, rs11551723, rs200011804, rs150666338, rs147350400, rs199627749, rs7157977, rs118004333, rs79020288, rs17646457, rs77215854, rs76611205, rs183718030, rs12915554, rs563363438, rs2277533, rs2452524, rs16963739, rs72738980, rs78309240, rs4904, rs77707876, rs3748380, rs200336355, rs3730278, rs2745136, rs2738893, rs1054645, rs527843401, rs145737959, rs2447927, rs370627229, rs2301770, rs1059676, rs9929443, rs41303763, rs149149128, rs144944402, rs2271047, rs11859725, rs113276760, rs372478569_rs71380707, rs71395334, rs11556634, rs11556635, rs3027234, rs17682036, rs144911043, rs16971620, rs41283411, rs4986763, rs4986764, rs4986765, rs540183826, rs16973424, rs146684394, rs712833, rs73350377, rs6728, rs181969864, rs59176398, rs732108, rs732109, rs76994448, rs75667305, rs28424873, rs28473343, rs3744937, rs368494050, rs146083008, rs11260004, rs11882197, rs2288415, rs10411624, rs13535, rs2217652, rs3826783, rs2230752, rs2241356, rs1978713, rs4804105, rs57747888, rs397764300_rs58960485, rs139817982, rs16015, rs151309111, rs2286663, rs117237657, rs3745764, rs189041504, rs55811458, rs2233156, rs148509022, rs407731, rs406968, rs388706, rs12052105, rs8108435, rs76579307, rs3745403, rs77561511, rs35336528, rs78422130, rs1043673, rs117172918, rs7258847, rs138317120, rs116904482, rs4815467, rs2073194, rs41275624, rs6057110, rs7828, rs1051421, rs6076122, rs78661674, rs6059011, rs148646155, rs5743533, rs114985890, rs80158178, rs201196272, rs8127537, rs2225425, rs2410104, rs1041439, rs2183573, rs1984748, rs9981301, rs4624474, rs73231245, rs885987, rs756661, rs5748471, rs202112368, rs369859899, rs6001008, rs6001009, rs9607524, rs17848337, rs5996087, rs73165153, rs5845912, rs9628315, rs8138121, rs8137790, rs68178377, and rs2232885.

Then, the above models were optimized, the optimal thresholds (cutoff) were obtained by screening, and the ROC curves were plotted (as shown in Table 13 and FIG. 3).

TABLE 13
Optimal thresholds and AUC values for the models

| Model | Threshold | AUC | Number of SNPs |
|---|---|---|---|
| Model 1 | −11 | 0.63 | 14 SNPs obtained by screening |
| | | | with logbf > 2 as criteria |
| Model 2 | −10.75 | 0.68 | 76 SNPs obtained by screening |
| | | | with logbf > 1.5 as criteria |
| Model 3 | −10.875 | 0.76 | 347 SNPs obtained by screening |
| | | | with logbf > 1 as criteria |

when 14 SNP sites obtained by screening with log bf>2 as criteria are used as markers, whether the spherical equivalent of the left eye of a subject is above −11 can be effectively determined;
when 76 SNP sites obtained by screening with log bf>1.5 as criteria are used as markers, whether the spherical equivalent of the left eye of a subject is above −10.75 can be effectively determined;
when 347 SNP sites obtained by screening with log bf>1 as criteria are used as markers, whether the spherical equivalent of the left eye of a subject is above −10.875 can be effectively determined.

### Example 9: SNP Sites Related to Sphere of the Left Eye

The data were analyzed according to the general method:

### 1. Selection of SNPs:

The genome-wide association of a total of 89,095 common variants in 8961 high myopia samples with a quantitative trait of sphere of the left eye (lsph) was obtained by using SNPTEST.

SNPs were screened by 3 thresholds for modeling: log 10(BF)>2; log 10(BF)>1.5; and log 10(BF)>1.

### 2. Acquisition of Original Genotype Profile for SNPs:

The corresponding required SNPs in vcf were extracted by using python, an additive model (with no mutation marked as 0, a mutation with one allele marked as 1, and mutation with two alleles marked as 2) was selected, and an original genotype profile for SNPs was obtained.

### 3. Imputation of Genotype Profile for SNPs:

The number of missing data of SNPs in each of the samples in the original genotype profile for SNPs was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing SNP at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of sphere of the left eye (lsph), different thresholds were taken for modeling, AUC values were investigated, and a threshold of a model with a maximum AUC was taken as a threshold of a final classification model.

The rules are as follows:
\textcircled{1} all values of sphere of the left eye (lsph) are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed, and an average of 10 results was taken as a model result to ensure the objectivity of the model. The cutoff values and AUC values for the models are shown in Table 14.

TABLE 14
Cutoff values and AUC values for the models

| | Sphere of | Sphere of | Sphere of |
|---|---|---|---|
| | the left eye | the left eye | the left eye |
| Threshold | (logbf > 2) | (logbf > 1.5) | (logbf > 1) |
| 0 | 0.466779 | 0.495077 | 0.503137 |
| −0.25 | 0.497755 | 0.502825 | 0.49763 |
| −0.5 | 0.498077 | 0.500196 | 0.50883 |
| −0.75 | 0.507211 | 0.500847 | 0.502064 |
| −1 | 0.507456 | 0.498249 | 0.497234 |
| −1.25 | 0.513711 | 0.500671 | 0.505971 |
| −1.5 | 0.510151 | 0.506826 | 0.500648 |
| −1.75 | 0.49605 | 0.500288 | 0.494876 |
| −2 | 0.498344 | 0.510515 | 0.501444 |
| −2.25 | 0.495365 | 0.501114 | 0.492943 |
| −2.5 | 0.484315 | 0.509201 | 0.503448 |
| −2.75 | 0.48179 | 0.500308 | 0.499573 |
| −3 | 0.463556 | 0.509107 | 0.498587 |
| −3.25 | 0.467544 | 0.538617 | 0.516999 |
| −3.5 | 0.47329 | 0.542781 | 0.506548 |
| −3.75 | 0.484997 | 0.546668 | 0.496637 |
| −4 | 0.482246 | 0.547342 | 0.494198 |
| −4.25 | 0.486031 | 0.526116 | 0.497099 |
| −4.5 | 0.447959 | 0.512127 | 0.469648 |

In the above table, the SNP combination corresponding to Model 2 (log bf>2) is:

rs12549018, rs529268660, rs3741990, rs34649612, rs2452524, rs57747888, rs77561511, rs78422130, rs2073194, rs9981301, rs4624474, rs5845912, and rs8137790;

In the above table, the SNP combination corresponding to Model 3 (log bf>1.5) is:

rs377223831, rs146160422, rs12129653, rs16861256, rs7542316, rs2231241, rs2231240, rs10911390, rs10911392, rs12045762, rs16861394, rs6661715, rs150943357, rs192688643, rs76657333, rs79162067, rs4387990, rs1981745, rs17466625, rs139051035, rs61732380, rs17029087, rs3817083, rs10026790, rs2718684, rs186309103, rs7717970, rs12519352, rs11133847, rs2304068, rs3734056, rs139635794, rs4959012, rs757262, rs757259, rs17181352, rs12549018, rs529268660, rs145293869, rs573212, rs117628421, rs139337135, rs522328, rs2228149, rs3751031, rs145854081, rs1042833, rs12228215, rs12811832, rs2305901, rs61941459, rs3741990, rs34649612, rs1060474, rs7157977, rs2277533, rs2452524, rs4904, rs2447927, rs1657074, rs1059676, rs9929443, rs113276760, rs732109, rs11260004, rs11882197, rs10411624, rs13535, rs2217652, rs2241356, rs74351326, rs4804105, rs57747888, rs397764300 (same as rs58960485), rs16015, rs2286663, rs34364867, rs117237657, rs189041504, rs77561511, rs78422130, rs7258847, rs862708, rs4815467, rs2073194, rs6076122, rs78661674, rs2183573, rs9981301, rs4624474, rs9607524, rs5845912, rs9628315, rs8137790, and rs76476219.

In the above table, the SNP combination corresponding to Model 4 (log bf>1) is:

rs28566954, rs377223831, rs41274480, rs146160422, rs16830693, rs1051097, rs12129487, rs12129653, rs12403630, rs58464768, rs148811681, rs41305862, rs17553619, rs71637410, rs16861256, rs7542316, rs7531786, rs2231241, rs2231240, rs10911390, rs10911391, rs10911392, rs12045762, rs16861394, rs6661715, rs138246957, rs182522436, rs13374714, rs13375853, rs2275856, rs6676149, rs3754234, rs2273584, rs6696123, rs11583387, rs3768066, rs3820553, rs1063128, rs3738521, rs76576389, rs150943357, rs34363861, rs117712357, rs71423514, rs76709099, rs192688643, rs151056400, rs116828761, rs28538922, rs138467334, rs115586700, rs76657333, rs143137431 (same as rs368878844), rs79162067, rs75054661, rs4387990, rs117336796, rs145158569, rs12630803, rs3774765, rs1800766, rs74737650, rs1981745, rs17466625, rs12330397, rs139051035, rs10019009, rs61732380, rs17029087, rs3817083, rs10026790, rs11944752, rs1800591, rs3816873, rs2718684, rs7677415, rs3796936, rs3828506, rs186309103, rs11556167, rs9790356, rs9790628, rs4956981, rs13171103, rs4956987, rs7717970, rs12519352, rs11133847, rs117918201, rs26675, rs1697144, rs2304068, rs147518598, rs57930479, rs140779761, rs74403861, rs3734056, rs139635794, rs10900967, rs1611212, rs28400887, rs9261424, rs4959012, rs757262, rs757259, rs929156, rs3888778, rs75541627, rs117340770, rs9468812, rs60399658, rs189316271, rs186815309, rs11966699, rs73733155, rs3800023, rs2273264, rs6933125, rs17181352, rs8191890, rs142944427, rs1802846, rs1131372, rs3807589, rs200730019, rs17138865, rs17545405, rs775410128, rs555905346, rs6945709, rs2074690, rs200506987, rs3739308, rs3735959, rs145988500, rs6558165, rs6558166, rs149587418, rs55991828, rs151304651, rs77428666, rs141149968, rs12549018, rs529268660, rs7003945, rs200070447, rs1815549, rs139669465, rs145293869, rs1627784, rs573212, rs28369760, rs185006838, rs117628421, rs75361513, rs41415046, rs139337135, rs522328, rs515182, rs482712, rs10760500, rs2228149, rs34630110, rs703075, rs9337951, rs906220, rs906221, rs145472189, rs146884162, rs188054898, rs541533723, rs552775933, rs146804890, rs2280134, rs36088476, rs4757987, rs2986407, rs7808, rs3751031, rs4645933, rs139692587, rs76958954, rs145854081, rs5744247, rs202199158, rs117855259, rs117414825, rs2291075, rs117665966, rs1042833, rs17487041, rs12228215, rs12811832, rs11168338, rs138189784, rs75577568, rs2306436, rs369302829, rs2305901, rs61941459, rs3741990, rs34649612, rs1248046, rs145055159, rs117478978, rs200011804, rs150666338, rs1060474, rs10139379, rs8008670, rs8009789, rs8022395, rs7155123, rs199627749, rs7157977, rs79020288, rs183718030, rs9972231, rs563363438, rs2277533, rs2452524, rs78309240, rs4904, rs77707876, rs1545119, rs3748380, rs200336355, rs2745136, rs60526088, rs2738893, rs1054645, rs2447927, rs1657074, rs370627229, rs2301770, rs1059676, rs9929443, rs55969568, rs71220135, rs16943483, rs11859725, rs113276760, rs5818479, rs71395334, rs78806449, rs1043592, rs17682036, rs144911043, rs16971620, rs540183826, rs16973424, rs146684394, rs80345815, rs732109, rs79712753, rs28424873, rs189184481, rs61735591, rs7251165, rs146083008, rs147472740, rs4134853, rs11260004, rs117622602, rs11882197, rs10411624, rs13535, rs2217652, rs3826783, rs2230752, rs2241356, rs74351326, rs1978713, rs4804105, rs57747888, rs397764300 (same as rs58960485), rs139817982, rs16015, rs151309111, rs2286663, rs34364867, rs117237657, rs189041504, rs1865094, rs2233156, rs2302987, rs148509022, rs188387562, rs407731, rs406968, rs388706, rs12052105, rs3745403, rs34604090, rs77561511, rs35336528, rs78422130, rs7258847, rs862708, rs2361119, rs4801478, rs4815467, rs2073194, rs6076122, rs78661674, rs11546155, rs114985890, rs2236001, rs3989369, rs8127537, rs2225425, rs2410104, rs1041439, rs2183573, rs1984748, rs9981301, rs4624474, rs1051367, rs2073733, rs885985, rs1803545, rs148650930, rs2072012, rs9607524, rs17523588, rs17848337, rs73165153, rs5845912, rs9628315, rs8137790, rs76476219, rs55861809, and rs68178377.

Then, the above models were optimized, the optimal thresholds (cutoff) were obtained by screening, and the ROC curves were plotted (as shown in Table 15 and FIG. 3).

TABLE 15
Optimal thresholds and AUC values for the models

| Model | Cutoff | AUC | Number of SNPs |
|---|---|---|---|
| Model2 | −1.25 | 0.51 | 13 |
| Model3 | −3.75 | 0.55 | 95 |
| Model4 | −3.25 | 0.51 | 333 |

### Example 10: SNP Sites Related to Cylinder of the Left Eye

The data were analyzed according to the general method:

### 1. Selection of SNPs:

The genome-wide association of a total of 89,095 common variants in 8961 high myopia samples with a quantitative trait of cylinder of the left eye (lcyl) was obtained by using SNPTEST.

SNPs were screened by 4 thresholds for modeling: log 10(BF)>3; log 10(BF)>2; log 10(BF)>1.5; and log 10(BF)>1.

### 2. Acquisition of Original Genotype Profile for SNPs:

The corresponding required SNPs in vcf were extracted by using python, an additive model (with no mutation marked as 0, a mutation with one allele marked as 1, and mutation with two alleles marked as 2) was selected, and an original genotype profile for SNPs was obtained.

### 3. Imputation of Genotype Profile for SNPs:

The number of missing data of SNPs in each of the samples in the original genotype profile for SNPs was calculated, and the corresponding sample was removed in the case of the proportion of missing data of >5% (N_missing/N_allsamples). For samples with the proportion of missing data of <5%, imputation was performed by using the artificial neural network.

The artificial neural network has three layers including an input layer, a hidden layer, and an output layer: the number of nodes in the input layer is len(label_data), that is, there is no missing SNP at all; the hidden layer is provided with 20 nodes; the output layer is provided with 3 nodes; the learning rate is set to 0.003 and the number of training iterations is set to 20.

### 4. Classification Model: Logistic Regression

1) According to the quantitative trait of cylinder of the left eye (lcyl), different thresholds were taken for modeling, AUC values were investigated, and a threshold of a model with a maximum AUC was taken as a threshold of a final classification model.

The rules are as follows:
\textcircled{1} all values of cylinder of the left eye (lcyl) are transversed and are each used as a threshold to divide samples into two groups;
\textcircled{2} the number of samples in each group should not be less than 100.
2) Samples were classified by using logistic regression with the following parameters:
LogisticRegression(C=100, class weight=‘balanced’, dual=False,
   fit_intercept=True, intercept_scaling=1, max_iter=10,
   multi_class=‘ovr’, n_jobs=1, penalty=‘11’, random_state=0,
   solver=‘liblinear’, tol=0.0001, verbose=0, warm_start=False)

Although the method is called logistic regression, it is a linear model used for classification rather than regression.

By default, the logistic regression applies regularization. This is common in machine learning, and one of its advantages is that it improves the stability of the values, and the absence of regularization amounts to setting C to a very high value.

As an optimization problem, the following cost function was minimized by using the binary class L2 penalized logistic regression:

$${min}_{w , c} \frac{1}{2} {w}^{T} w + C {\sum }_{i = 1}^{n} log ( exp ( - {y}_{i} ( {X}_{i}^{T} w + c ) ) + 1 ) .$$

Similarly, the following optimization problem was solved by using the L1 regularized logistic regression:

$${min}_{w , c} {‖ w ‖}_{1} + C {\sum }_{i = 1}^{n} log ( exp ( - {y}_{i} ( {X}_{i}^{T} w + c ) ) + 1 ) .$$

Ten random numbers from 0 to 100 were selected as random seeds by using a sklearn.model_selection function, samples were divided into 10 groups of training and verification sets, a 10-fold cross-validation was performed, and an average of 10 results was taken as a model result to ensure the objectivity of the model. The cutoff values and AUC values for the models are shown in Table 16. The diagnostic efficacy evaluation curves are shown in FIG. 3.

TABLE 16
Cutoff values and AUC values for the models

| | Cylinder of | Cylinder of | Cylinder of | Cylinder of |
|---|---|---|---|---|
| | the left eye | the left eye | the left eye | the left eye |
| Threshold | (logbf > 3) | (logbf > 2) | (logbf > 1.5) | (logbf > 1) |
| 0 | 0.49887 | 0.511357 | 0.549666 | 0.528075 |
| −0.25 | 0.528672 | 0.549829 | 0.566279 | 0.599838 |
| −0.5 | 0.535288 | 0.550686 | 0.549822 | 0.630882 |
| −0.75 | 0.530808 | 0.550681 | 0.565534 | 0.635863 |
| −1 | 0.53485 | 0.563624 | 0.573678 | 0.642584 |
| −1.25 | 0.539723 | 0.570035 | 0.586352 | 0.653799 |
| −1.5 | 0.533106 | 0.568227 | 0.591546 | 0.659202 |
| −1.75 | 0.538502 | 0.579591 | 0.605397 | 0.679366 |
| −2 | 0.535328 | 0.579958 | 0.601886 | 0.68451 |
| −2.25 | 0.542564 | 0.585359 | 0.610045 | 0.690159 |
| −2.5 | 0.552069 | 0.596582 | 0.6197 | 0.697046 |
| −2.75 | 0.55524 | 0.607506 | 0.633477 | 0.706191 |
| −3 | 0.567302 | 0.630018 | 0.648689 | 0.733765 |
| −3.25 | 0.56082 | 0.615177 | 0.641732 | 0.734157 |
| −3.5 | 0.559743 | 0.624561 | 0.634146 | 0.748583 |
| −3.75 | 0.566928 | 0.632189 | 0.633889 | 0.742512 |
| −4 | 0.559798 | 0.636081 | 0.602027 | 0.775707 |
| −4.25 | 0.564557 | 0.643387 | 0.610718 | 0.768389 |
| −4.5 | 0.562213 | 0.619267 | 0.579831 | 0.733829 |

In the above table, the SNP combination corresponding to Model 1 (log bf>3) is:

rs26173, rs26098, rs26107, rs26086, rs143580489, rs10484690, and rs117984746;

In the above table, the SNP combination corresponding to Model 2 (log bf>2) is:

rs2296474, rs2765015, rs3766165, rs145378993, rs13306137, rs147707514, rs112218972, rs3796381, rs26173, rs26098, rs26107, rs26086, rs987106, rs75126858, rs61734326, rs2278226, rs6928630, rs143580489, rs1204114, rs10484690, rs74699763, rs11574345, rs4925828, rs304494, rs304439, rs304448, rs4763975, rs377238277, rs144650014, rs12438118, rs41303763, rs9797618, rs10420552, rs4802029, rs28512414, rs1975937, rs2075284, rs3095726, rs34785154, rs60704260, rs6084431, rs147911723, rs41279352, rs2427624, and rs117984746;

In the above table, the SNP combination corresponding to Model 3 (log bf>1.5) is:

rs2296474, rs307377, rs2765015, rs3766165, rs145378993, rs61746709, rs114369626, rs4147830, rs540468026, rs80105432, rs45445497, rs13306137, rs191506876, rs147707514, rs4149520, rs4988956, rs4988957, rs10192036, rs10204137, rs4988958, rs10192157, rs10206753, rs3755276, rs112218972, rs3749022, rs76999397, rs35220123, rs80341144, rs3796381, rs117486807, rs147434354, rs76469954, rs26173, rs26098, rs26107, rs26086, rs987106, rs75126858, rs61734326, rs2278226, rs144289000, chr5-140568680, rs7701410, rs117600221, rs150856424, rs3188, rs6928630, rs143580489, rs1204114, rs10484690, rs775410128, rs13225910, rs61733329, rs189212243, rs74699763, rs11574345, rs8178175, rs10092164, rs11539113, rs68008113, rs7462883, rs66881636, rs61747644, rs2721167, rs2620644, rs2250891, rs2250657, rs2721176, rs4925828, rs117569740, rs142643091, rs140966742, rs3758464, rs16935560, rs144762118, rs7899453, rs3740252, rs304494, rs304439, rs304448, rs148238084, rs622082, rs546382, rs189385634, rs4763975, rs2232566, rs7132546, rs5742612, rs35767, rs2861550, rs377238277, rs144650014, rs35405829, rs11569599, rs10130914, rs140879339, rs12438118, rs182015467, rs41303763, rs142805492, rs8055740, rs75743672, rs75277269, rs76896732, rs2164449, rs2230601, rs9303742, rs79577494, rs9797618, rs10420552, rs4802029, rs28512414, rs1975937, rs958305, rs2075284, rs3095726, rs34785154, rs60704260, rs2034891, rs117753855, rs78472358, rs8099961, rs58634535, rs6084431, rs147911723, rs41279352, rs2427624, rs117984746, rs470101, and rs2267163;

In the above table, the SNP combination corresponding to Model 4 (log bf>1) is:

rs6690013, rs609805, rs621668, rs2274264, rs2274263, rs2296474, rs10907179, rs307349, rs307355, rs35744813, rs307377, rs307371, rs2296471, rs2765015, rs34091023 (same as rs371194568), rs1171, rs3766165, rs145378993, rs115226069, rs61746709, rs2274329, rs2274330, rs3765967, rs148750290, rs114369626, rs35533335, rs74622002, rs145619910, rs141216171, rs146227933, rs145717494, rs4147830, rs3215952, rs2229783, rs17127203, rs1676486, rs1763347, rs368345074, rs7556386, rs540468026, rs374309842, rs942963, rs77181873, rs80105432, rs33941127, rs45445497, rs12026637, rs144649977, rs78780269, rs3813972, rs60555365, rs335524, rs4149230, rs2292559, rs2292561, rs41310561, rs147519768, rs13306137, rs1800672, rs2296801, rs77082151, rs76255469, rs12135078, rs112212885, rs62121538, rs191506876, rs147707514, rs74867758, rs4665385, rs3792253, rs1126497, rs4149520, rs3771175, rs4988956, rs4988957, rs10192036, rs10204137, rs4988958, rs10192157, rs10206753, rs3755276, rs112218972, rs116177854, rs2123465, rs2116664, rs1469651, rs1469650, rs1053394, rs77698806, rs1434087, rs3749022, rs145346406, rs6734083, rs11676792, rs53576, rs76999397, rs2075310, rs35993975, rs33917318, rs17032276, rs17032278, rs17032283, rs35220123, rs9879080, rs7649243, rs34608006, rs6805869, rs9811771, rs2272125, rs2272124, rs7626117, rs7647987, rs6796538, rs35229877, rs11919713, rs1969154, rs1985428, rs3732675, rs80341144, rs181735010, rs3796381, rs76407344, rs11719458, rs2292661, rs79845192, rs74682201, rs77220512, rs35070271, rs79144888, rs76371354, rs78233713, rs117486807, rs147434354, rs6815254, rs3756247, rs1457976435, rs191674075, rs3749584, rs10517195, rs78205702, rs76469954, rs26173, rs26098, rs26107, rs26086, rs987106, rs62387135, rs79420400, rs75126858, rs61734326, rs2278226, rs2228112, rs42688, rs32210, rs6886922, rs118179072, rs144289000, chr5-140568680, rs182889312, rs7701410, rs117600221, rs142883877, rs150856424, rs3734391, rs140750807, rs138075877, rs2274347, rs3188, rs62622418, rs74609186, rs9396251, rs6928630, rs143580489, rs1204114, rs10484690, rs370140172, rs1048886, rs78873069, rs73333875, rs200555224, rs80190153, rs201920957, rs150559408, rs775410128, rs189382707, rs79848686, rs17171113, rs7791608, rs17173702, rs17173703, rs13225910, rs61733329, rs189212243, rs74699763, rs11550509, rs10866818, rs11574345, rs8178175, rs10092164, rs11539113, rs34596274, rs68008113, rs7462883, rs66881636, rs7812496, rs3739287, rs61747644, rs7840439, rs2721167, rs2620644, rs2250891, rs2250657, rs2721176, rs4925828, rs2721190, rs2721189, rs2721172, rs2281747, rs10961672, rs140349388_rs3833713, rs7023474, rs80235406, rs4879781, rs112858544, rs1164863576, rs6560142, rs879369, rs117569740, rs4838184, rs371047734, rs522328, rs515182, rs482712, rs199901947, rs12684476, rs7043416, rs113809617, rs7079747, rs142643091, rs74704054, rs140966742, rs3758464, rs16935560, rs144762118, rs7899453, rs3740252, rs2762528, rs304494, rs304439, rs304448, rs2254619, rs11017735, rs2298192, rs193087745, rs756924, rs2074022, rs739502, rs12577654, rs148238084, rs111885243, rs57477011, rs2074233, rs2067477, rs77749149, rs145270731, rs622082, rs546382, rs189385634, rs2852365, rs1057992, rs642573, rs3018301, rs12272086, rs5103, rs5069, rs377232839, rs201872688, rs4763975, rs150028933, rs2232566, rs7132546, rs2304821, rs148804891, rs5742612, rs35767, rs10778292, rs11112201, rs10847164, rs117739120, rs185257411, rs145509540, rs2861550, rs2282407, rs377238277, rs144650014, rs35405829, rs145005726, rs45585336, rs186889719, rs11569599, rs11570779, rs76846944, rs698028, rs10130914, rs1808975, rs140879339, rs9919918, rs11853872, rs25397, rs3075142_rs397774483, rs12904657, rs12438118, rs7170185, rs17273206, rs3815003, rs2227933, rs74839044, rs34855511, rs577645079, rs76468382, rs117474153, rs367960426, rs182015467, rs41303763, rs187940556, rs13337155, rs138428580, rs1055708, rs62054627, rs1048149, rs62052673, rs142805492, rs8049480, rs758803114, rs8055740, rs138169571, rs7222126, rs11657498, rs200205820, rs4796407, rs2302763, rs138625739, rs201373267, rs2302160, rs34747500, rs56135703, rs28691051, rs200996148, rs75743672, rs729782, rs117696751, rs75277269, rs76896732, rs2164449, rs2230601, rs9303742, rs35114461, rs17223038, rs148965154, rs79577494, rs79579962, rs1054533, rs9797618, rs10420552, rs4802029, rs28512414, rs1975937, rs958305, rs2075284, rs3095726, rs34785154, rs60704260, rs112805435, rs188724242, rs2034891, rs117753855, rs112910446, rs8100225, rs1657535, rs78472358, rs8099961, rs142900000, rs8958, rs58634535, rs6084431, rs2280091, rs2280092, rs3746642, rs235768, rs235769, rs221984, rs2424943, rs2424944, rs73264111, rs41305787, rs45585744, rs2295096, rs2071971, rs117358971, rs112959405, rs1124178, rs3746619, rs3827103, rs35295928_rs886056870, rs150212573, rs147911723, rs41279352, rs2427624, rs139729932, rs117984746, rs2276254, rs141212516 (same as rs377432980 or rs386365935 or rs386394882), rs7283989, rs11701124, rs8131313, rs35796750, rs143527452, rs45604134, rs1052756, rs1052773, rs2240111, rs165815, rs2073747, rs5762430, rs470101, rs2267163, rs5997714, rs117291362, and rs11553143.

Then, the above models were optimized, the optimal thresholds (cutoff) were obtained by screening, and the ROC curves were plotted (as shown in Table 17 and FIG. 3).

TABLE 17
Optimal thresholds and AUC values for the models

| Model | Cutoff | AUC | Number of SNPs |
|---|---|---|---|
| Model1 | −2.5 | 0.56 | 7 |
| Model2 | −3.75 | 0.68 | 45 |
| Model3 | −3.75 | 0.73 | 130 |
| Model4 | −4.25 | 0.77 | 429 |

What is claimed is:

1. A method for preventing or treating a vision problem, comprising:
step 1, detecting biological samples from subjects to obtain genotypes of an SNP site combination;
step 2, inputting results obtained by the detection in step 1 into a myopia model for processing, classifying the subjects according to the genotypes of the SNP site combination, and selecting those susceptible to a vision problem by screening; and,
step 3, administering corrective lenses or administering a pharmaceutically effective amount of a therapeutic agent to those susceptible to a vision problem selected by the classification in step 2; wherein,
the SNP site combination is selected from:
a) an SNP site combination related to myopia or high myopia, comprising: one or more of rs10484690, rs117984746, rs1196455, rs143477571, rs143580489, rs189212243, rs2296651, rs2303221, rs2547319, rs26173, rs3817585, rs41303763, rs42688, rs61746299, rs78587889, and rs80094829;
b) an SNP site combination related to spherical equivalent difference between the two eyes;
c) an SNP site combination related to spherical equivalent of the worst eye;
d) an SNP site combination related to spherical equivalent of the right eye;
e) an SNP site combination related to sphere of the right eye;
f) an SNP site combination related to cylinder of the right eye;
g) an SNP site combination related to spherical equivalent of the left eye;
h) an SNP site combination related to sphere of the left eye; and
i) an SNP site combination related to cylinder of the left eye;
the SNP site combination related to spherical equivalent difference between the two eyes comprises a first SNP site combination related to spherical equivalent difference between the two eyes, a second SNP site combination related to spherical equivalent difference between the two eyes, a third SNP site combination related to spherical equivalent difference between the two eyes, and a fourth SNP site combination related to spherical equivalent difference between the two eyes;
the SNP site combination related to spherical equivalent of the worst eye comprises a first SNP site combination related to spherical equivalent of the worst eye, a second SNP site combination related to spherical equivalent of the worst eye, a third SNP site combination related to spherical equivalent of the worst eye, and a fourth SNP site combination related to spherical equivalent of the worst eye;
the SNP site combination related to spherical equivalent of the right eye comprises a first SNP site combination related to spherical equivalent of the right eye, a second SNP site combination related to spherical equivalent of the right eye, and a third SNP site combination related to spherical equivalent of the right eye;
the SNP site combination related to sphere of the right eye comprises a first SNP site combination related to sphere of the right eye, a second SNP site combination related to sphere of the right eye, and a third SNP site combination related to sphere of the right eye;
the SNP site combination related to cylinder of the right eye comprises a first SNP site combination related to cylinder of the right eye, a second SNP site combination related to cylinder of the right eye, a third SNP site combination related to cylinder of the right eye, and a fourth SNP site combination related to cylinder of the right eye;
the SNP site combination related to spherical equivalent of the left eye comprises a first SNP site combination related to spherical equivalent of the left eye, a second SNP site combination related to spherical equivalent of the left eye, and a third SNP site combination related to spherical equivalent of the left eye;
the SNP site combination related to sphere of the left eye comprises a first SNP site combination related to sphere of the left eye, a second SNP site combination related to sphere of the left eye, and a third SNP site combination related to sphere of the left eye;
the SNP site combination related to cylinder of the left eye comprises a first SNP site combination related to cylinder of the left eye, a second SNP site combination related to cylinder of the left eye, a third SNP site combination related to cylinder of the left eye, and a fourth SNP site combination related to cylinder of the left eye;
the first SNP site combination related to spherical equivalent difference between the two eyes comprises: one or more of rs80094829, rs1196455, rs61746299, rs3817585, and rs2296651;
the second SNP site combination related to spherical equivalent difference between the two eyes comprises: one or more of rs138802237, rs80094829, rs1196455, rs61746299, rs17846715, rs77819192, rs74711761, rs186128635, rs3817585, rs166508, rs11745612, rs164074, rs164075, rs164076, rs140779761, rs17652080, rs17708515, rs17110944, rs140691873, rs1042454, rs1613776, rs57344881, rs2296651, rs2302230, rs2302231, rs4147910, rs74406853, rs199946960, rs35689937, rs34378744, rs71357953, rs71357954, rs201326575, and rs2075937;
the third SNP site combination related to spherical equivalent difference between the two eyes comprises: one or more of rs183232121, rs138802237, rs7539625, rs80094829, rs1196455, rs61746299, rs150026164, rs12043009, rs17846715, rs4653415, rs77819192, rs117238208, rs34117315, rs4851287, rs74711761, rs13422424, rs186128635, rs150129946, rs35678, rs2289272, rs3817585, rs166508, rs73135528, rs73135529, rs73135530, rs73135535, rs73135545, rs36077176, rs73135584, rs137919012, rs3813832, rs11745612, rs56969869, rs397741115_rs56191507, rs16899974, rs164073, rs164074, rs164075, rs164076, rs140779761, rs17652080, rs17708515, rs17110944, rs6627, rs61748571, rs137899339, rs117192504, rs1109208, rs73689406, rs192085105, rs1830035, rs1830036, rs17138865, rs17545405, rs140691873, rs17553698, rs3810925, rs111748634, rs111841972, rs4073310, rs2271460, rs1042454, rs1044476, rs2274351, rs1613776, rs57344881, rs76274604, rs694066, rs7123133, rs7123272, rs28734900, rs12797894, rs17102543, rs1050395, rs181464388, rs2296651, rs3818188, rs1455773, rs13380006, rs199816463, rs429744, rs370627229, rs3743600, rs138793855, rs35896669, rs2302230, rs2302231, rs2907373, rs116067668, rs138221111, rs3752229, rs3764648, rs4147910, rs4147914, rs80054150, rs150907742, rs200222185, rs74406853, rs199946960, rs35689937, rs34378744, rs6511379, rs71357953, rs71357954, rs28678477, rs201326575, rs874100, rs1534879, rs2075937, and rs2239749;
the fourth SNP site combination related to spherical equivalent difference between the two eyes comprises: one or more of rs12133253, rs1573753, rs7512414, rs34109355, rs34976233, rs35013594, rs7528572, rs1541185, rs146160422, rs41303641, rs41311274, rs183232121, rs75321043, rs79777494, rs138802237, rs7664, rs1884444, rs7539625, rs76475726, rs599839, rs12083584, rs374505350, rs80094829, rs1196455, rs61746299, rs200519781, rs150026164, rs12043009, rs1375211271_rs373796693, rs17846707, rs17846715, rs3747626, rs1301522897_rs397775888, rs17846717, rs17846718, rs41288127, rs12087593, rs17380127, rs17380141, rs77202104, rs4653415, rs77819192, rs117238208, rs28383065, rs3731572, rs4148186, rs151242484, rs34117315, rs370613331, rs4851287, rs144642601, rs200736867, rs74711761, rs13422424, rs185464655, rs148663631, rs186128635, rs79028939, rs150129946, rs236843, rs10173558, rs150979437, rs140446783, rs56847463, rs183596892, rs117354149, rs35678, rs2075354, rs2289272, rs3817585, rs166508, rs370554533, rs78946357, rs73135513, rs73135528, rs73135529, rs73135530, rs73135535, rs73135545, rs36077176, rs73135584, rs117279588, rs137919012, rs17552950, rs151025946, rs77422573, rs3813832, rs4752, rs77676024, rs7687613, rs74991481, rs77885682, rs13152799, rs2276979, rs11745612, rs113222960, rs56969869, rs12523335, rs397741115_rs56191507, rs16899974, rs2560306, rs186414752, rs351260, rs164073, rs164074, rs164075, rs164076, rs140779761, rs17652080, rs17708515, rs17110944, rs111995800, rs3839291_rs398000092, rs6627, rs17136239, rs61748571, rs2815822, rs35374726, rs77229316, rs3094225, rs2281831, rs113689068, rs137899339, rs17710008, rs7763726, rs117192504, rs186624179, rs2292498, rs3763384, rs146123998, rs17779352, rs192189247, rs1109208, rs73689406, rs192085105, rs1830035, rs1830036, rs17138865, rs17545405, rs41350445, rs34587586, rs78637678, rs4503083, rs188235566, rs140691873, rs79930604, rs41297187, rs41307479, rs17553698, rs3810925, rs111748634, rs111841972, rs7850697, rs2231406, rs4073310, rs2271460, rs80042909, rs11200920, rs11200933, rs1042454, rs1934951, rs1044476, rs11191295, rs7081110, rs7081467, rs2274351, rs1613776, rs3135802, rs6693, rs183813046, rs57344881, rs907610, rs80326119, rs76274604, rs77600363, rs486584, rs12804382, rs12805133, rs201373803, rs694066, rs189385634, rs149080490, rs138619667, rs7123133, rs7123272, rs28734900, rs12797894, rs7307854, rs76066543, rs139938131, rs7980396, rs3217157_rs35118302_rs537549359, rs149247686, rs75656873, rs2277373, rs59261129, rs181052973, rs183437347, rs17102543, rs1050395, rs3751129, rs77973991, rs2036771, rs4759544, rs181464388, rs112163489, rs9594738, rs3210286, rs191708663, rs149954510, rs440466, rs2331979, rs7159367, rs34598192, rs2277475, rs3729818, rs117109392, rs58154675, rs2295106, rs2295107, rs2235967, rs7143196, rs2296651, rs116897063, rs562694521, rs536155089, rs1884541, rs3818188, rs200138830, rs7177192, rs2412541, rs11858113, rs8041534, rs8040502, rs11070285, rs2412544, rs141891738, rs12232341, rs143563919, rs3743476, rs1455773, rs13380006, rs74642800, rs199816463, rs373684171, rs429744, rs370627229, rs78671991, rs529955941, rs41303763, rs11867123, rs7206481, rs3743600, rs11656203, rs138793855, rs115332545, rs3736238, rs147085136, rs11649834, rs140947135, rs8556, rs2303015, rs3826301, rs35896669, rs75743672, rs729782, rs59589773, rs184602743, rs2302230, rs2302231, rs2907373, rs140010427, rs3744032, rs2748428, rs73410309, rs11657217, rs148660676, rs1192271300_rs397858239, rs566655, rs116067668, rs138221111, rs189222917, rs375483921, rs3752229, rs3764648, rs4147910, rs4147914, rs1610096, rs2074442, rs117187726, rs2074454, rs80054150, rs150907742, rs4807578, rs143662386, rs28480458, rs139113505, rs200222185, rs74406853, rs199946960, rs35689937, rs34378744, rs6511379, rs71357953, rs71357954, rs1801015, rs80027112, rs4635, rs56276635, rs184733141, rs79863139, rs181599488, rs138399909, rs324125, rs8109631, rs80087697, rs200619037, rs28678477, rs2273423, rs6023533, rs5743513, rs817343, rs45566234, rs7281104, rs14194, rs201326575, rs874100, rs202195284, rs1894473, rs17670808, rs17738527, rs17738540, rs9606738, rs9608944, rs9608956, rs1534879, rs2075937, rs2239749, rs202112368, rs188655025, and rs11541025;
the first SNP site combination related to spherical equivalent of the worst eye comprises: one or more of rs78587889, rs41303763, and rs2303221;
the second SNP site combination related to spherical equivalent of the worst eye comprises: one or more of rs78587889, rs188480146, rs2292661, rs6894260, rs4957836, rs148474510, rs200506987, rs145293869, rs2248077, rs2811736, rs2297722, rs2297723, rs9987876, rs7157977, rs41303763, rs2303221, rs145438063, rs151309111, rs6076122, rs2183573, rs9981301, and rs5756223;
the third SNP site combination related to spherical equivalent of the worst eye comprises: one or more of rs3103778, rs2746342, rs78587889, rs140141416, rs17024525, rs2288182, rs188480146, rs6734083, rs75054661, rs12492484, rs2292661, rs55947930, rs16837029, rs149213974, rs6894260, rs45625534, rs4957836, rs147518598, rs1042044, rs12671595, rs186526946, rs138414411, rs3802064, rs75456709, rs76427362, rs2073793, rs140518317, rs2214098, rs148474510, rs876087, rs200506987, rs8178175, rs145293869, rs200021348_rs552590290, rs16909677, rs2248077, rs2811736, rs2297722, rs2297723, rs9987876, rs34630110, rs141296329, rs117520659, rs3750996, rs3847553, rs142908193, rs1042669, rs12811832, rs78692679, rs11551723, rs7157977, rs3759779, rs3742770, rs530110551, rs3742464, rs3736911, rs11849022, rs77481241, rs3212112, rs9282879, rs55958706, rs3212056, rs114770841, rs3742368, rs563363438, rs2738893, rs1132358, rs41303763, rs2303221, rs145438063, rs151309111, rs139271842, rs533816037, rs305974, rs407731, rs406968, rs388706, rs2302948, rs2547319, rs7258847, rs4815467, rs41275624, rs6057110, rs6076122, rs4911402, rs2183573, rs9981301, rs4624474, rs5756223, and rs5845912;
the fourth SNP site combination related to spherical equivalent of the worst eye comprises: one or more of rs3795289, rs17033266, rs61785839, rs77649572, rs202140107, rs2275365, rs77275487, rs6676052, rs3103778, rs3014246, rs3014240, rs35016075_rs59669671, rs28375469, rs116861599, rs2746342, rs2457829, rs10889315, rs148811681, rs41305862, rs1410871, rs61729628, rs17553619, rs78587889, rs140141416, rs17024525, rs149097658, rs822441, rs183812847, rs112946310, rs12568050, rs12564283, rs75401237, rs10749643, rs2288182, rs400917, rs201752014, rs75526749, rs17189743, rs188480146, rs528123098, rs61748134, rs2228184, rs143686122, rs1434087, rs13002576, rs192688643, rs6734083, rs68104327, rs35996697, rs6437368, rs1077827, rs76657333, rs75054661, rs547922447, rs143654927, rs2286786, rs115250058, rs117336796, rs12492484, rs2292661, rs2276740, rs3774765, rs2245278, rs55947930, rs75144949, rs3732755, rs16837029, rs11732898, rs7696197, rs6841898, rs114264770, rs75058604, rs149213974, rs17001890, rs61732380, rs17029087, rs76996680, rs186309103, rs2303710, rs3749692, rs181303223, rs150821721, rs76381196, rs139298086_rs17839482, rs555615570, rs6894260, rs13189822, rs45625534, rs4957836, rs147518598, rs3829986, rs4608967, rs564109, rs150157174, rs2735059, rs4959012, rs757262, rs757259, rs117333063, rs185990098, rs41542812, rs11966699, rs4711571, rs4711572, rs2235868, rs1042044, rs3804502, rs2100375, rs118003870, rs75817012, rs17181352, rs35517174, rs17132399, rs2292498, rs3763384, rs12671595, rs186526946, rs138414411, rs13438494, rs3802064, rs75456709, rs76427362, rs2073793, rs140518317, rs2214098, rs79413133, rs2230156, rs148474510, rs41465145, rs876087, rs200506987, rs537284787, rs8178175, rs55991828, rs12549018, rs34961221, rs78240711, rs139669465, rs11144089, rs117169590, rs145293869, rs200021348_rs552590290, rs16909677, rs117175949, rs10481674, rs41276795, rs141549766, rs147026086, rs188197626, rs7038042, rs11849, rs2275161, rs10781513, rs2248077, rs2811736, rs2297722, rs2297723, rs9987876, rs7079747, rs34630110, rs148582275, rs4745933, rs111835023, rs185249748, rs117768118, rs141296329, rs117520659, rs45498396, rs3750996, rs183484, rs9937, rs2304733, rs147852038, rs139692587, rs10792769, rs2298573, rs4753069, rs3847553, rs2962118, rs142908193, rs1042669, rs11047401, rs12811832, rs11168338, rs2277373, rs1150057, rs1799986, rs1050395, rs78692679, rs3835190, rs201814780, rs11551723, rs7992732, rs7157977, rs10143899, rs3759779, rs7142098, rs118004333, rs3742770, rs3742768, rs3742767, rs143477571, rs530110551, rs3742464, rs3736911, rs11849022, rs77481241, rs3212112, rs9282879, rs55958706, rs3212056, rs34100926, rs114770841, rs3742368, rs4390564, rs72486624, rs7172888, rs563363438, rs12904657, rs4904, rs72546313, rs2745136, rs2738893, rs1054645, rs1132358, rs79763179, rs3743807, rs55969568, rs9941128, rs41303763, rs2303221, rs11859725, rs113276760, rs145438063, rs11550470, rs145797873, rs11556634, rs56126318, rs16954357, rs397718340_rs5819492, rs9899785, rs375644567, rs185576254, rs183898893, rs4986765, rs146684394, rs181969864, rs2242273, rs2170297, rs11081410, rs7233770, rs7235093, rs7236574, rs11659762, rs116067668, rs11538683, rs55646937, rs148018996, rs1143698, rs10412834, rs11260004, rs11882197, rs2288415, rs150461309, rs3746295, rs151309111, rs3810209, rs4808643, rs139271842, rs200222185, rs189120571, rs148003617, rs533816037, rs189041504, rs305968, rs305974, rs407731, rs406968, rs388706, rs2302948, rs182509214, rs198977, rs2547319, rs2547318, rs7258847, rs4815467, rs41275624, rs6057110, rs6076122, rs4911402, rs114985890, rs80158178, rs3746488, rs76747058, rs143199162, rs77432560, rs116846998, rs2183573, rs1984748, rs9981301, rs4624474, rs2838379, rs4566449, rs915770, rs2870984, rs2073733, rs41281587, rs3747135, rs3747136, rs3747137, rs5756223, rs3747168, rs369859899, rs6006901, rs5845912, rs9628315, and rs8137790;
the first SNP site combination related to spherical equivalent of the right eye comprises: one or more of rs10889315, rs75144949, rs149213974, rs754615, rs45583335, rs2529438, rs2072236, rs2240403, rs78155900, rs114097201, rs2811736, rs2297722, rs2297723, rs117519669, rs7157977, rs10143899, rs3759779, rs143477571, rs2303221, rs34693726, rs3803752, rs142921938, rs3746295, rs2547319, rs2547318, and rs4911402;
the second SNP site combination related to spherical equivalent of the right eye comprises: one or more of rs4648600, rs1061624, rs2275365, rs35909785, rs3103778, rs2457829, rs10889315, rs78587889, rs12568050, rs12564283, rs528123098, rs139842833, rs74953908, rs1077827, rs12492484, rs75144949, rs3732755, rs3135115, rs16837029, rs149213974, rs17001890, rs56382032, rs12651031, rs2736100, rs7724759, rs754615, rs5369, rs150157174, rs130068, rs45583335, rs78342561, rs2529438, rs1049402, rs2072236, rs2527878, rs2240403, rs78155900, rs114097201, rs2680903, rs59612871, rs3818584, rs16909677, rs10983755, rs2248077, rs2811736, rs2297722, rs2297723, rs9987876, rs7904554, rs907609, rs117519669, rs3750996, rs3802799, rs2276296, rs139692587, rs904628, rs11551723, rs144796593, rs78648016, rs7157977, rs10143899, rs3759779, rs7142098, rs143477571, rs3736911, rs11849022, rs77481241, rs3212112, rs9282879, rs55958706, rs3212056, rs34100926, rs114770841, rs3742368, rs118146919, rs2289702, rs2303221, rs34693726, rs3744566, rs9907420, rs375644567, rs185576254, rs3803752, rs17822735, rs17222523, rs34818467, rs12946397, rs3809724, rs181969864, rs7236574, rs142921938, rs3746295, rs151309111, rs533816037, rs2302948, rs182509214, rs2547319, rs2547318, rs2281209, rs4911402, rs138894461, and rs5756223;
the third SNP site combination related to spherical equivalent of the right eye comprises: one or more of rs16824585, rs4648600, rs17033266, rs1061624, rs202140107, rs2291804, rs2275365, rs139241751, rs35909785, rs6676052, rs3103778, rs116893711, rs116861599, rs2457829, rs10889315, rs17855078, rs78587889, rs140141416, rs4656197, rs45445497, rs143355122, rs12568050, rs12564283, rs10482, rs1929861, rs75401237, rs28517482, rs1264208208, rs2631840, rs188480146, rs528123098, rs61732269, rs367931009, rs139842833, rs79908358, rs79358798, rs1434087, rs4894048, rs13421990, rs13419085, rs143196218, rs139713392, rs6734083, rs150887565, rs4663795, rs74953908, rs143001518, rs6437368, rs147838565, rs1077827, rs144623840, rs78233496, rs9876921, rs6622, rs2279909, rs547922447, rs300977, rs12492484, rs75144949, rs3732755, rs3135115, rs16837029, rs74787220, rs527903313, rs149213974, rs17001890, rs79769348, rs10025654, rs76996680, rs2306369, rs79482316, rs56382032, rs12651031, rs2736100, rs35130836, rs3749684, rs7724759, rs754615, rs45625534, rs3805625, rs137898880, rs147518598, rs202007699, rs2270900, rs75164992, rs17133512, rs76720430, rs5369, rs150157174, rs7756481, rs117298661, rs117644703, rs3094672, rs4713420, rs130068, rs67196728, rs185990098, rs9349593, rs143580489, rs3757302, rs3218602, rs9495370, rs45583335, rs78342561, rs117358940, rs9689983, rs2529438, rs1049402, rs2072236, rs2527878, rs2240403, rs781980620, rs56083327, rs2245368, rs2430307, rs3748126, rs36028884, rs78155900, rs114097201, rs13438494, rs117211799, rs115734214, rs148474510, rs200506987, rs8190955, rs8178175, rs2680903, rs117676215, rs115507207, rs369806130, rs78240711, rs59612871, rs117169590, rs3739523, rs145293869, rs139212809, rs3818584, rs16909677, rs141549766, rs147026086, rs188197626, rs13321, rs10983755, rs2301576, rs7038042, rs11849, rs2275161, rs2248077, rs2811736, rs2297722, rs2297723, rs9987876, rs7904554, rs148582275, rs3740168, rs149647444, rs7100474, rs7079648, rs202166096, rs60420182, rs36104328, rs7098255, rs3814163, rs2297785, rs3736924, rs3736923, rs141296329, rs117520659, rs57285449, rs112687793, rs73401681, rs79948463, rs10902170, rs10902171, rs867839, rs907608, rs907609, rs117519669, rs3750996, rs214086, rs3802799, rs76215382, rs147852038, rs2276296, rs143930161, rs183113103, rs139692587, rs10792769, rs7102675, rs116878689, rs35466364, rs142908193, rs7966459, rs7135745, rs34181394, rs1921038, rs201814780, rs11068531, rs904628, rs11551723, rs7983667, rs144796593, rs140526815_rs77878407, rs78646508, rs34523940, rs9521906, rs78648016, rs7157977, rs10143899, rs3759779, rs7142098, rs41309252, rs535020964, rs3742770, rs3742768, rs116881452, rs143477571, rs61992274, rs142973720, rs530110551, rs3742464, rs3736911, rs11849022, rs77481241, rs3212112, rs9282879, rs55958706, rs3212056, rs34100926, rs114770841, rs3742368, rs144857261_rs373032548, rs189734192, rs188393827, rs118146919, rs562829514, rs1805025, rs8192352, rs2289702, rs150921132, rs61869621, rs1802752, rs113355794, rs7198064, rs2291407, rs9941128, rs9941210, rs9941215, rs9931527, rs12708649, rs13306653, rs11643517, rs5723, rs7196736, rs2303221, rs2304483, rs149645127, rs11550470, rs17232091, rs150889000, rs16954357, rs371756524_rs557159990, rs192103504, rs115314145, rs34693726, rs2277651, rs2277652, rs71360269, rs71360270, rs3744566, rs3744568, rs9907420, rs375644567, rs185576254, rs2158460, rs3803752, rs17822735, rs17222523, rs34818467, rs12946397, rs3809724, rs35476409_rs397978887, rs3744214, rs181969864, rs4789773, rs28561791, rs7236574, rs76597875, rs61749945, rs118033234, rs1052696, rs1052692, rs148018996, rs34488963, rs3745379, rs3745385, rs142921938, rs3746295, rs151309111, rs139271842, rs140566569, rs533816037, rs78237760, rs79621739, rs75990051, rs62107869, rs305974, rs1055099, rs41290128, rs76151738, rs16980091, rs2302948, rs182509214, rs2547319, rs2547318, rs16982743, rs2284385, rs2281209, rs4911402, rs80158178, rs7509972, rs150511237, rs2231391, rs2231390, rs762228, rs72564613, rs191574093, rs138894461, rs5999924, rs5750146, rs5756223, rs3747168, rs148345118, rs910796, rs910797, rs910798, rs738712, rs16445, rs3922872, rs56248708, rs9628315, and rs8137790;
the first SNP site combination related to sphere of the right eye comprises: one or more of rs1929861, rs139842833, rs149213974, rs130068, rs2072236, rs2240403, rs59612871, rs139692587, rs143477571, rs2303221, rs34693726, rs3744566, rs3803752, rs17223038, rs34488963, rs142921938, and rs2547319;
the second SNP site combination related to sphere of the right eye comprises: one or more of rs35909785, rs10889315, rs143355122, rs10482, rs1929861, rs191149396, rs528123098, rs139842833, rs12492484, rs75144949, rs3732755, rs3135115, rs16837029, rs149213974, rs79769348, rs55656741, rs7724759, rs754615, rs137898880, rs147518598, rs75164992, rs17133512, rs76720430, rs3094672, rs4713420, rs130068, rs286560, rs2529438, rs2072236, rs2527878, rs2240403, rs56083327, rs2245368, rs2430307, rs3748126, rs36028884, rs117676215, rs115507207, rs2403730, rs59612871, rs3739523, rs2811736, rs2297722, rs2297723, rs202166096, rs2297785, rs3736924, rs3736923, rs117519669, rs57477011, rs139692587, rs10792769, rs142908193, rs140526815, rs78646508, rs34523940, rs9521906, rs78648016, rs143477571, rs3742464, rs3736911, rs11849022, rs77481241, rs3212112, rs9282879, rs55958706, rs3212056, rs34100926, rs114770841, rs3742368, rs189734192, rs9941210, rs9941215, rs12708649, rs13306653, rs11643517, rs5723, rs2303221, rs11550470, rs2115400, rs150889000, rs192103504, rs2024076, rs34693726, rs2277648, rs2286357, rs12950718, rs2277651, rs2277652, rs71360269, rs71360270, rs3744566, rs3744568, rs3803752, rs17822735, rs17222523, rs34818467, rs12946397, rs3809724, rs3744214, rs7236574, rs1064058, rs1064059, rs17223038, rs34488963, rs3745379, rs3745385, rs142921938, rs3746295, rs2302948, rs2547319, rs2547318, rs4911402, and rs191574093;
the third SNP site combination related to sphere of the right eye comprises: one or more of rs35909785, rs10889315, rs143355122, rs10482, rs1929861, rs191149396, rs528123098, rs139842833, rs12492484, rs75144949, rs3732755, rs3135115, rs16837029, rs149213974, rs79769348, rs55656741, rs7724759, rs754615, rs137898880, rs147518598, rs75164992, rs17133512, rs76720430, rs3094672, rs4713420, rs130068, rs286560, rs2529438, rs2072236, rs2527878, rs2240403, rs56083327, rs2245368, rs2430307, rs3748126, rs36028884, rs117676215, rs115507207, rs2403730, rs59612871, rs3739523, rs2811736, rs2297722, rs2297723, rs202166096, rs2297785, rs3736924, rs3736923, rs117519669, rs57477011, rs139692587, rs10792769, rs142908193, rs140526815, rs78646508, rs34523940, rs9521906, rs78648016, rs143477571, rs3742464, rs3736911, rs11849022, rs77481241, rs3212112, rs9282879, rs55958706, rs3212056, rs34100926, rs114770841, rs3742368, rs189734192, rs9941210, rs9941215, rs12708649, rs13306653, rs11643517, rs5723, rs2303221, rs11550470, rs2115400, rs150889000, rs192103504, rs2024076, rs34693726, rs2277648, rs2286357, rs12950718, rs2277651, rs2277652, rs71360269, rs71360270, rs3744566, rs3744568, rs3803752, rs17822735, rs17222523, rs34818467, rs12946397, rs3809724, rs3744214, rs7236574, rs1064058, rs1064059, rs17223038, rs34488963, rs3745379, rs3745385, rs142921938, rs3746295, rs2302948, rs2547319, rs2547318, rs4911402, and rs191574093;
the first SNP site combination related to cylinder of the right eye comprises: one or more of rs42688, rs143580489, rs10484690, and rs189212243;
the second SNP site combination related to cylinder of the right eye comprises: one or more of rs12408385, rs2275365, rs374309842, rs77901289, rs147707514, rs143100530, rs41298993, rs26173, rs26098, rs1353251, rs5867289, rs1494555, rs42688, rs32210, rs143580489, rs1204114, rs10484690, rs118151455, rs201920957, rs189212243, rs117569740, rs2271001, rs185257411, rs698028, rs4923849, rs12904657, rs62037369, rs13337155, rs17223038, rs199667693, and rs148965154;
the third SNP site combination related to cylinder of the right eye comprises: one or more of rs12408385, rs2275365, rs2275360, rs368345074, rs80002732, rs374309842, rs77901289, rs191506876, rs147707514, rs3087649, rs10177030, rs143100530, rs1434087, rs3748962, rs28900693, rs1077827, rs80341144, rs3213930, rs41298993, rs3749584, rs10517195, rs2271924, rs13147012, rs79482316, rs3827497, rs26173, rs26098, rs26107, rs26086, rs1353251, rs5867289, rs1494555, rs42688, rs32210, rs154483, rs7764126, rs13205210, rs143580489, rs1204114, rs10484690, rs118151455, rs200111157, rs113281844, rs201920957, rs79960636, rs112596877, rs117509972, rs189212243, rs74699763, rs2584, rs7023474, rs117569740, rs147367410, rs61748723, rs17849182, rs79735301, rs145978705, rs304494, rs115526095, rs7907476, rs4880361, rs2271001, rs111885243, rs2074233, rs622082, rs546382, rs185257411, rs145509540, rs144650014, rs17103265, rs11570779, rs17793018, rs76846944, rs698028, rs4923849, rs12904657, rs72765429, rs111980103, rs61737565, rs4788099, rs7198606, rs62037369, rs62037371, rs10499, rs13337155, rs61742982, rs9303742, rs17223038, rs199667693, rs148965154, rs706764, rs56408444, rs117753855, rs78472358, rs6037509, rs150212573, rs147911723, and rs470101;
the fourth SNP site combination related to cylinder of the right eye comprises: one or more of rs3795262, rs12409277, rs61735125, rs61746709, rs191051279, rs151112364, rs2291804, rs12408385, rs2275365, rs2275360, rs146696696, rs11542470, rs12386, rs74622002, rs368345074, rs142732858, rs80002732, rs374309842, rs144649977, rs78780269, rs2275863, rs77901289, rs191506876, rs117369152, rs147707514, rs6715296, rs3087649, rs7584256, rs10177030, rs11694145, rs4149520, rs143100530, rs1434087, rs77497147, rs2004888, rs11568351, rs141302000, rs3748962, rs11574750, rs16858808, rs16858811, rs141559295, rs3769838, rs6734083, rs28900693, rs4663795, rs34401965, rs76999397, rs11919775, rs1869852, rs1869851, rs1077827, rs80341144, rs3213930, rs2029618, rs75318799, rs11719458, rs33988592, rs79845192, rs41298993, rs2124541, rs66748594, rs28372853, rs572169, rs2246901, rs73189480, rs3733217, rs34611001, rs61746000, rs117486807, rs147434354, rs35762546, rs3749584, rs2271036, rs2271037, rs10517195, rs41498446, rs3733324, rs2271924, rs1801612, rs2280099, rs8582, rs13147012, rs79482316, rs3827497, rs26173, rs26098, rs26107, rs26086, rs1051489, rs2270915, rs3813474, rs1353252, rs1353251, rs1494561, rs1353250, rs5867289, rs1494558, rs1494555, rs987106, rs2233216, rs2233213, rs3217552, rs16874505, rs3749684, rs3729668, rs28388868, rs144533176, rs42688, rs32210, rs13180243, rs154483, rs367805, rs2631368, rs117231680, rs10053292, rs7701410, rs11249552, rs142883877, rs150856424, rs3734391, rs9370867, rs77229316, rs17194565, rs117487649, rs1264319, rs35756329, rs17201248, rs190135313, rs7764126, rs11755393, rs13205210, rs3734264, rs1051115, rs74609186, rs6928630, rs143580489, rs1204114, rs10484690, rs118151455, rs143904280, rs9459355, rs9459356, rs113806172, rs10227750, rs200111157, rs539973087, rs182692351, rs13306387, rs79809732, rs113281844, rs201920957, rs236652, rs236655, rs236666, rs79960636, rs112596877, rs117509972, rs772373052, rs801841, rs1062394, rs200333762, rs4301376, rs775410128, rs3987896, rs146160185, rs17171113, rs2788473, rs189212243, rs74699763, rs2584, rs41488144, rs13306550, rs77061383, rs145349678, rs371193886, rs8178175, rs4925828, rs7023474, rs3802392, rs4879781, rs35066989, rs112858544, rs1164863576, rs16909677, rs117738198, rs117569740, rs147367410, rs61748723, rs16916100, rs2297530, rs77309503, rs77564189, rs371047734, rs17849182, rs79735301, rs145978705, rs118012211, rs76191089, rs142547455, rs2073878, rs78774780, rs140120230, rs150198392, rs10600924, rs7921838, rs3829125, rs183963660, rs17134592, rs569927230, rs41382649, rs11255267, rs144121793, rs12773592, rs139646798, rs7095891, rs11003124, rs202046718, rs7076156, rs79298145, rs3758464, rs16935560, rs304494, rs304439, rs116994405, rs11553577, rs61731067, rs7100644, rs115526095, rs7907476, rs2901895, rs7912552, rs11017735, rs4880361, rs3764896, rs1056819, rs729662, rs148238084, rs138312423, rs2271001, rs111885243, rs6485702, rs326222, rs2013867, rs901746, rs2167079, rs7120118, rs1050922, rs2074233, rs77749149, rs78038516, rs11231726, rs12146487, rs182061168, rs514076, rs145270731, rs3741172, rs1551886, rs17856219, rs1551888, rs622082, rs546382, rs488792, rs568862, rs187530720, rs2276278, rs2846412, rs143351376, rs117176605, rs10844777, rs1948516, rs2232566, rs7132546, rs12313730, rs186809868, rs35988455, rs2178685, rs73218270, rs11547907, rs9658501, rs3914, rs555404, rs10773127, rs185257411, rs145509540, rs9315569, rs953958, rs953957, rs2183207, rs4578496, rs4614565, rs151310875, rs377238277, rs144650014, rs2274827, rs17103265, rs11570779, rs12883882, rs17793018, rs76846944, rs2297298, rs698028, rs944050, rs2069590, rs780162072, rs73370219, rs4923849, rs12904657, rs3068, rs1046657, rs12324091, rs3743267, rs12438118, rs556233670, rs149146765, rs193114293, rs2227933, rs3214113, rs72765429, rs34855511, rs400037, rs387467, rs111980103, rs367960426, rs182015467, rs117824484, rs373109045, rs41303763, rs149271, rs180743, rs180744, rs151174, rs181206, rs61737565, rs4788099, rs7198606, rs62037369, rs62037371, rs9931989, rs10499, rs140411458, rs71220135, rs13337155, rs141238615, rs1055708, rs114991387, rs145160385, rs4796407, rs77805790, rs756223, rs61742982, rs117908897, rs9890678, rs11080105, rs3744374, rs56135703, rs117696751, rs2164449, rs150548254, rs7216513, rs148008327, rs9303742, rs148172329, rs148241618, rs12954944, rs8092674, rs146099416, rs8095031, rs2298787, rs17069506, rs17223038, rs968688, rs199667693, rs148965154, rs8102258, rs8112094, rs79579962, rs706764, rs56408444, rs4142943, rs373547599, rs28399491, rs147818341, rs16982743, rs60467951, rs117753855, rs78472358, rs9636153, rs6041749, rs6037509, rs6084431, rs7262903, rs2424943, rs2424944, rs73264111, rs41305787, rs45585744, rs17310782, rs2425007, rs3053708, rs10536288, rs61501849, rs2425014, rs117729108, rs11481073, rs1998233, rs1000411, rs33969356, rs2233884, rs2072974, rs2273086, rs3746619, rs3827103, rs35295928, rs150212573, rs147911723, rs9636833, rs138340460, rs5992854, rs201400555, rs4820658, rs11090413, and rs470101;
the first SNP site combination related to spherical equivalent of the left eye comprises: one or more of rs146160422, rs75054661, rs4959012, rs757262, rs757259, rs145293869, rs34649612, rs7157977, rs563363438, rs2277533, rs7258847, rs78661674, rs5845912, and rs8137790;
the second SNP site combination related to spherical equivalent of the left eye comprises: one or more of rs146160422, rs1051097, rs12129653, rs17553619, rs2275856, rs150943357, rs117712357, rs192688643, rs75054661, rs2292661, rs186309103, rs11556167, rs9790356, rs9790628, rs7717970, rs12519352, rs11133847, rs2304068, rs4959012, rs757262, rs757259, rs929156, rs236444, rs17138865, rs17545405, rs148474510, rs8178175, rs55991828, rs12549018, rs529268660, rs145293869, rs1627784, rs200536689, rs573212, rs4745933, rs3751031, rs12811832, rs11168338, rs2306436, rs3741990, rs34649612, rs7157977, rs17646457, rs563363438, rs2277533, rs2452524, rs4904, rs77707876, rs370627229, rs9929443, rs149149128, rs144944402, rs113276760, rs17682036, rs59176398, rs732109, rs11260004, rs11882197, rs13535, rs2241356, rs57747888, rs397764300_rs58960485, rs16015, rs151309111, rs77561511, rs78422130, rs7258847, rs78661674, rs2183573, rs9981301, rs4624474, rs369859899, rs5845912, rs9628315, rs8138121, and rs8137790;
the third SNP site combination related to spherical equivalent of the left eye comprises: one or more of rs28566954, rs377223831, rs41274480, rs146160422, rs12049046, rs16830693, rs1051097, rs12129487, rs111686948, rs12129653, rs7527057, rs144990398, rs17553619, rs140141416, rs17024525, rs117354680, rs3747626, rs16861256, rs7542316, rs7531786, rs2231241, rs2231240, rs10911390, rs10911391, rs10911392, rs12045762, rs16861394, rs6661715, rs13374714, rs13375853, rs2275856, rs6676149, rs3754234, rs2273584, rs6696123, rs11583387, rs3768066, rs3820553, rs1063128, rs3738521, rs3890478, rs28383065, rs6720415, rs6546511, rs150943357, rs77408734, rs117712357, rs3821036, rs192688643, rs6734083, rs68104327, rs35996697, rs151056400, rs116828761, rs28538922, rs138467334, rs2305613, rs115586700, rs76657333, rs79162067, rs75054661, rs4387990, rs117336796, rs77786095, rs2292661, rs12630803, rs3774765, rs1800766, rs56106326, rs139051035, rs7696197, rs80050773, rs10019009, rs61732380, rs17029087, rs3817083, rs10026790, rs11944752, rs3816873, rs2718684, rs12648093, rs186309103, rs11556167, rs9790356, rs9790628, rs4956981, rs13171103, rs4956987, rs7717970, rs12519352, rs11133847, rs117918201, rs56349002, rs2277042, rs6875303, rs6860528, rs25782, rs117977823, rs11959659, rs2304068, rs192194, rs147518598, rs74403861, rs3734056, rs139635794, rs1611212, rs28400887, rs9261424, rs4959012, rs757262, rs757259, rs929156, rs763046, rs236444, rs73733155, rs2100375, rs1051429, rs17181352, rs8191890, rs61236605, rs142944427, rs3735384, rs17138865, rs17545405, rs140518317, rs2214098, rs148474510, rs555905346, rs6945709, rs10240503, rs2074690, rs374377569, rs200506987, rs6996950, rs145988500, rs6558166, rs149587418, rs8178175, rs55991828, rs117571873, rs77428666, rs12549018, rs529268660, rs1815549, rs139669465, rs145293869, rs1627784, rs139212809, rs200536689, rs77023914, rs573212, rs28369760, rs185006838, rs75361513, rs139337135, rs75911175, rs17148873, rs35535797, rs28434439, rs34630110, rs199902798, rs34296355, rs4745933, rs906220, rs71479686, rs7069048, rs7075840, rs115337638, rs188054898, rs146804890, rs36088476, rs4757987, rs41282940, rs2986407, rs7808, rs3751031, rs201491055, rs145854081, rs202199158, rs117855259, rs117414825, rs75079562, rs1042833, rs17487041, rs12228215, rs12811832, rs11168338, rs1799986, rs2306436, rs17015438, rs45502896, rs34623199, rs140205595, rs11082, rs2305901, rs61941459, rs3741990, rs34649612, rs145055159, rs117478978, rs11551723, rs200011804, rs150666338, rs147350400, rs199627749, rs7157977, rs118004333, rs79020288, rs17646457, rs77215854, rs76611205, rs183718030, rs12915554, rs563363438, rs2277533, rs2452524, rs16963739, rs72738980, rs78309240, rs4904, rs77707876, rs3748380, rs200336355, rs3730278, rs2745136, rs2738893, rs1054645, rs527843401, rs145737959, rs2447927, rs370627229, rs2301770, rs1059676, rs9929443, rs41303763, rs149149128, rs144944402, rs2271047, rs11859725, rs113276760, rs372478569_rs71380707, rs71395334, rs11556634, rs11556635, rs3027234, rs17682036, rs144911043, rs16971620, rs41283411, rs4986763, rs4986764, rs4986765, rs540183826, rs16973424, rs146684394, rs712833, rs73350377, rs6728, rs181969864, rs59176398, rs732108, rs732109, rs76994448, rs75667305, rs28424873, rs28473343, rs3744937, rs368494050, rs146083008, rs11260004, rs11882197, rs2288415, rs10411624, rs13535, rs2217652, rs3826783, rs2230752, rs2241356, rs1978713, rs4804105, rs57747888, rs397764300_rs58960485, rs139817982, rs16015, rs151309111, rs2286663, rs117237657, rs3745764, rs189041504, rs55811458, rs2233156, rs148509022, rs407731, rs406968, rs388706, rs12052105, rs8108435, rs76579307, rs3745403, rs77561511, rs35336528, rs78422130, rs1043673, rs117172918, rs7258847, rs138317120, rs116904482, rs4815467, rs2073194, rs41275624, rs6057110, rs7828, rs1051421, rs6076122, rs78661674, rs6059011, rs148646155, rs5743533, rs114985890, rs80158178, rs201196272, rs8127537, rs2225425, rs2410104, rs1041439, rs2183573, rs1984748, rs9981301, rs4624474, rs73231245, rs885987, rs756661, rs5748471, rs202112368, rs369859899, rs6001008, rs6001009, rs9607524, rs17848337, rs5996087, rs73165153, rs5845912, rs9628315, rs8138121, rs8137790, rs68178377, and rs2232885;
the first SNP site combination related to sphere of the left eye comprises: one or more of rs12549018, rs529268660, rs3741990, rs34649612, rs2452524, rs57747888, rs77561511, rs78422130, rs2073194, rs9981301, rs4624474, rs5845912, and rs8137790;
the second SNP site combination related to sphere of the left eye comprises: one or more of rs377223831, rs146160422, rs12129653, rs16861256, rs7542316, rs2231241, rs2231240, rs10911390, rs10911392, rs12045762, rs16861394, rs6661715, rs150943357, rs192688643, rs76657333, rs79162067, rs4387990, rs1981745, rs17466625, rs139051035, rs61732380, rs17029087, rs3817083, rs10026790, rs2718684, rs186309103, rs7717970, rs12519352, rs11133847, rs2304068, rs3734056, rs139635794, rs4959012, rs757262, rs757259, rs17181352, rs12549018, rs529268660, rs145293869, rs573212, rs117628421, rs139337135, rs522328, rs2228149, rs3751031, rs145854081, rs1042833, rs12228215, rs12811832, rs2305901, rs61941459, rs3741990, rs34649612, rs1060474, rs7157977, rs2277533, rs2452524, rs4904, rs2447927, rs1657074, rs1059676, rs9929443, rs113276760, rs732109, rs11260004, rs11882197, rs10411624, rs13535, rs2217652, rs2241356, rs74351326, rs4804105, rs57747888, rs397764300, rs16015, rs2286663, rs34364867, rs117237657, rs189041504, rs77561511, rs78422130, rs7258847, rs862708, rs4815467, rs2073194, rs6076122, rs78661674, rs2183573, rs9981301, rs4624474, rs9607524, rs5845912, rs9628315, rs8137790, and rs76476219;
the third SNP site combination related to sphere of the left eye comprises: one or more of rs28566954, rs377223831, rs41274480, rs146160422, rs16830693, rs1051097, rs12129487, rs12129653, rs12403630, rs58464768, rs148811681, rs41305862, rs17553619, rs71637410, rs16861256, rs7542316, rs7531786, rs2231241, rs2231240, rs10911390, rs10911391, rs10911392, rs12045762, rs16861394, rs6661715, rs138246957, rs182522436, rs13374714, rs13375853, rs2275856, rs6676149, rs3754234, rs2273584, rs6696123, rs11583387, rs3768066, rs3820553, rs1063128, rs3738521, rs76576389, rs150943357, rs34363861, rs117712357, rs71423514, rs76709099, rs192688643, rs151056400, rs116828761, rs28538922, rs138467334, rs115586700, rs76657333, rs143137431, rs79162067, rs75054661, rs4387990, rs117336796, rs145158569, rs12630803, rs3774765, rs1800766, rs74737650, rs1981745, rs17466625, rs12330397, rs139051035, rs10019009, rs61732380, rs17029087, rs3817083, rs10026790, rs11944752, rs1800591, rs3816873, rs2718684, rs7677415, rs3796936, rs3828506, rs186309103, rs11556167, rs9790356, rs9790628, rs4956981, rs13171103, rs4956987, rs7717970, rs12519352, rs11133847, rs117918201, rs26675, rs1697144, rs2304068, rs147518598, rs57930479, rs140779761, rs74403861, rs3734056, rs139635794, rs10900967, rs1611212, rs28400887, rs9261424, rs4959012, rs757262, rs757259, rs929156, rs3888778, rs75541627, rs117340770, rs9468812, rs60399658, rs189316271, rs186815309, rs11966699, rs73733155, rs3800023, rs2273264, rs6933125, rs17181352, rs8191890, rs142944427, rs1802846, rs1131372, rs3807589, rs200730019, rs17138865, rs17545405, rs775410128, rs555905346, rs6945709, rs2074690, rs200506987, rs3739308, rs3735959, rs145988500, rs6558165, rs6558166, rs149587418, rs55991828, rs151304651, rs77428666, rs141149968, rs12549018, rs529268660, rs7003945, rs200070447, rs1815549, rs139669465, rs145293869, rs1627784, rs573212, rs28369760, rs185006838, rs117628421, rs75361513, rs41415046, rs139337135, rs522328, rs515182, rs482712, rs10760500, rs2228149, rs34630110, rs703075, rs9337951, rs906220, rs906221, rs145472189, rs146884162, rs188054898, rs541533723, rs552775933, rs146804890, rs2280134, rs36088476, rs4757987, rs2986407, rs7808, rs3751031, rs4645933, rs139692587, rs76958954, rs145854081, rs5744247, rs202199158, rs117855259, rs117414825, rs2291075, rs117665966, rs1042833, rs17487041, rs12228215, rs12811832, rs11168338, rs138189784, rs75577568, rs2306436, rs369302829, rs2305901, rs61941459, rs3741990, rs34649612, rs1248046, rs145055159, rs117478978, rs200011804, rs150666338, rs1060474, rs10139379, rs8008670, rs8009789, rs8022395, rs7155123, rs199627749, rs7157977, rs79020288, rs183718030, rs9972231, rs563363438, rs2277533, rs2452524, rs78309240, rs4904, rs77707876, rs1545119, rs3748380, rs200336355, rs2745136, rs60526088, rs2738893, rs1054645, rs2447927, rs1657074, rs370627229, rs2301770, rs1059676, rs9929443, rs55969568, rs71220135, rs16943483, rs11859725, rs113276760, rs5818479, rs71395334, rs78806449, rs1043592, rs17682036, rs144911043, rs16971620, rs540183826, rs16973424, rs146684394, rs80345815, rs732109, rs79712753, rs28424873, rs189184481, rs61735591, rs7251165, rs146083008, rs147472740, rs4134853, rs11260004, rs117622602, rs11882197, rs10411624, rs13535, rs2217652, rs3826783, rs2230752, rs2241356, rs74351326, rs1978713, rs4804105, rs57747888, rs397764300, rs139817982, rs16015, rs151309111, rs2286663, rs34364867, rs117237657, rs189041504, rs1865094, rs2233156, rs2302987, rs148509022, rs188387562, rs407731, rs406968, rs388706, rs12052105, rs3745403, rs34604090, rs77561511, rs35336528, rs78422130, rs7258847, rs862708, rs2361119, rs4801478, rs4815467, rs2073194, rs6076122, rs78661674, rs11546155, rs114985890, rs2236001, rs3989369, rs8127537, rs2225425, rs2410104, rs1041439, rs2183573, rs1984748, rs9981301, rs4624474, rs1051367, rs2073733, rs885985, rs1803545, rs148650930, rs2072012, rs9607524, rs17523588, rs17848337, rs73165153, rs5845912, rs9628315, rs8137790, rs76476219, rs55861809, and rs68178377;
the first SNP site combination related to cylinder of the left eye comprises: one or more of rs26173, rs26098, rs26107, rs26086, rs143580489, rs10484690, and rs117984746;
the second SNP site combination related to cylinder of the left eye comprises: one or more of rs2296474, rs2765015, rs3766165, rs145378993, rs13306137, rs147707514, rs112218972, rs3796381, rs26173, rs26098, rs26107, rs26086, rs987106, rs75126858, rs61734326, rs2278226, rs6928630, rs143580489, rs1204114, rs10484690, rs74699763, rs11574345, rs4925828, rs304494, rs304439, rs304448, rs4763975, rs377238277, rs144650014, rs12438118, rs41303763, rs9797618, rs10420552, rs4802029, rs28512414, rs1975937, rs2075284, rs3095726, rs34785154, rs60704260, rs6084431, rs147911723, rs41279352, rs2427624, and rs117984746;
the third SNP site combination related to cylinder of the left eye comprises: one or more of rs2296474, rs307377, rs2765015, rs3766165, rs145378993, rs61746709, rs114369626, rs4147830, rs540468026, rs80105432, rs45445497, rs13306137, rs191506876, rs147707514, rs4149520, rs4988956, rs4988957, rs10192036, rs10204137, rs4988958, rs10192157, rs10206753, rs3755276, rs112218972, rs3749022, rs76999397, rs35220123, rs80341144, rs3796381, rs117486807, rs147434354, rs76469954, rs26173, rs26098, rs26107, rs26086, rs987106, rs75126858, rs61734326, rs2278226, rs144289000, chr5-140568680, rs7701410, rs117600221, rs150856424, rs3188, rs6928630, rs143580489, rs1204114, rs10484690, rs775410128, rs13225910, rs61733329, rs189212243, rs74699763, rs11574345, rs8178175, rs10092164, rs11539113, rs68008113, rs7462883, rs66881636, rs61747644, rs2721167, rs2620644, rs2250891, rs2250657, rs2721176, rs4925828, rs117569740, rs142643091, rs140966742, rs3758464, rs16935560, rs144762118, rs7899453, rs3740252, rs304494, rs304439, rs304448, rs148238084, rs622082, rs546382, rs189385634, rs4763975, rs2232566, rs7132546, rs5742612, rs35767, rs2861550, rs377238277, rs144650014, rs35405829, rs11569599, rs10130914, rs140879339, rs12438118, rs182015467, rs41303763, rs142805492, rs8055740, rs75743672, rs75277269, rs76896732, rs2164449, rs2230601, rs9303742, rs79577494, rs9797618, rs10420552, rs4802029, rs28512414, rs1975937, rs958305, rs2075284, rs3095726, rs34785154, rs60704260, rs2034891, rs117753855, rs78472358, rs8099961, rs58634535, rs6084431, rs147911723, rs41279352, rs2427624, rs117984746, rs470101, and rs2267163;
the fourth SNP site combination related to cylinder of the left eye comprises: one or more of rs6690013, rs609805, rs621668, rs2274264, rs2274263, rs2296474, rs10907179, rs307349, rs307355, rs35744813, rs307377, rs307371, rs2296471, rs2765015, rs34091023, rs1171, rs3766165, rs145378993, rs115226069, rs61746709, rs2274329, rs2274330, rs3765967, rs148750290, rs114369626, rs35533335, rs74622002, rs145619910, rs141216171, rs146227933, rs145717494, rs4147830, rs3215952, rs2229783, rs17127203, rs1676486, rs1763347, rs368345074, rs7556386, rs540468026, rs374309842, rs942963, rs77181873, rs80105432, rs33941127, rs45445497, rs12026637, rs144649977, rs78780269, rs3813972, rs60555365, rs335524, rs4149230, rs2292559, rs2292561, rs41310561, rs147519768, rs13306137, rs1800672, rs2296801, rs77082151, rs76255469, rs12135078, rs112212885, rs62121538, rs191506876, rs147707514, rs74867758, rs4665385, rs3792253, rs1126497, rs4149520, rs3771175, rs4988956, rs4988957, rs10192036, rs10204137, rs4988958, rs10192157, rs10206753, rs3755276, rs112218972, rs116177854, rs2123465, rs2116664, rs1469651, rs1469650, rs1053394, rs77698806, rs1434087, rs3749022, rs145346406, rs6734083, rs11676792, rs53576, rs76999397, rs2075310, rs35993975, rs33917318, rs17032276, rs17032278, rs17032283, rs35220123, rs9879080, rs7649243, rs34608006, rs6805869, rs9811771, rs2272125, rs2272124, rs7626117, rs7647987, rs6796538, rs35229877, rs11919713, rs1969154, rs1985428, rs3732675, rs80341144, rs181735010, rs3796381, rs76407344, rs11719458, rs2292661, rs79845192, rs74682201, rs77220512, rs35070271, rs79144888, rs76371354, rs78233713, rs117486807, rs147434354, rs6815254, rs3756247, rs1457976435, rs191674075, rs3749584, rs10517195, rs78205702, rs76469954, rs26173, rs26098, rs26107, rs26086, rs987106, rs62387135, rs79420400, rs75126858, rs61734326, rs2278226, rs2228112, rs42688, rs32210, rs6886922, rs118179072, rs144289000, chr5-140568680, rs182889312, rs7701410, rs117600221, rs142883877, rs150856424, rs3734391, rs140750807, rs138075877, rs2274347, rs3188, rs62622418, rs74609186, rs9396251, rs6928630, rs143580489, rs1204114, rs10484690, rs370140172, rs1048886, rs78873069, rs73333875, rs200555224, rs80190153, rs201920957, rs150559408, rs775410128, rs189382707, rs79848686, rs17171113, rs7791608, rs17173702, rs17173703, rs13225910, rs61733329, rs189212243, rs74699763, rs11550509, rs10866818, rs11574345, rs8178175, rs10092164, rs11539113, rs34596274, rs68008113, rs7462883, rs66881636, rs7812496, rs3739287, rs61747644, rs7840439, rs2721167, rs2620644, rs2250891, rs2250657, rs2721176, rs4925828, rs2721190, rs2721189, rs2721172, rs2281747, rs10961672, rs140349388_rs3833713, rs7023474, rs80235406, rs4879781, rs112858544, rs1164863576, rs6560142, rs879369, rs117569740, rs4838184, rs371047734, rs522328, rs515182, rs482712, rs199901947, rs12684476, rs7043416, rs113809617, rs7079747, rs142643091, rs74704054, rs140966742, rs3758464, rs16935560, rs144762118, rs7899453, rs3740252, rs2762528, rs304494, rs304439, rs304448, rs2254619, rs11017735, rs2298192, rs193087745, rs756924, rs2074022, rs739502, rs12577654, rs148238084, rs111885243, rs57477011, rs2074233, rs2067477, rs77749149, rs145270731, rs622082, rs546382, rs189385634, rs2852365, rs1057992, rs642573, rs3018301, rs12272086, rs5103, rs5069, rs377232839, rs201872688, rs4763975, rs150028933, rs2232566, rs7132546, rs2304821, rs148804891, rs5742612, rs35767, rs10778292, rs11112201, rs10847164, rs117739120, rs185257411, rs145509540, rs2861550, rs2282407, rs377238277, rs144650014, rs35405829, rs145005726, rs45585336, rs186889719, rs11569599, rs11570779, rs76846944, rs698028, rs10130914, rs1808975, rs140879339, rs9919918, rs11853872, rs25397, rs3075142_rs397774483, rs12904657, rs12438118, rs7170185, rs17273206, rs3815003, rs2227933, rs74839044, rs34855511, rs577645079, rs76468382, rs117474153, rs367960426, rs182015467, rs41303763, rs187940556, rs13337155, rs138428580, rs1055708, rs62054627, rs1048149, rs62052673, rs142805492, rs8049480, rs758803114, rs8055740, rs138169571, rs7222126, rs11657498, rs200205820, rs4796407, rs2302763, rs138625739, rs201373267, rs2302160, rs34747500, rs56135703, rs28691051, rs200996148, rs75743672, rs729782, rs117696751, rs75277269, rs76896732, rs2164449, rs2230601, rs9303742, rs35114461, rs17223038, rs148965154, rs79577494, rs79579962, rs1054533, rs9797618, rs10420552, rs4802029, rs28512414, rs1975937, rs958305, rs2075284, rs3095726, rs34785154, rs60704260, rs112805435, rs188724242, rs2034891, rs117753855, rs112910446, rs8100225, rs1657535, rs78472358, rs8099961, rs142900000, rs8958, rs58634535, rs6084431, rs2280091, rs2280092, rs3746642, rs235768, rs235769, rs221984, rs2424943, rs2424944, rs73264111, rs41305787, rs45585744, rs2295096, rs2071971, rs117358971, rs112959405, rs1124178, rs3746619, rs3827103, rs35295928_rs886056870, rs150212573, rs147911723, rs41279352, rs2427624, rs139729932, rs117984746, rs2276254, rs141212516, rs7283989, rs11701124, rs8131313, rs35796750, rs143527452, rs45604134, rs1052756, rs1052773, rs2240111, rs165815, rs2073747, rs5762430, rs470101, rs2267163, rs5997714, rs117291362, and rs11553143.

2. The method according to claim 1, wherein, the method for obtaining genotypes of an SNP site combination in step 1 comprises a TaqMan probe method, a sequencing method, a chip method, time of flight mass spectrometer detection, a restriction fragment-length polymorphism method, a single strand conformation polymorphism method, allele-specific PCR, a SNaPshot method, a SNPlex method, denaturing high performance liquid chromatography, and denaturing gradient gel electrophoresis.

3. The method according to claim 1, wherein, the subject is a human of 6-18 years old.

4. The method according to claim 1, wherein, the subject is a human of 6-18 years old in Asia.

5. The method according to claim 1, wherein, the sample is an oral swab, oral mucosal cells, oral exfoliated cells, or saliva.

6. The method according to claim 1, wherein, the corrective lenses comprise orthokeratology lenses or blur-inducing lenses.

7. The method according to claim 1, wherein, the corrective lenses comprise spectacles and contact lenses.

8. The method according to claim 1, wherein, the myopia model is constructed by logistic regression.

9. The method according to claim 8, wherein, the myopia model is determined by the following steps: traversing all values of a threshold, separately establishing models and calculating AUCs, and taking a model with a maximum AUC as a final myopia model.

10. A method for detecting a vision condition or predicting the possibility of developing a vision problem, comprising a step of determining a vision condition of a subject according to detection results for the SNP site combinations according to claim 1.

11. The method according to claim 10, wherein the method comprises the following steps:
a) collecting a sample from a subject;
b) performing SNP detection on the sample; and
c) determining a vision condition of the subject or predicting the possibility of developing a vision problem according to a result for the detection of b).

12. The method according to claim 11, wherein, the sample includes a sample taken directly from the subject, or, a sample subjected to treatment.

13. The method according to claim 12, wherein, the treatment includes purification, extraction of DNA, and amplification of SNP sites.

14. A predictive analysis apparatus for myopia, the apparatus comprising: a memory and a processor, wherein,
the memory is configured to store program instructions;
the processor is configured to invoke the program instructions, which, when executed, perform the method for detecting a vision condition or predicting the possibility of developing a vision problem according to claim 10.
